(19) United States
(12) Patent Application Publication
(10) Pub. No.: US 2024/0130312 A1
(43) Pub. Date: Apr. 25, 2024

(54) Bacteria Conferring Bioprotection and/or Biofertilizer Properties

(71) Applicant: Agriculture Victoria Services PTY LTD, Bundoora, Victoria (AU)

(72) Inventors: Tongda Li, Southbank (AU); Jatinder Kaur, Taylors Hill (AU); Holly Hone, Parkville (AU); Rens Van Essen, Fitzroy North (AU); Ross Mann, Coburg (AU); Timothy Ivor Sawbridge, Coburg (AU); German Carlos Spangenberg, Bundoora (AU)

(73) Assignee: Agriculture Victoria Services PTY LTD, Bundoora, Victoria (AU)

(21) Appl. No.: 18/278,529

(22) PCT Filed: Feb. 23, 2022
(86) PCT No.: PCT/AU2022/050140

§ 371 Date: Aug. 22, 2023

(30) Foreign Application Priority Data
Feb. 25, 2021 (AU) 2021900518

(51) Int. Cl. A01H 3/00; A01H 5/10; A01H 6/46; A01H 17/00; A01N 63/25; A01P 1/00; A01P 3/00; C05F 11/08; C12N 1/20

(52) U.S. Cl. CPC A01H 3/00; A01H 5/10; A01H 6/4624; A01H 6/463; A01H 17/00; A01N 63/25; A01P 1/00; A01P 3/00; C05F 11/08; C12N 1/205; C12R 2001/01

(57) ABSTRACT

The present invention relates to bacteria conferring bioprotection and/or biofertilizer properties to plants into which they are inoculated. More particularly, the present invention relates to endophyte strains of Paenibacillus sp., plants infected with such strains and related methods.

### FIELD OF THE INVENTION

The present invention relates to bacteria conferring bioprotection and/or biofertilizer properties to plants into which they are inoculated. More particularly, the present invention relates to endophyte strains of Paenibacillus sp., plants infected with such strains and related methods.

### BACKGROUND OF THE INVENTION

A relatively unexplored group of microbes known as endophytes, which reside e.g. in the tissues of living plants, offer a particularly diverse source of novel compounds and genes that may provide important benefits to society, and in particular, agriculture.

Endophytes may be fungal or bacterial. Endophytes often form mutualistic relationships with their hosts, with the endophyte conferring increased fitness to the host, often through the production of defence compounds. At the same time, the host plant offers the benefits of a protected environment and nutriment to the endophyte.

Plants and bacteria can establish mutualistic beneficial interactions leading to enhanced performance of agriculturally important crops and pastures. These bacteria are referred to as plant growth-promoting (PGP) bacteria and possess genes conferring beneficial traits to their host plants, such as biofertilisation and/or bioprotection, leading to improved growth and development, stress tolerance (biotic and abiotic) and yield in agricultural plant species. These PGP bacteria have the potential to reduce the use of synthetic pesticides and fertilisers, many of which have adverse impacts on the environment and human health.

Commercial PGP bacterial products based on Pseudomonas spp. and Rhizobium spp. have been available for many years globally. However, there exists a need to identify other microbes that may benefit agriculture.

Plants of the poaceae family are commonly found in association with fungal and bacterial endophytes. However, there remains a general lack of information and knowledge of the endophytes of grasses as well as of methods for the identification and characterisation of novel endophytes and their deployment in plant improvement programs.

Knowledge of such endophytes may allow certain beneficial traits to be exploited in enhanced pastures, or lead to other agricultural advances, e.g. to the benefit of sustainable agriculture and the environment.

Paenibacillus spp. are Gram-positive, facultative anaerobic bacteria that are commonly found in soil from diverse geographic environments. P. polymyxa is a PGP bacterium which is capable of fixing nitrogen, and is used in agriculture. However, there exists a need to identify other Paenibacillus sp. that may benefit agriculture.

There exists a need to overcome, or at least alleviate, one or more of the difficulties or deficiencies associated with the prior art.

### SUMMARY OF THE INVENTION

In one aspect, the present invention provides a substantially purified or isolated endophyte strain isolated from a plant of the Poaceae family, wherein said endophyte is a strain of Paenibacillus sp. which provides bioprotection and/or biofertilizer phenotypes to plants into which it is inoculated.

In a preferred embodiment, the bioprotection and/or biofertilizer phenotype includes production of the bioprotectant compound in the plant into which the endophyte is inoculated. In a preferred embodiment, the bioprotection and/or biofertilizer phenotype is selected from the group consisting of nitrogen fixation, phosphate solubilisation and/or assimilation, production of organic acids and production of secondary metabolites; in the plant into which the endophyte is inoculated.

In a preferred embodiment, the endophyte strain may be strain S02 as described herein and as deposited with The National Measurement Institute of 1/153 Bertie St, Port Melbourne, Victoria 3207 Australia on 19 Feb. 2021 with accession number V21/003092.

In a preferred embodiment, the endophyte strain may be strain S25 as described herein and as deposited with The National Measurement Institute of 1/153 Bertie St, Port Melbourne, Victoria 3207 Australia on 19 Feb. 2021 with accession number V21/003093.

As used herein the term “endophyte” is meant a bacterial or fungal strain that is closely associated with a plant. By “associated with” in this context is meant that the bacteria or fungus lives on, in or in close proximity to a plant. For example, it may be endophytic, for example living within the internal tissues of a plant, or epiphytic, for example growing externally on a plant.

As used herein the term “substantially purified” is meant that an endophyte is free of other organisms. The term includes, for example, an endophyte in axenic culture. Preferably, the endophyte is at least approximately 90% pure, more preferably at least approximately 95% pure, even more preferably at least approximately 98% pure, even more preferably at least approximately 99% pure.

As used herein the term ‘isolated’ means that an endophyte is removed from its original environment (e.g. the natural environment if it is naturally occurring). For example, a naturally occurring endophyte present in a living plant is not isolated, but the same endophyte separated from some or all of the coexisting materials in the natural system, is isolated.

As used herein the term “bioprotection and/or biofertilizer” means that the endophyte possesses genetic and/or metabolic characteristics that result in a beneficial phenotype in a plant harbouring, or otherwise associated with, the endophyte. Such beneficial properties include improved resistance to pests and/or diseases, improved tolerance to water and/or nutrient stress, enhanced biotic stress tolerance, enhanced drought tolerance, enhanced water use efficiency, reduced toxicity and enhanced vigour in the plant with which the endophyte is associated, relative to an organism not harbouring the endophyte or harbouring a control endophyte such as standard toxic (ST) endophyte.

The pests and/or diseases may include, but not limited to, bacterial and/or fungal pathogens, preferably fungal. In a particularly preferred embodiment, the endophyte may result in the production of the bioprotectant compound in the plant with which it is associated.

As used herein, the term ‘bioprotectant compound’ is meant as a compound that provides bioprotection to the plant or aids the defence of the plant with which it is associated against pests and/or diseases, such as bacterial and/or fungal pathogens. A bioprotectant compound may also be known as a ‘biocidal compound’.

As used herein, the term “organic acids” is meant any bioprotectant compound containing an acid functional group, wherein said compound is produced by a plant. An “organic acid” may include Indole-3-acetic acid (IAA) or any other phytohormone compound produced by a plant.

In a preferred embodiment, the bioprotection and/or biofertilizer phenotype is a result of the differential gene expression of one or more gene(s) selected from nitrogen fixation genes as shown in FIGS. 5a to 5ac (SEQ ID NOS: 3 to 31, respectively) or FIGS. 6a to 6ac (SEQ ID NOS: 32 to 60, respectively).

In a preferred embodiment, the bioprotection and/or biofertilizer phenotype is a result of the differential gene expression of one or more of the gene(s) selected from phosphate solubilisation, phosphonate cluster (phn) and/or phosphate transporter (pst) genes as shown in FIGS. 5a to 5ac (SEQ ID NOS: 3 to 31, respectively) or FIGS. 6a to 6ac (SEQ ID NOS: 32 to 60, respectively).

In a preferred embodiment, the bioprotection and/or biofertilizer phenotype is a result of the differential gene expression of one or more gene(s) selected from indole-3-acetic acid (IAA) production genes as shown in FIGS. 5a to 5ac (SEQ ID NOS: 3 to 31, respectively) or FIGS. 6a to 6ac (SEQ ID NOS: 32 to 60, respectively).

In a preferred embodiment, the bioprotection and/or biofertilizer phenotype includes resistance to harmful fungal pathogen growth.

In a preferred embodiment, the harmful fungal pathogen is selected from Fusarium spp., Verticillium albo-atrum, Monilia persoon, Alternaria mali, Botrytis cinerea, and Aspergillus niger.

In a preferred embodiment, the harmful fungal pathogen is selected from Colletotrichum graminicola and Fusarium verticillioides.

In a preferred embodiment, the bioprotection and/or biofertilizer phenotype includes production of secondary metabolites with antimicrobial bioactivity.

In a preferred embodiment, the secondary metabolite is a result of differential gene expression of one or more gene cluster(s) as shown in FIGS. 9a to 9f (SEQ ID NOS: 61 to 66, respectively).

In a particularly preferred embodiment, the endophyte produces a bioprotectant compound and provides bioprotection to the plant against bacterial and/or fungal pathogens. The terms bioprotectant, bioprotective and bioprotection (or any other variations) may be used interchangeably herein.

Thus, in a preferred embodiment, the present invention provides a method of providing bioprotection to a plant against bacterial and/or fungal pathogens, said method including infecting the plant with an endophyte as hereinbefore described and cultivating the plant.

The endophyte according to the invention may be suitable as a biofertilizer to improve the availability of nutrients to the plant with which the endophyte is associated, including but not limited improved tolerance to nutrient stress.

Thus, in a preferred embodiment, the present invention provides a method of providing biofertilizer to a plant, said method including infecting the plant with an endophyte as hereinbefore described and cultivating the plant.

The nutrient stress may be lack of or low amounts of a nutrient such as phosphate and/or nitrogen. The endophyte may be capable of growing in conditions such as low nitrogen and/or low phosphate and enable these nutrients to be available to the plant with which the endophyte is associated.

The endophyte may result in the production of organic acids and/or the solubilisation of phosphate in the plant with which it is associated and/or provide a source of phosphate to the plant.

Alternatively, or in addition, the endophyte may be capable of nitrogen fixation. Thus, if endophyte is capable of nitrogen fixation, the plant in which the endophyte is associated is capable of growing in low nitrogen conditions and/or the endophyte provides a source of nitrogen to the plant.

As used herein the term “plant of the Poaceae family” is a grass species, particularly a pasture grass such as ryegrass (Lolium) or fescue (Festuca), more particularly perennial ryegrass (Lolium perenne L.) or tall fescue (Festuca arundinaceum, otherwise known as Lolium arundinaceum).

In another aspect, the present invention provides a plant or part thereof infected with an endophyte as hereinbefore described. In preferred embodiments, the plant or part thereof infected with the endophyte may produce bioprotectant compound as hereinbefore described.

Also in preferred embodiments, the plant or part thereof includes an endophyte-free host plant or part thereof stably infected with said endophyte.

The plant inoculated with the endophyte may be a grass or non-grass plant suitable for agriculture, specifically a forage, turf, bioenergy grass, or a grain crop or industrial crop.

The forage, turf or bioenergy grass may be those belonging to the Brachiaria-Urochloa species complex (panic grasses), including Brachiaria brizantha, Brachiaria decumbens, Brachiaria humidicola, Brachiaria stolonifera, Brachiaria ruziziensis, B. dictyoneura, Urochloa brizantha, Urochloa decumbens, Urochloa humidicola, Urochloa mosambicensis as well as interspecific and intraspecific hybrids of Brachiaria-Urochloa species complex such as interspecific hybrids between Brachiaria ruziziensis x Brachiaria brizantha, Brachiaria ruziziensis x Brachiaria decumbens, [Brachiaria ruziziensis x Brachiaria decumbens] x Brachiaria brizantha, [Brachiaria ruziziensis x Brachiaria brizantha] x Brachiaria decumbens.

The forage, turf or bioenergy grass may also be those belonging to the genera Lolium and Festuca, including L. perenne (perennial ryegrass) and L. arundinaceum (tall fescue) and L. multiflorum (Italian ryegrass).

The grain crop or industrial crop species may be selected from the group consisting of, for example, wheat, barley, oats, chickpeas, triticale, fava beans, lupins, field peas, canola, cereal rye, vetch, lentils, millet/panicum, safflower, linseed, sorghum, sunflower, maize, canola, mungbeans, soybeans and cotton.

The grain crop or industrial crop may be a grass belonging to the genus Triticum, including T. aestivum (wheat), those belonging to the genus Hordeum, including H. vulgare (barley), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), and those belonging to the genera Miscanthus, Paspalum, Pennisetum, Poa, Eragrostis and Agrostis.

Alternatively, or in addition, the grain crop may be a non-grass species, for example, any of soybeans, cotton and grain legumes, such as lentils, field peas, fava beans, lupins and chickpeas, as well as oilseed crops, such as canola.

A plant or part thereof may be infected by a method selected from the group consisting of inoculation, breeding, crossing, hybridisation, transduction, transfection, transformation and/or gene targeting and combinations thereof.

Without wishing to be bound by theory, it is believed that the endophyte of the present invention may be transferred through seed from one plant generation to the next. The endophyte may then spread or locate to other tissues as the plant grows, i.e. to roots. Alternatively, or in addition, the endophyte may be recruited to the plant root, e.g. from soil, and spread or locate to other tissues.

Thus, in a further aspect, the present invention provides a plant, plant seed or other plant part derived from a plant or part thereof as hereinbefore described. In preferred embodiments, the plant, plant seed or other plant part may produce a bioprotectant compound, as hereinbefore described.

In another aspect, the present invention provides the use of an endophyte as hereinbefore described to produce a plant or part thereof stably infected with said endophyte. The present invention also provides the use of an endophyte as hereinbefore described to produce a plant or part thereof as hereinbefore described.

In another aspect, the present invention provides a bioprotectant compound produced by an endophyte as hereinbefore described, or a derivative, isomer and/or a salt thereof.

The bioprotectant compound may be produced by the endophyte when associated with a plant, e.g. a plant of the Poaceae family as described above.

Thus, in another aspect, the present invention provides a method for producing a bioprotectant compound, or a derivative, isomer and/or a salt thereof, said method including infecting a plant with an endophyte as hereinbefore described and cultivating the plant under conditions suitable to produce the bioprotectant compound. The endophyte-infected plant or part thereof may be cultivated by known techniques. The person skilled in the art may readily determine appropriate conditions depending on the plant or part thereof to be cultivated.

The bioprotectant compound may also be produced by the endophyte when it is not associated with a plant. Thus, in yet another aspect, the present invention provides a method for producing a bioprotectant compound, or a derivative, isomer and/or a salt thereof, said method including culturing an endophyte as hereinbefore described, under conditions suitable to produce the bioprotectant compound.

The conditions suitable to produce the bioprotectant compound may include a culture medium including a source of carbohydrates. The source of carbohydrates may be a starch/sugar-based agar or broth such as potato dextrose agar, potato dextrose broth or half potato dextrose agar or a cereal-based agar or broth such as oatmeal agar or oatmeal broth. Other sources of carbohydrates may include endophyte agar, Murashige and Skoog with 20% sucrose, half V8 juice/half PDA, water agar and yeast malt extract agar. The endophyte may be cultured under aerobic or anaerobic conditions and may be cultured in a bioreactor.

In a preferred embodiment of this aspect of the invention, the method may include the further step of isolating the bioprotectant compound or a derivative, isomer and/or a salt thereof from the plant or culture medium.

The endophyte of the present invention may display the ability to solubilise phosphate. Thus, in yet another aspect, the present invention provides a method of increasing phosphate use efficiency or increasing phosphate solubilisation by a plant, said method including infecting a plant with an endophyte as hereinbefore described, and cultivating the plant.

In yet another aspect, the present invention provides a method of reducing phosphate levels in soil, said method including infecting a plant with an endophyte as hereinbefore described, and cultivating the plant in the soil.

The endophyte of the present invention may be capable of nitrogen fixation.

Thus, in yet another aspect, the present invention provides a method of growing the plant in low nitrogen containing medium, said method including infecting a plant with an endophyte as hereinbefore described, and cultivating the plant. Preferably, the low nitrogen medium is low nitrogen containing soil.

In yet a further aspect, the present invention provides a method of increasing nitrogen use efficiency or increasing nitrogen availability to a plant, said method including infecting a plant with an endophyte as hereinbefore described and cultivating the plant.

In yet another aspect, the present invention provides a method of reducing nitrogen levels in soil, said method including infecting a plant with an endophyte as hereinbefore described and cultivating the plant in the soil.

The endophyte-infected plant or part thereof may be cultivated by known techniques. The person skilled in the art may readily determine appropriate conditions depending on the plant or part thereof to be cultivated.

The production of a bioprotectant compound has particular utility in agricultural plant species, in particular, forage, turf or bioenergy grasses, or grain crop or industrial crop species. These plants may be cultivated across large areas of e.g. soil where the properties and biological processes of the endophyte as hereinbefore described and/or bioprotectant compound may be exploited at scale.

The part thereof of the plant may be, for example, a seed.

In preferred embodiments, the plant is cultivated in the presence of soil phosphate and/or nitrogen, or alternatively or in addition to applied phosphate and/or applied nitrogen. The applied phosphate and/or applied nitrogen may be by way of, for example, fertiliser. Thus, preferably, the plant is cultivated in soil.

In preferred embodiments, the endophyte may be a Paenibacillus sp. strain S02 as described herein and as deposited with The National Measurement Institute of 1/153 Bertie St, Port Melbourne, Victoria 3207 Australia on 19 Feb. 2021 with accession number V21/003092.

In preferred embodiments, the endophyte may be a Paenibacillus sp. strain S25 as described herein and as deposited with The National Measurement Institute of 1/153 Bertie St, Port Melbourne, Victoria 3207 Australia on 19 Feb. 2021 with accession number V21/003093.

Preferably, the plant is a forage grass, turf grass, bioenergy grass, grain crop or industrial crop, as hereinbefore described.

The part thereof of the plant may be, for example, a seed.

In preferred embodiments, the plant is cultivated in the presence of soil phosphate and/or applied phosphate. The applied phosphate may be by way of, for example, fertiliser. Thus, preferably, the plant is cultivated in soil.

Alternatively, or in addition, the plant is cultivated in the presence of soil nitrogen and/or applied nitrogen. The applied nitrogen may be by way of, for example, fertiliser. Thus, preferably, the plant is cultivated in soil.

In this specification, the term ‘comprises’ and its variants are not intended to exclude the presence of other integers, components or steps.

In this specification, reference to any prior art in the specification is not and should not be taken as an acknowledgement or any form of suggestion that this prior art forms part of the common general knowledge in Australia or any other jurisdiction or that this prior art could reasonably expected to be combined by a person skilled in the art.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE DRAWINGS/FIGURES

FIG. 1—16S rRNA gene sequence of novel Paenibacillus sp. strain S02 (SEQ ID NO: 1).

FIG. 2—16S rRNA gene sequence of novel Paenibacillus sp. strain S25 (SEQ ID NO: 2).

FIG. 3—Dendrogram and associated heatmap depicting the Average Nucleotide Identity between the genomes of novel strains S02 and S25 and 44 P. polymyxa strains publicly available on NCBI. (C1—Clade 1; C2—Clade 2; C3—Clade 3; S02 and S25 strain labels—novel strains isolated in this study; Light grey strain labels—P. polymyxa strains with complete circular genome sequences; P. polymyxa ATCC 842 strain label—the type strain of P. polymyxa).

FIG. 4—Phylogeny of Paenibacillus polymyxa strains (13) and novel bacterial strains S02 and S25 (asterisks), based on a pan-genome Roary analysis. The maximum-likelihood tree was inferred based on 2059 genes conserved among 15 genomes. Values shown next to branches were the local support values calculated using 1000 resamples with the Shimodaira-Hasegawa test.

FIGS. 5a to 5ac—Plant growth promoting genes of novel bacterial strain S25, including nitrogen fixation, phosphate solubilisation, phosphonate cluster, phosphate transporter, IAA production genes (SEQ ID NOS: 3 to 31, respectively).

FIGS. 6a to 6ac—Plant growth promoting genes of novel bacterial strain S02, including nitrogen fixation, phosphate solubilisation, phosphonate cluster, phosphate transporter, IAA production genes (SEQ ID NOS: 32 to 60, respectively).

FIG. 7. PCA of the transcriptome profiles of novel strains S02 (A) and S25 (B) when grown in media with nitrogen (N) and without nitrogen (N-free). Circles represent clusters of replicates from N and N-free treatments.

FIG. 8—PCA of the transcriptome profiles of novel strains S02 (A) and S25 (B) when grown in media with the plant pathogenic fungus Fusarium verticillioides (42586-NB) and without the pathogen (C-NB). Circles represent clusters of replicates from with and without the pathogen treatments.

FIGS. 9a to 9f—Secondary metabolite gene cluster of novel bacterial strain S02 involved in the biosynthesis of fusaricidin B. Core genes within the cluster are CGFHABJE_00078 Plipastatin synthase subunit C—fusG) and CGFHABJE_00083 (D-alanine—D-alanyl carrier protein ligase—fusA) (SEQ ID NOS: 61 to 66, respectively).

FIG. 10. PCA of the transcriptome profiles of barley seedling roots when co-incubated with novel Paenibacillus sp. strains S02 (diagonal stripes) and S25 (horizontal stripes), E. gerundensis AR (vertical stripes), or in Nutrient Broth (NB) (dots).

FIG. 11. Regulated expression of 4332 conserved genes of the two Paenibacillus sp. strains (S02 and S25) when co-incubated with barley seedlings in NB. Number of genes and the corresponding percentage of total conserved genes were shown for each category.

FIG. 12. A Venn diagram showing the amount of barley genes that were differentially expressed in roots during the plant-bacteria interactions assay for all three strains, grown in NB. A total of 22015 genes were differentially expressed. AR: novel E. gerundensis strain; S02/S25: novel Paenibacillus sp. strains.

### DETAILED DESCRIPTION OF THE EMBODIMENTS

The invention comprises two novel plant associated Paenibacillus sp. bacterial strains S02 and S25 isolated from perennial ryegrass (Lolium perenne) plants that have bioprotection and biofertilizer activity. The genomes of the two novel Paenibacillus sp. bacterial strains have been sequenced and are shown to be a novel species, related to Paenibacillus polymyxa. The biofertilisation activity is supported by genomic evidence including genes associated with nitrogen fixation, phosphate solubilisation and phytohormone production. Furthermore, S02 has elevated expression of the nitrogen fixation gene cluster compared to strain S25, particularly under low nitrogen. The bioprotection activity is supported by antifungal bioactivity exhibited in in vitro bioassays, particularly S02 which controlled Fusarium oxysporum and Colletotrichum graminicola unlike S25. Furthermore, the bioprotection activity was supported by genomic evidence including 16 secondary metabolite genes clusters, of which one has been reported to have antifungal activity (fusaricidin B). Transcriptomic evidence identified 5 highly upregulated secondary metabolite gene clusters in S02 compared to S25, including fusaricidin B. Transcriptomic evidence also supported the mutualistic interaction between the two novel Paenibacillus sp. strains and barley, particularly strain S02 which had a transcriptomic profile similar to uninoculated plants and promoted nitrogen metabolism, whereas strain S25 induced stress related genes.

### Example 1—Isolation of Bacterial Strains

A PCR assay was designed to detect the presence of seed-associated N-fixing bacteria by amplifying the nifH gene, which regulates the production of the nitrogenase enzymes. Approximately 1000 perennial ryegrass seeds (L. perenne, cultivar Alto, with standard endophytes, Barenbrug Agriseeds NZ) were washed using sterile water and then ground and soaked in 30 mL Burk's N-free medium (MgSO₄, 0.2 g/L; K₂HPO₄, 0.8 g/L; KH2PO4, 0.2 g/L; CaSO4, 0.13 g/L; FeCl3, 0.00145 g/L; Na2MoO4, 0.000253 g/L; sucrose, 20 g/L). The suspension was incubated for 2 days at 26° C. and 200 rpm, and then serial diluted using sterile Burk's N-free medium (1:10, 100 μL in 900 μL, 8 replicates per dilution). Genomic DNA was extracted from the 10-2 and 10-3 dilutions using a Wizard® Genomic DNA Purification Kit (A1120, Promega, Madison, WI, USA). PCR conditions were as per Reeve et al (2010). In brief, OneTaq® Hot Start 2× Master Mix (M0484, Promega, Madison, WI, USA) was used with a universal nifH gene PCR primer pair (IGK3: 5′-GCIVVTHTAYGGIAARGGIGGIATHGGIAA-3′; DVV: 5′-ATIGCRAAICCICCRCAIACIACRTC-3′; final concentration=0.4 μM) (Gaby and Buckley, 2012) and 50 ng of template DNA (Nuclease-free water—negative control; Rhizobium leguminosarum bv. trifolii WSM1325—positive control). PCR products (˜400 bp fragment) were visualised on an Agilent 2200 TapeStation (Agilent Technologies, Santa Clara, CA, USA), sequenced by Macrogen and analysed using BLAST (Camacho et al., 2009). Dilutions that produced amplicons were sequenced with MinION long read sequencing using the Oxford Nanopore Technologies (ONT) ligase-based library preparation kit (SQK-LSK109, ONT, Oxford, UK) and sequenced on a MinION Mk1B platform (MIN-101B) with R10 flow cells (FLO-MIN110). Genomic sequence data (raw read signals) were basecalled using ONT's Guppy software (Version 3.4.3, HAC basecalling model), and assessed for quality using NanoPlot (De Coster et al., 2018). Basecalled data was filtered to remove adapter sequences using Porechop (Version 0.2.3, https://github.com/rrwick/Porechop), while reads shorter than 300 bp and the worst 5% of reads (based on quality) were discarded using Filtlong (Version 0.2.0, https://github.com/rrwick/FiltIong). Sequencing reads were compared to genomic references using Kraken2 (Wood et al, 2019). In addition, 50 μL of these samples were inoculated in vials containing 5 mL of Burk's N-free semi-solid medium (supplemented with 1.6 g/L agar), and incubated for up to 5 days at 26° C. Cultures were checked daily for a band of microbial growth below the surface of medium, which indicated the presence of N-fixing bacteria (Baldani et al., 2014). Microbes were streaked onto Burk's N-free solid medium [supplemented with 15 g/L agar and 100 IU/mL polymyxin B (P4932-1MU, Sigma-Aldrich, St. Louis, MO, USA)] and incubated for up to 5 days at 26° C. to isolate pure colonies.

Amplicons of the expected size (˜400 bp) were produced from two of eight replicates of the 10-2 dilution, while no amplicon was produced from all eight replicates of the 10-3 dilution. Amplicons were identified as partial sequences of the nifH gene of Paenibacillus polymyxa CR1 (Accession ID: CP006941.2, 1,087,670 bp to 1,088,026 bp; coverage=97%, identity=99%) using BLASTn search against the nr database. Long read data from these dilutions were classified as Bacillus spp. (high abundance), Pseudomonas spp., Massilia spp. and P. polymyxa (low abundance). Culturing on Burk's N-free solid medium with polymyxin B resulted in the purification of two bacterial strains (S02 and S25). Both strains are rod-shaped and Gram-positive, and form heaped, small- to medium-sized colonies on Burk's N-free solid media. Strain S02 produces white and mucoid colonies, while strain S25 produces translucent colonies. Both strains were stored in 15% glycerol at −80° C.

### Example 2—Identification of Novel Paenibacillus sp. Bacterial Strains

Genomic DNA was extracted from overnight cultures using a Wizard® Genomic DNA Purification Kit (A1120, Promega, Madison, WI, USA). Genomic sequencing libraries (Illumina short reads) were prepared from the DNA using the PerkinElmer NEXTFLEX® Rapid XP DNA-Seq Kit (Cat #NOVA-5149-03) and sequenced on an Illumina NovaSeq 6000 platform. Genomic sequence data (raw reads) were assessed for quality and filtered to remove any adapter and index sequence, and low quality bases using fastp (Chen et al., 2018) with the following parameters: -w 8-3 -5. In addition, genomic libraries, sequencing and quality control (MinION long reads) were prepared as per Example 1.

The whole genome of bacterial strains were assembled with filtered long and short reads using Unicycler (Wick et al., 2017). Long reads were used for primary assembly and to resolve repeat regions in the genome, whereas short reads were used to correct small base-level errors. Assembly graphs were visualised using Bandage (Wick et al., 2015). Assembled genomes were taxonomically classified by Kraken2 (Wood and Salzberg, 2014) using a custom database containing all completed bacterial reference genomes in NCBI (20/03/2020). The assembled genome of bacterial strains were annotated using Prokka (Seemann, 2014) with a custom Paenibacillus protein database (based on Kraken2 classification) to predict genes and corresponding functions. A total of 2,536,823,196 bp short reads and 13,203,686,400 bp long reads were generated for both novel strain S02 and S25. Complete circular genome sequences were produced for both novel strains. The genome size of novel strain S02 was 6,060,529 bp (5310 CDSs) with a G+C content of 45.60%, while the genome size of novel strain S25 was 5,958,851 bp (5177 CDSs) with a G+C content of 45.72% (Table 1). There were no plasmids present in either strain

TABLE 1
General genomic characteristics of novel strains S02 and S25

| | Genome | GC | | | | | |
|---|---|---|---|---|---|---|---|
| | size | content | No. of | No. of | No. of | No. of | No. of |
| Strain ID | (bp) | (%) | tRNA | tmRNA | rRNA | gene | CDS |
| S02 | 6,060,529 | 45.60 | 92 | 1 | 33 | 5436 | 5310 |
| S25 | 5,958,851 | 45.72 | 92 | 1 | 36 | 5306 | 5177 |

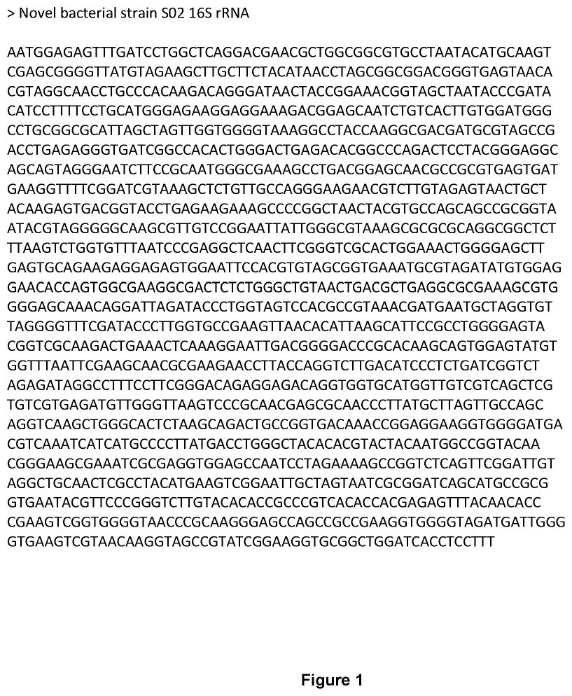

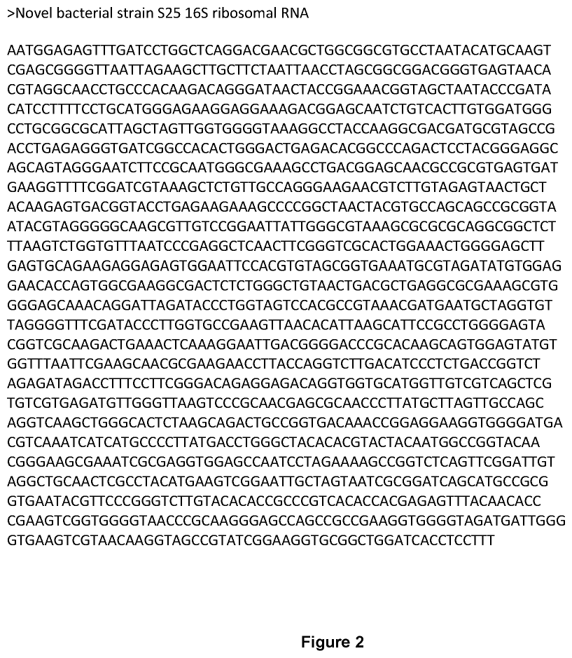

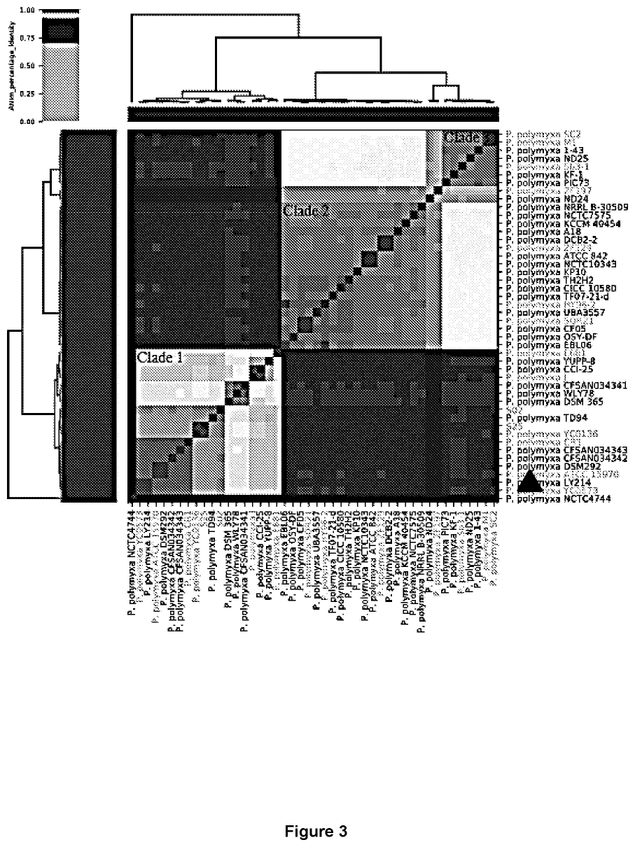

The 16S rRNA gene sequences for novel strains S02 and S25 were identified in the genomes and used for preliminary phylogenetic placement (FIGS. 1 and 2; SEQ ID NOS: 1 and 2, respectively). The sequences showed both novel strains were phylogenetically related to P. polymyxa DSM36 (Genbank Accession: NR_117732.2) with a sequence homology of 99.45% and coverage of 100%. The close relationship between the two strains and P. polymyxa was confirmed by genome-based identifications where both strains were classified by Kraken2 as P. polymyxa E681 (NCBI:txid 349520). Further genomic analysis compared the average nucleotide identity (ANI) of novel strains S02 and S25 to 44 P. polymyxa genomes publicly available on NCBI using the python package pyani (Version 0.2.8, https://widdowquinn.github.io/pyani/). The ANI dendrogram-heatmap revealed three major clades, with Clade 1 comprising 18 strains including novel strains S02 and S25, Clade 2 comprising 18 strains including the type strain (ATCC 842), Clade 3 comprising 7 strains, and strain NCTC4744 forming an outgroup (FIG. 2). Strains in Clade 1 shared between 95-99% ANI, while strains in Clade 2 had >98% ANI, and strains in Clade 3 had >98% ANI. Clades 2 and 3 had <95.5% ANI with one another, while Clade 1 was further separated from the other two Clades sharing <91% ANI. As such, Clade 1 (including novel strains S02 and S25) represents a new Paenibacillus species, given that the ANI is lower than the ANI species boundary which is 95-96% (Richter and Rossello-Mora, 2009; Chun et al., 2018). Similarly, Clade 2 could also represent a new Paenibacillus species, based on this ANI species boundary. The novel Paenibacillus sp. strains S02 and S25 had an ANI of 97.78% to one another, with strain S02 most similar to strain TD94 (98.11% ANI) that was isolated from Scutellaria spp. rhizophore (Xie et al., 2014), and strain S25 most similar to strain YC0136 (99.29% ANI) that was isolated from the tobacco rhizosphere (Liu et al., 2017).

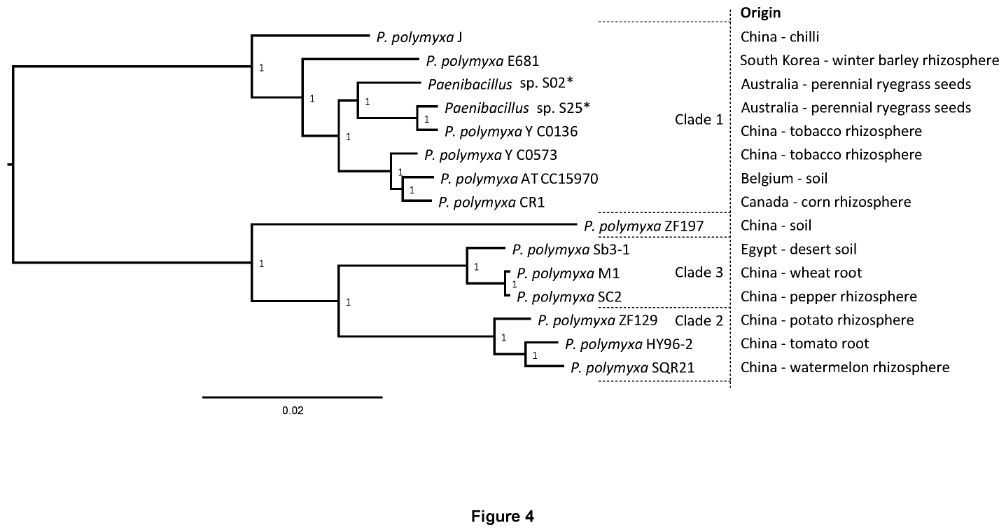

A pan-genome analysis was conducted comparing novel strains S02 and S25 to 13 P. polymyxa strains with complete circular genome sequences. All strains were annotated de novo using the method described above, and compared using Roary to identify shared genes (>95% protein sequence similarity) (Page et al., 2015). A maximum-likelihood phylogenetic tree was inferred using FastTree (Price et al., 2010) with Jukes-Cantor Joins distances, the Generalized Time-Reversible substitution model and the CAT approximation model. Local branch support values were calculated using 1000 resamples with the Shimodaira-Hasegawa test. The pan genome Roary analysis identified 2059 shared genes by all 15 strains. A maximum-likelihood phylogenetic tree was inferred based on the sequence homology of the shared genes. The topology of the phylogenetic tree consisted on three major clades, and was consistent with the ANI analysis (FIG. 4). All clades were separated with 100% local support. Clade 1 consisted of 8 strains, including the two novel strains S02 and S25, and was distinctly separated from Clades 2 and 3 at the root node. Clade 2 and 3 formed adjoining clades on the same primary root node, and each had 3 strains. Strain ZF197 also clustered with Glade 2 and 3 but formed its own branch. Clade 1 consisted of strains from across a broad geographic range, including Asia (China, South Korea), the Pacific (Australia), North America (Canada) and Europe (Belgium), whereas Clades 2 and 3 were largely from Asia (China), except strain Sb3-1 that was from Egypt. All strains were either associated with plants or soil.

### Example 3—Genome Sequence Features Supporting the Biofertilizer Niche of the Novel Bacterial Strains S02 and S25

The presence of plant growth-promoting (PGP) genes in the annotated genomes of S02 and S25 was assessed. PGP genes previously reported in P. polymyxa strains (Eastman et al., 2014; Xie et al., 2016) were targeted (30 genes), including biological nitrogen fixation (9 genes), phosphate solubilisation and assimilation (17 genes) and indole-3-acetic acid production and transportation (4 genes). The PGP gene identification compared the sequence homology of genes from S02 and S25 with closely related P. polymyxa strain CR1 using BLAST (Camacho et al., 2009) (blastn and tblastn, e value >1e⁻¹⁰).

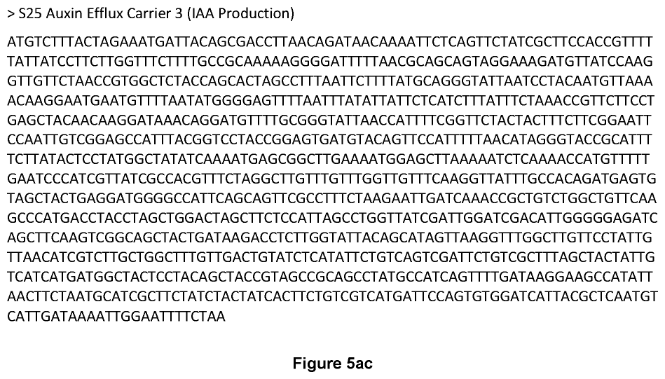

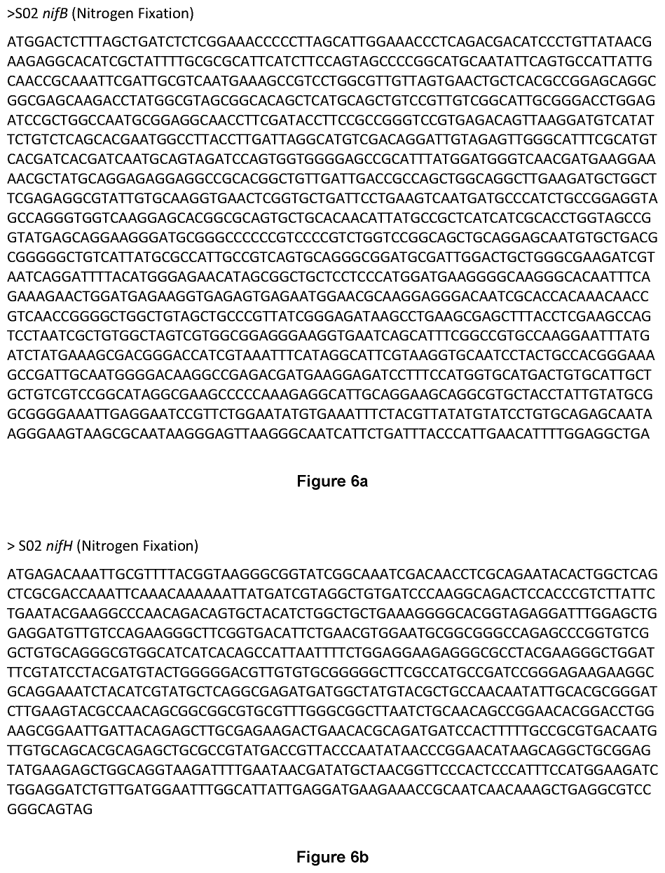

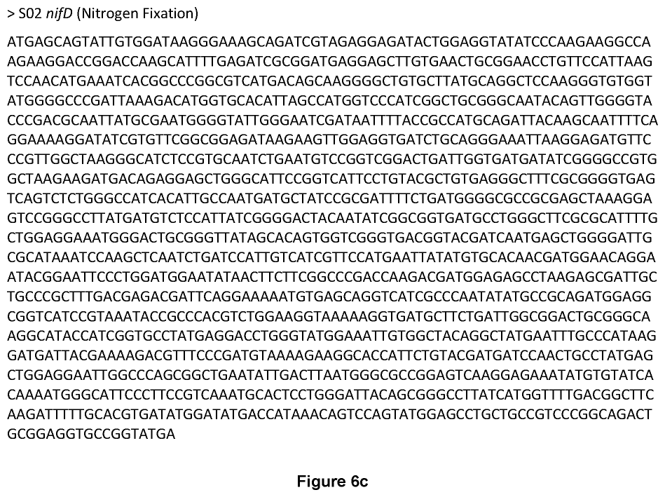

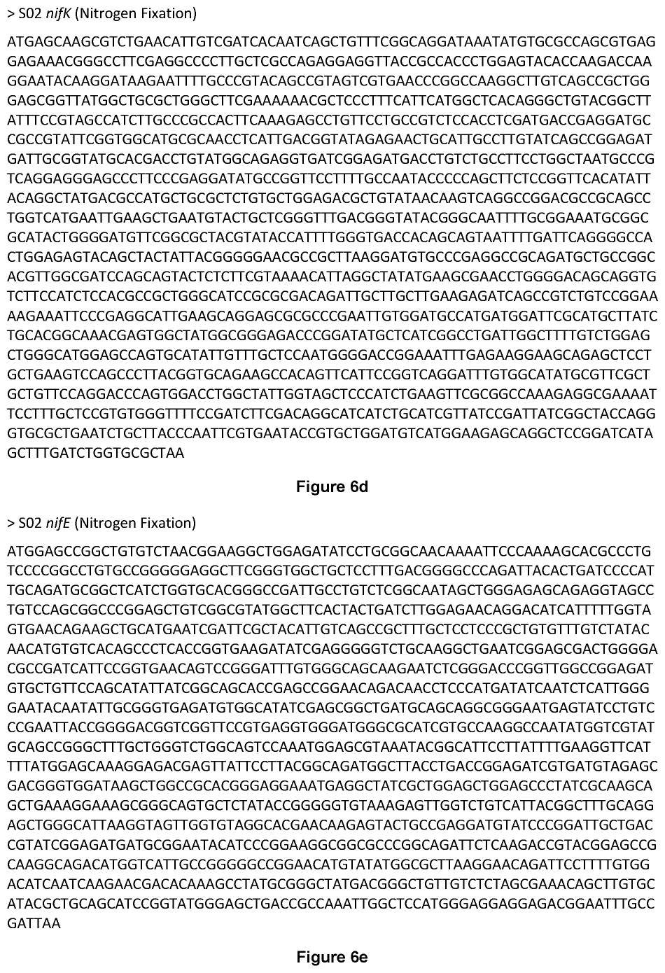

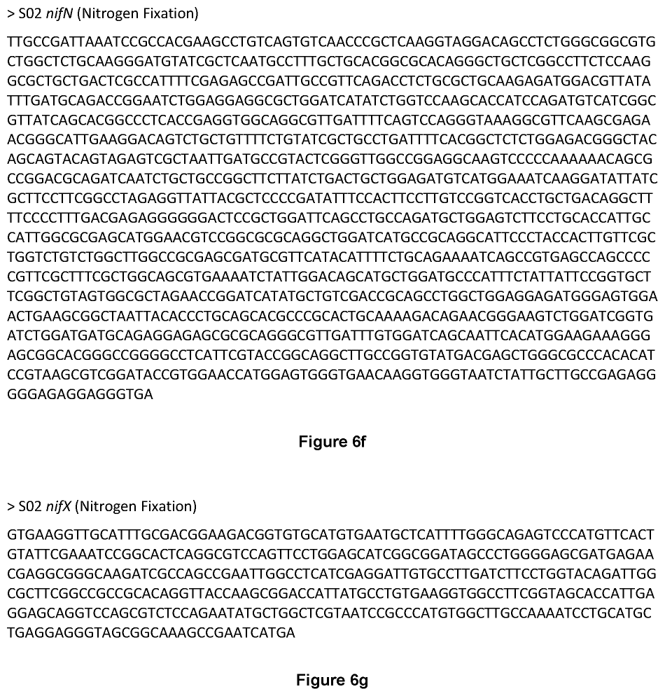

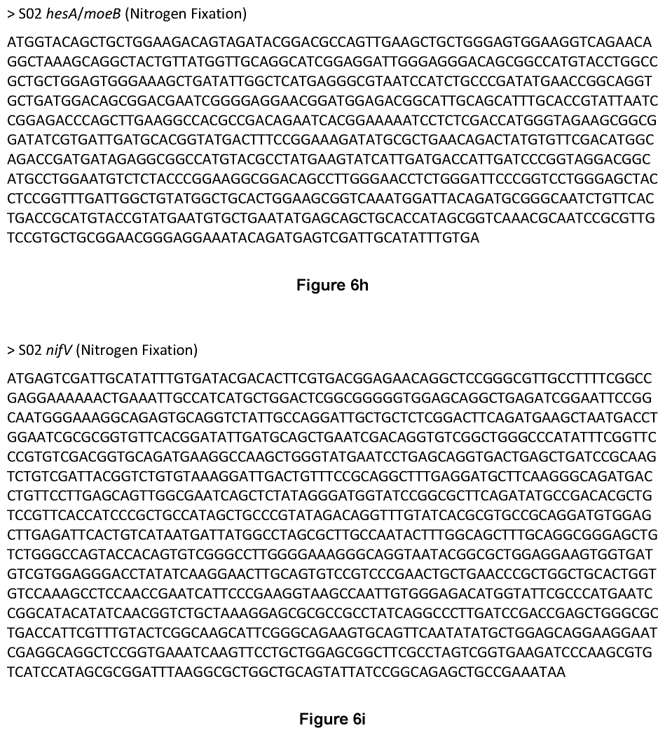

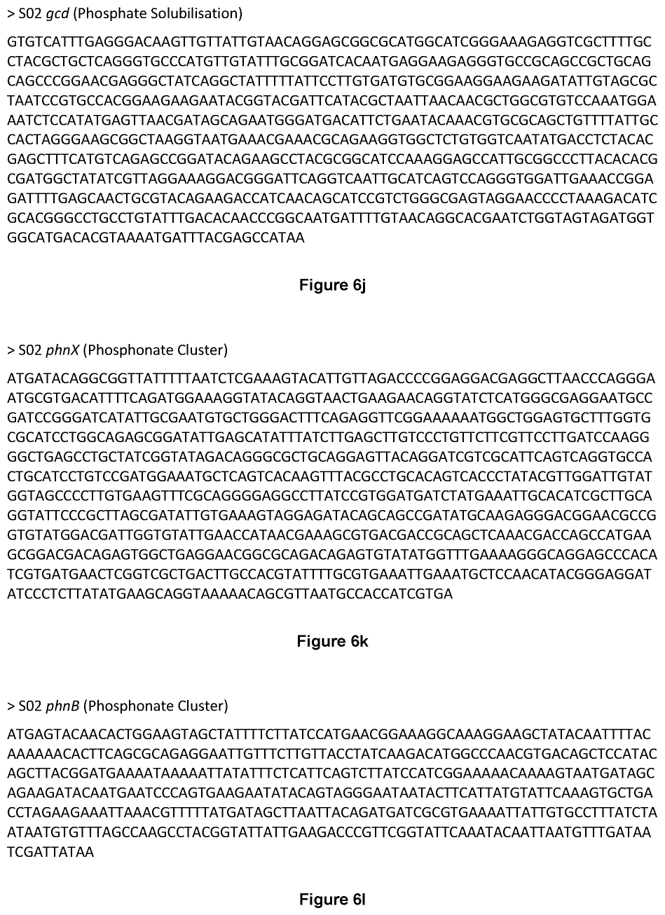

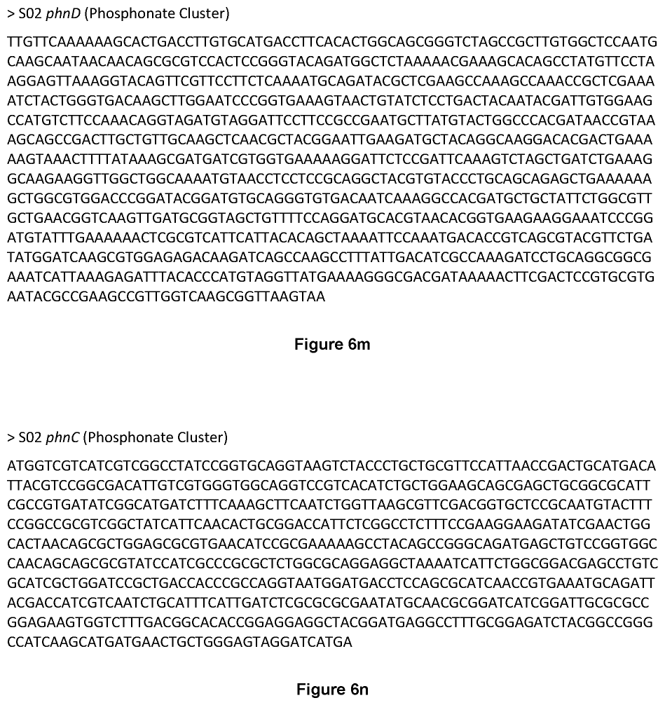

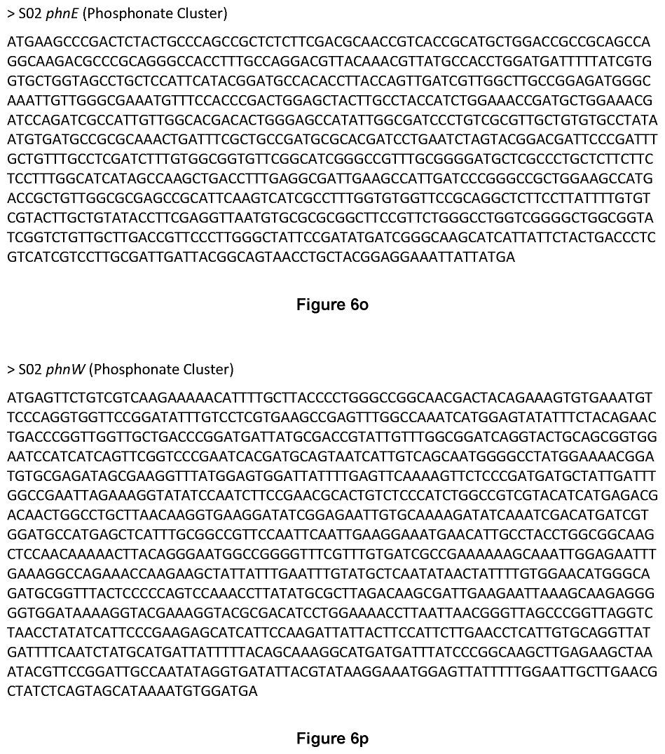

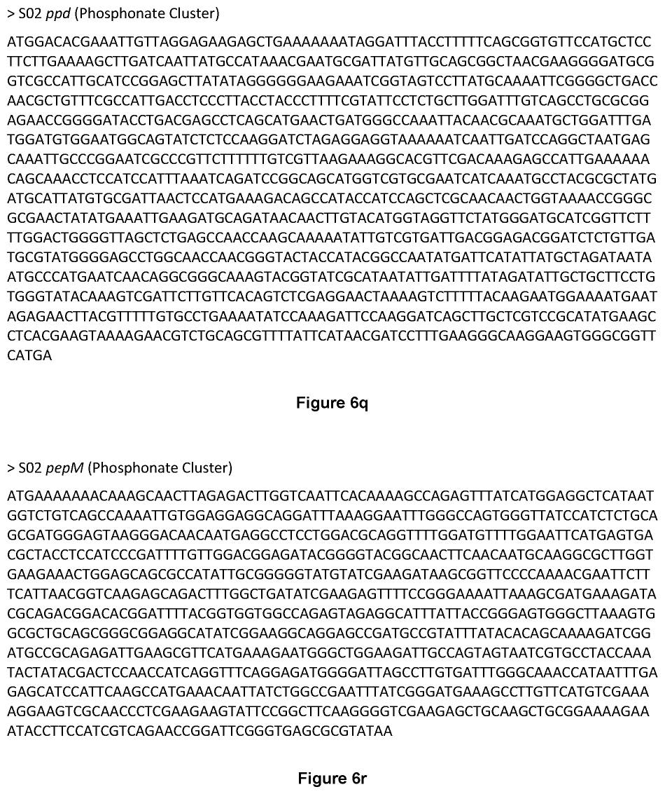

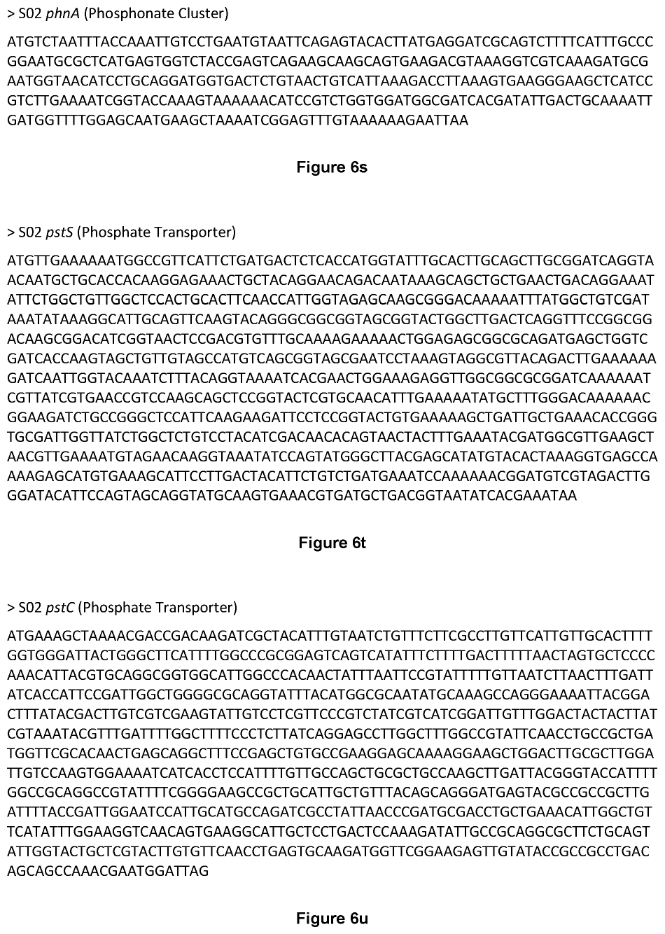

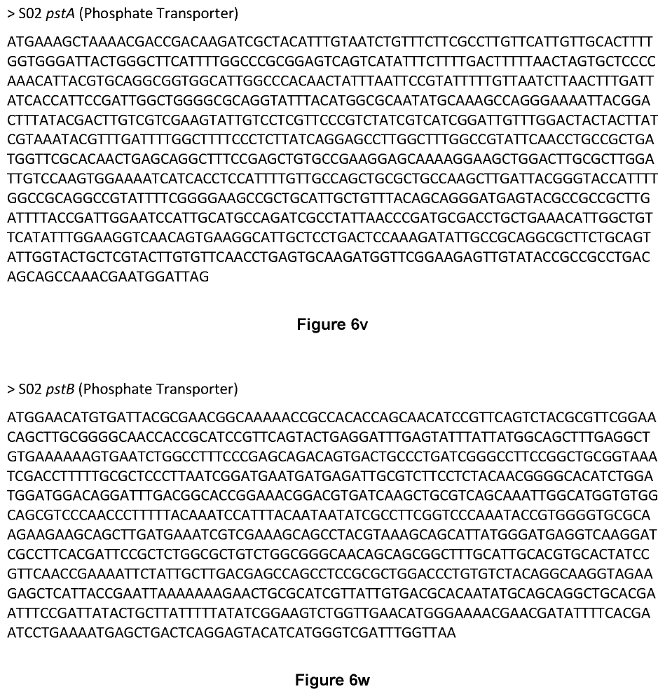

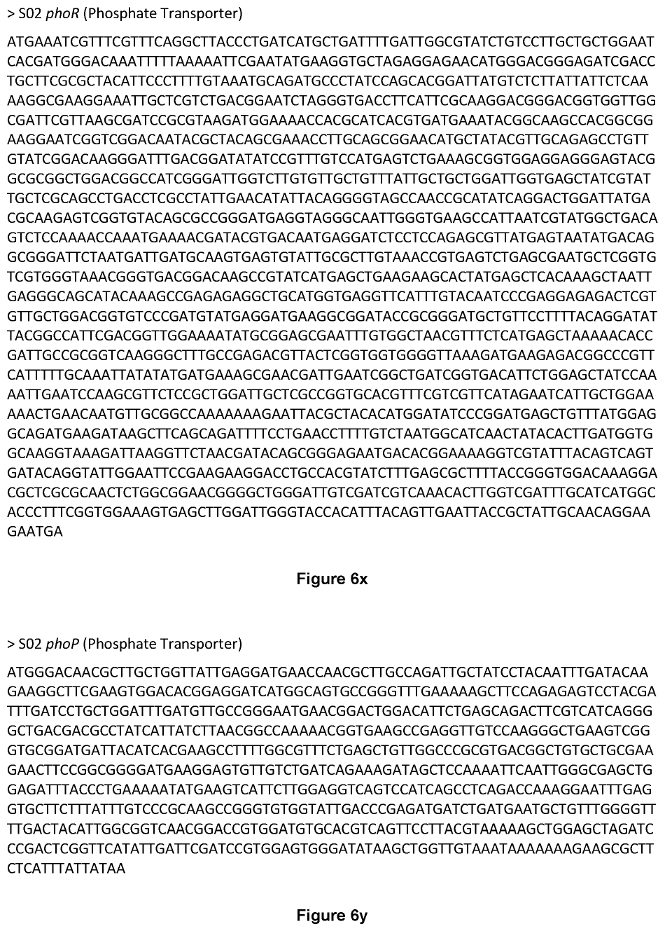

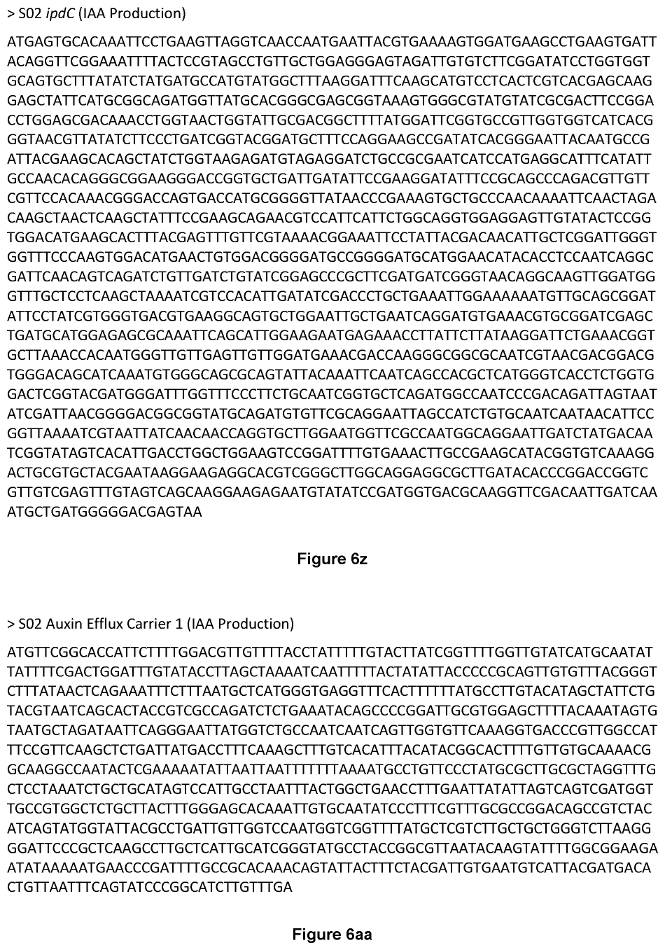

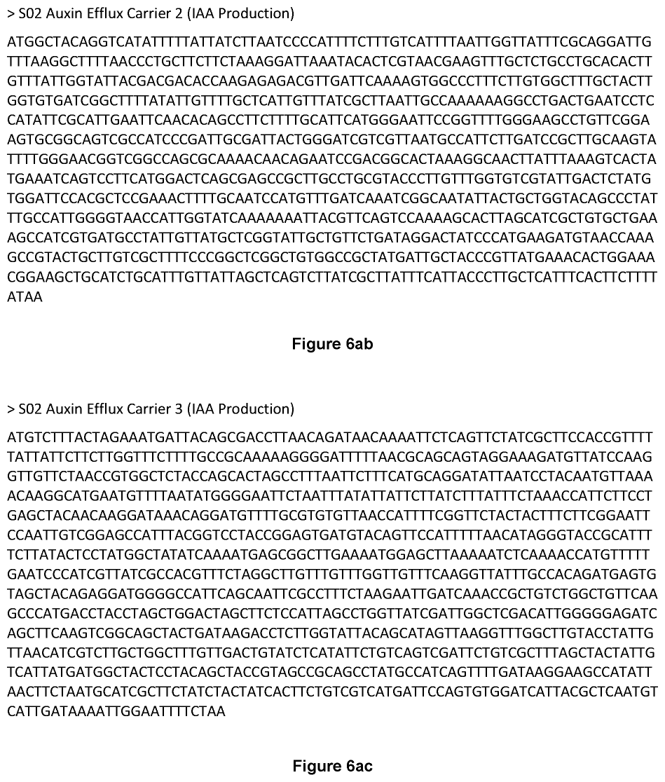

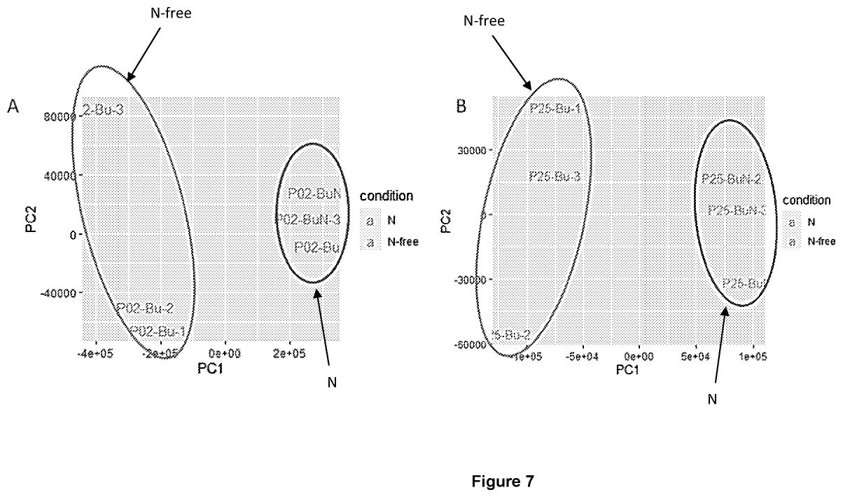

The genomes of both novel bacterial strains S02 and S25 were found to possess a complete set of PGP genes (Table 2). A 10.54 kb region containing a nif operon of nine genes (nifB/H/D/K/E/N/X, hesA/moeB and nifV) was identified which catalyses biological nitrogen fixation (Franche et al., 2009). In addition, 16 genes associated with phosphate solubilisation and assimilation were identified, including the glucose-1-dehydrogenase (gcd) gene for inorganic phosphate solubilisation (de Werra et al., 2009), the phn clusters of 9 genes for organic phosphate (phosphonates) solubilisation (Lugtenberg and Kamilova, 2009) and the phosphate-specific transport system of 6 genes for phosphate assimilation (Yuan et al., 2006). However, the gluconic acid dehydrogenase (gad) gene for inorganic phosphate solubilisation (de Werra et al., 2009) was not found in either of two strains. Additionally, genes involved in indole-3-acetic acid (IAA) production and transport were identified, including the ipdC gene that encodes a key enzyme in the IAA biosynthetic pathway (Spaepen et al., 2007), as well as three auxin efflux carrier genes. Sequence comparison of the PGP genes between novel bacterial strains S02 and S25 showed sequence similarity of 95.39-99.54%, while novel bacterial strains S02 and S25 showed sequencing similarity of 94.69-99.78% when compared to P. polymyxa strain CR1 (Table 2, FIGS. 6-7).

TABLE 2
Percent identify of 30 plant growth-promoting genes between the
genomes of novel strains S02 and S25 and P. polymyxa CR1

| | Percent Identity | | |
|---|---|---|---|
| | S02 VS S25 | S02 VS CR1 | S25 VS CR1 |
| Nitrogen fixation | | | |
| nifB | 97.13 | 98.00 | 96.80 |
| nifH | 95.39 | 98.15 | 94.69 |
| nifD | 98.96 | 98.62 | 98.27 |
| nifK | 98.24 | 97.45 | 97.39 |
| knife | 98.24 | 98.60 | 98.16 |
| nifN | 97.25 | 97.55 | 97.63 |
| nifX | 98.46 | 97.69 | 97.69 |
| hesA/moeB | 96.47 | 98.04 | 96.34 |
| nifV | 98.50 | 98.59 | 97.80 |
| Phosphate solubilization | | | |
| Gcd | 98.11 | 97.60 | 98.39 |
| Gad | — | — | — |
| Phosphonate cluster (phn) | | | |
| phnA | 98.53 | 98.53 | 98.82 |
| phnB | 98.21 | 98.43 | 99.78 |
| phnC | 97.66 | 97.81 | 98.39 |
| phnD | 98.14 | 99.07 | 97.63 |
| phone | 98.25 | 97.78 | 97.43 |
| phnW | 98.74 | 98.47 | 98.20 |
| phnX | 98.04 | 97.92 | 98.81 |
| Ppd | 98.97 | 98.62 | 98.79 |
| pepM | 99.33 | 99.44 | 99.00 |
| Phosphate transporter (pst) | | | |
| pstS | 98.92 | 98.48 | 99.13 |
| pstA | 96.99 | 97.55 | 98.22 |
| pstB | 98.81 | 98.70 | 98.22 |
| pstC | 99.14 | 98.71 | 98.82 |
| phoP | 98.09 | 97.81 | 97.94 |
| phoR | 98.28 | 97.45 | 97.89 |
| Indole-3-acetic acid | | | |
| production | | | |
| ipdC | 99.54 | 99.48 | 99.14 |
| auxin efflux carrier 1 | 99.13 | 98.15 | 98.37 |
| auxin efflux carrier 2 | 97.92 | 97.60 | 98.85 |
| auxin efflux carrier 3 | 98.58 | 98.96 | 99.05 |

### Example 4—Transcriptomic Evidence of Nif Gene Cluster Activity

Transcriptome sequencing experiments were designed to assess gene expression under different nitrogen treatments (+/− nitrogen), including the nif operon. Bacterial strains were cultured in Burk's N-free medium overnight (OD=1.0). Cultures were diluted using Burk's N-free medium to OD=0.7 and further cultured for 6 hours to produce actively growing cells for extracting high quality RNA (+N treatment). Burk's N-free medium supplemented with 10 g/L NH₄Cl was used for culturing the bacteria strains as the control (−N treatment). Three biological replicates were prepared for each treatment.

Total RNA was extracted form cell pellets using a TRIzol™ Plus RNA Purification Kit (12183555, Thermo Fisher Scientific). On-column treatments were conduct using a PureLink™ DNase (12185010, Thermo Fisher Scientific) to ensure the complete removal of genomic DNA that would affect the downstream analysis, and ribosomal RNA was depleted using a NEBNext® rRNA Depletion Kit (E7860L, NEB, Ipswich, MA, USA). Directional RNAseq libraries were prepared using a NEBNext Ultra™ II Directional RNA Library Prep Kit (E7765) and sequenced on an Illumina NovaSeq 6000 platform. RNAseq data (raw reads) were assessed for quality and filtered as per Example 2. An average of 13.8 million clean reads was generated per sample.

Salmon (Patro et al., 2017) was used to quantify transcripts with the following parameters: -I A --validateMappings --numBootstraps 1000 --seqBias. The references used for transcript quantification were the gene sequences of novel strain S02 and S25 (Example 2). A total of 1000 rounds of bootstraps were performed during transcript quantification to minimise the impact of technical variations. Differential gene expression (DGE) analysis was conducted using the R package sleuth (Pimentel et al., 2017). Principal Components Analysis (PCA) was performed to determine if biological replicates from the different treatments clustered separately, likelihood ratio tests (lrt) were conducted to detect the presence of any significant difference (q-vaule <0.05) in transcript abundance between treatments, and Wald tests were conducted to determine the fold-change in transcript abundance between treatments. Transcripts that were of ultra-low abundance (defined by having less than 20 mapped reads or were only present in less than 3 samples) were removed prior DGE analysis. The differentially expressed genes were defined to be significant at q-value <0.05 and absolute fold-change ≥1.5.

A total of 2467 (from 5059 that passed the abundance filter) and 2479 (from 4745) genes were differentially expressed when nitrogen was removed from the medium for novel strains S02 and S25, respectively. The nitrogen treatments (+/−) formed separate clusters following PCA, with replicates of both strains separating along the PC1, suggesting the presence/absence of nitrogen significantly affected the transcriptome of novel strains S02 and S25 (FIG. 7). Differential gene expression of the nif operon indicated that when nitrogen was absent from the media all nine genes were substantially up-regulated (8.62-22.50-fold change) in novel strain S02, compared to when nitrogen was present (Table 3). For novel strain S25, only five genes of the nif operon were up-regulated when nitrogen was absent compared to when it was present, with much more even gene expression observed (1.76-3.90-fold change). Results confirmed that both strains carry a transcriptionally active nif operon that may enable biological nitrogen fixation. Assessing the read counts of novel strains S02 and S25 under the two nitrogen treatments shows consistently low read counts for S25 across both nitrogen treatments (average: +N=14.9 reads, −N=26.4 reads), whereas S02 had low read counts in the +N treatment (average=19.44 reads) but high levels in the −N treatment (average=4=324.7) (Table 4).

TABLE 3
DGE of the nif operon of novel strains S02 and S25 when
nitrogen was absent from the medium, compared to when
it was present. The * indicates genes that were differentially
expressed (q-vaule < 0.05 and absolute fold-change ≥
1.5) when nitrogen was removed from the medium.

| | S02 | S25 |
|---|---|---|
| nif genes | fold change | fold change |
| nifB | 22.50* | 2.46* |
| nifH | 20.21* | 3.90* |
| nifD | 15.80* | 2.06* |
| nifK | 17.51* | 2.01* |
| knife | 15.86* | 1.76* |
| nifN | 18.16* | 1.59 |
| nifX | 8.62* | 1.19 |
| hesA/moeB | 15.13* | 1.56 |
| nifV | 11.01* | −1.46 |

TABLE 4
Read counts* for each replicate of novel bacteria S02 and S25 under the two nitrogen treatments (+/−Nitrogen).

| | S02 | | | | | | S25 | | | | | |
|---|---|---|---|---|---|---|---|---|---|---|---|---|
| | + Nitrogen | | | −Nitrogen | | | + Nitrogen | | | −Nitrogen | | |
| | Rep 1 | Rep 2 | Rep 3 | Rep 1 | Rep 2 | Rep 3 | Rep 1 | Rep 2 | Rep 3 | Rep 1 | Rep 2 | Rep 3 |
| nifB | 17.0 | 20.0 | 22.0 | 623.0 | 423.0 | 360.4 | 9.0 | 27.0 | 16.0 | 39.0 | 25.0 | 70.0 |
| nifH | 8.0 | 9.0 | 13.0 | 238.0 | 189.0 | 205.6 | 5.0 | 14.0 | 2.0 | 20.0 | 17.0 | 36.0 |
| nifD | 30.5 | 15.0 | 34.0 | 504.5 | 387.0 | 346.0 | 9.0 | 25.0 | 17.0 | 42.0 | 21.2 | 44.0 |
| nifK | 37.5 | 19.0 | 34.0 | 639.5 | 495.0 | 449.0 | 12.0 | 26.0 | 13.0 | 32.0 | 20.8 | 58.0 |
| nifE | 18.0 | 20.0 | 31.0 | 390.4 | 371.5 | 339.1 | 8.0 | 29.0 | 24.0 | 31.0 | 26.0 | 44.1 |
| nifN | 13.0 | 20.0 | 24.0 | 422.6 | 341.7 | 289.9 | 12.0 | 22.0 | 19.0 | 23.0 | 28.0 | 36.5 |
| nifX | 6.0 | 4.0 | 8.0 | 67.0 | 59.8 | 40.0 | 1.0 | 6.0 | 2.0 | 1.0 | 3.0 | 7.4 |
| hesA/moeB | 18.0 | 11.0 | 14.0 | 293.3 | 218.0 | 172.3 | 4.0 | 15.0 | 7.0 | 16.0 | 8.0 | 15.0 |
| nifV | 22.0 | 27.0 | 30.0 | 363.7 | 303.0 | 235.7 | 10.0 | 45.0 | 23.0 | 15.0 | 11.0 | 22.0 |

*Read counts are adjusted following 1000 rounds of bootstrapping

### Example 5—Bioprotection Bioassay

A bioassay was conducted to assess the in vitro bioprotection activity of novel strains S02 and S25 against three fungal pathogens of Poaceae species (Colletotrichum graminicola, Fusarium verticillioides and Microdochium nivale). The three fungal pathogens were obtained from the National Collection of Fungi (VPRI, Bundoora, Victoria, Australia). The design of in vitro bioassay was described in detail in Li et al. (2020). Briefly, the novel bacteria strains were drop-inoculated onto four equidistant points on a Nutrient Agar (BD Bioscience) plate, and pathogens were placed at the centre of the plate as a plug containing actively growing hyphae. The bioassay was incubated at 28° C. in the dark for 5 days. The diameter of the fungal colony was measured twice across 2 planes, and the average of the two readings was used for statistical analysis. Three plates were prepared for each treatment as biological replicates. Sterile medium was used as the blank control. Statistical analysis (One-way ANOVA and Tukey Test) was conducted using OriginPro 2020 (Version SR1 9.7.0.188) to detect the presence of any significant difference (P<0.05) between treatments.

Paenibacillus sp. strain S02 significantly (P<0.05) reduced the average colony diameter of the plant pathogens C. graminicola and F. verticillioides compared to the blank control and Paenibacillus sp. strain S25 (Table 5). It reduced the growth of C. graminicola and F. verticillioides by up to 74.9% and 56.9%, respectively. Paenibacillus sp. strain S25 significantly (P<0.05) reduced the growth of F. verticillioides by 9.6% compared to the blank control, however no biocidal activity was observed against C. graminicola. Neither of the two strains could significantly reduce the average colony diameter of M. nivale.

TABLE 5
The average colony diameter (±standard error) of
fungal pathogens when exposed to the two Paenibacillus
sp. strains in a bioprotection assay (in vitro)

| | | | Ave Colony Diameter (cm) | | |
|---|---|---|---|---|---|
| | | | of the Pathogen | | |
| Pathogen | VPRI | Host | S02 | S25 | Blank |
| Colletotrichum | 32315 | Cynosurus | 1.03 ± | 3.77 ± | 4.10 ± |
| graminicola | | echinatus | 0.02^{b} | 0.04^{a} | 0.20^{a} |
| Fusarium | 42586a | Zea mays L. | 2.93 ± | 6.15 ± | 6.80 ± |
| verticillioides | | | 0.04^{c} | 0.14^{b} | 0.08^{a} |
| Microdochium | 43403 | Lolium perenne | 5.12 ± | 5.18 ± | 5.27 ± |
| nivale | | | 0.11^{a} | 0.12^{a} | 0.04^{a} |

^{a, b, c}Letters represent statistical significance (P < 0.05).
Strain S02/S25: Paenibacillus sp. strains

### Example 6—Genome Sequence Features Supporting the Bioprotection Niche of the Novel Bacterial Strains S02 and S25

The presence of secondary metabolite gene clusters in the annotated genomes of S02 and S25 was assessed using antiSMASH (Weber et al., 2015) with the following options: --clusterblast --asf --knownclusterblast --subclusterblast --smcogs --full-hmmer. Secondary metabolite gene analysis identified 16 clusters (designated C1-C16) consisting 13 clusters that were shared by both strains and 3 clusters that only strain S25 possessed (Table 6). These additional clusters contain all the genes (core/additional biosynthetic genes, regulatory genes, transport-related genes and other genes) required for complete function. Secondary metabolite gene clusters that were shared by both strains included four that were identical to known clusters, including three nonribosomal peptide synthetase (Nrps) clusters (C1, fusaricidin B; 010, tridecaptin; C15, polymyxin) and one lanthipeptide cluster (C7, paenilan). The products of all four clusters have been reported to have antimicrobial bioactivities (Li and Jensen, 2008; Choi et al., 2009; Lohans et al., 2014; Park et al., 2017). Other clusters that matched the antiSMASH database based on sequence homology included a lassopeptide cluster (C5), a Nrps cluster (C6), a Nrps/transAT-polyketide synthase (PKS) cluster (C11) and a Nrps/Type III (T3) PKS/transAT-PKS cluster (C14). Among these four clusters, cluster C11 had the highest similarity (S02, 73%; S25, 76%) to a known cluster of P. polymyxa E681 that produces paenilipoheptin (Vater et al., 2018). There were a further five clusters that appear novel based on sequence homology to the antiSMASH database, including one siderophore cluster (C2), one bacteriocin cluster (C3), one Nrps/transAT-PKS cluster (C4), one Nrps-like cluster (C9) and one phosphonate cluster (C16). Paenibacillus sp. strain S25 had three unique secondary metabolite gene clusters that were missing from the genome of Paenibacillus sp. strain S02, including a lanthipeptide cluster (C8), and two novel Nrps clusters (C12, C13). While the two Nrps appear novel based on sequence homology, the lanthipeptide cluster had a similarity of 71% to a known paenicidin B cluster, which was a novel lantibiotic active against Gram-positive bacteria produced by Paenibacillus terrae (Lohans et al., 2014). Overall, both novel strains had a diverse array of secondary metabolite gene clusters that support its bioprotection niche.

TABLE 6
Secondary metabolite gene clusters identified in strain Paenibacillus sp. strain S02 and S25

| | | Location | | |
|---|---|---|---|---|
| ID | Type | Paenibacillus sp. S02 | Paenibacillus sp. S25 | Most similar known cluster (similarity) |
| C1 | Nrps | 62,712-130,949 | 62,863-131,149 | fusaricidin B(100%) |
| C2 | siderophore | 1,060,830-1,078,231 | 1,021,525-1,038,926 | N/A |
| C3 | bacteriocin | 1,226,685-1,236,921 | 1,163,825-1,174,061 | N/A |
| C4 | Nrps; transAT-PKS | 1,276,170-1,374,849 | 1,234,262-1,333,113 | N/A |
| C5 | lassopeptide | 1,410,732-1,434,848 | 1,369,083-1,393,199 | paeninodin (40%) |
| C6 | Nrps | 1,496,857-1,557,694 | 1,452,621-1,513,241 | marthiapeptide A (33%) |
| C7 | lanthipeptide | 1,752,471-1,779,477 | 1,717,117-1,742,305 | paenilan(100%) |
| C8 | lanthipeptide | N/A | 1,865,337-1,891,786 | paenicidin B (71%) |
| C9 | Nrps-like | 2,147,919-2,191,265 | 2,068,326-2,110,857 | N/A |
| C10 | Nrps | 2,564,994-2,657,512 | 2,538,234-2,631,151 | tridecaptin(100%) |
| C11 | Nrps; transAT-PKS | 2,800,573-2,881,430 | 2,762,852-2,843,624 | paenilipoheptin (S02, 73%; S25, 76%) |
| C12 | Nrps | N/A | 2,847,977-2,929,143 | N/A |
| C13 | Nrps; betalactone | N/A | 3,004,139-3,056,727 | N/A |
| C14 | Nrps; T3PKS; transAT- | 3,755,116-3,856,856 | 3,756,437-3,858,120 | aurantinin B/C/D (35%) |
| | PKS | | | |
| C15 | Nrps | 5,189,939-5,270,981 | 5,092,876-5,173,931 | polymyxin(100%) |
| C16 | phosphonate | 5,879,383-5,920,282 | 5,775,191-5,816,090 | N/A |

Nrps: nonribosomal peptide synthetase
transAT-PKS: transAT-polyketide synthase
T3PKS: Type III polyketide synthase
Clusters in bold: known antimicrobial compounds;

### Example 7—Transcriptomics Evidence of Bioprotection Activity

Transcriptome sequencing experiments were designed to assess gene expression under different pathogen treatments (+/−Fusarium verticillioides), including the 16 secondary metabolite gene clusters of novel strains S02 and S25. Bacterial strains and plant pathogenic fungus Fusarium verticillioides (VPRI42586a) were cultured in Nutrient Broth (NB) overnight (bacteria: OD=1.0). Bacterial cultures were diluted using NB to OD=0.7 and 20 mL of the culture was mixed with 200 μL of the pathogen culture and was further incubated for 6 hours. For the control, the pathogen culture was replaced by sterile NB. Three biological replicates were prepared for each treatment. Total RNA extractions, library preparation and sequencing were prepared as per Example 4. RNAseq data (raw reads) were assessed for quality and filtered as per Example 2. An average of 31.1 million clean reads was generated per sample. Transcript quantification, DGE and statistical analysis were conducted as per Example 4.

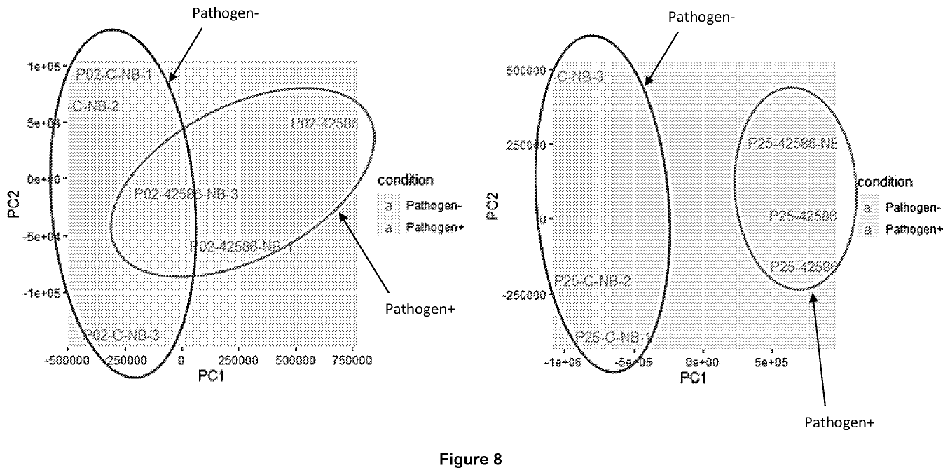

A total of 61 (from 5201 that passed the abundance filter) and 2706 (from 4817) genes were differentially expressed when the pathogen was present for novel strains S02 and S25, respectively. The pathogen treatments (+/−) formed separate clusters following PCA, with replicates of both strains separating along the PC1, although the separation was clearest for S25. The data suggests the presence/absence of pathogen significantly affected the transcriptome of novel strains S02 and S25 (FIG. 8).

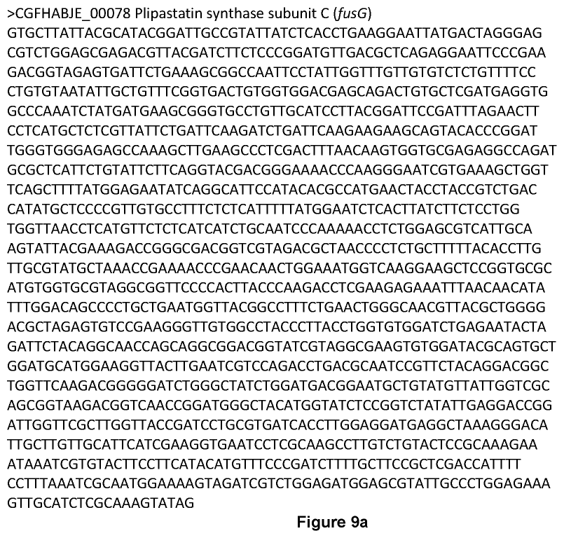

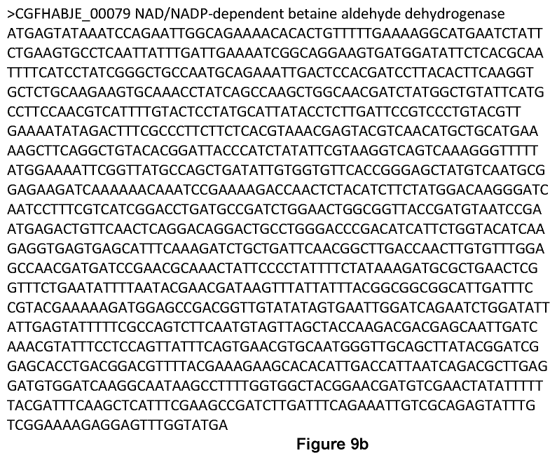

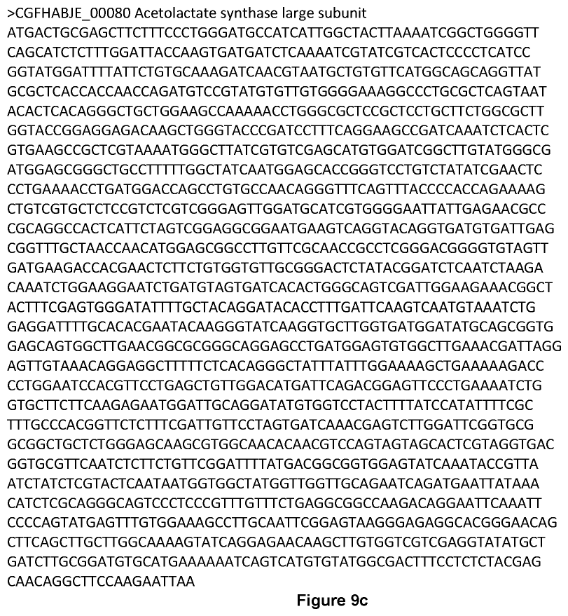

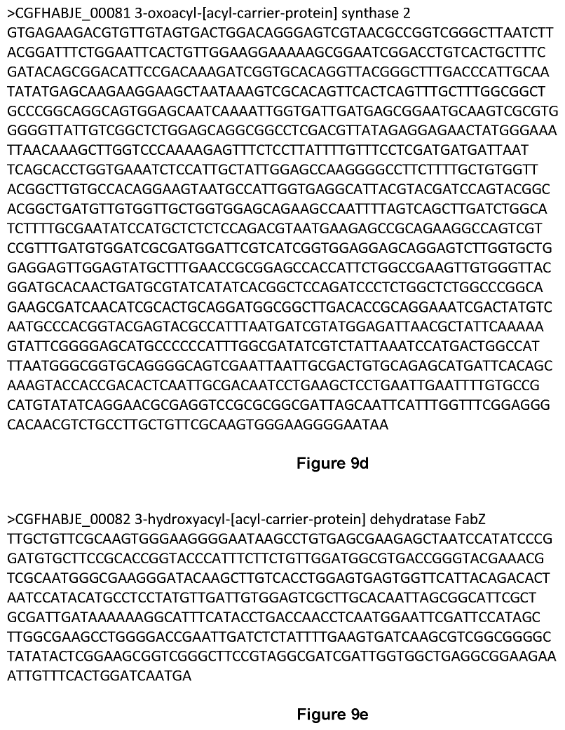

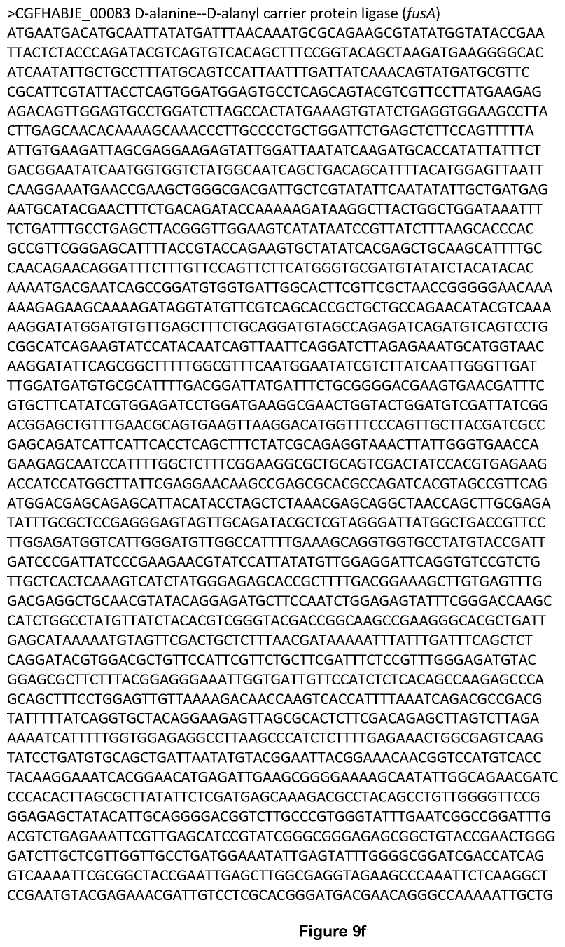

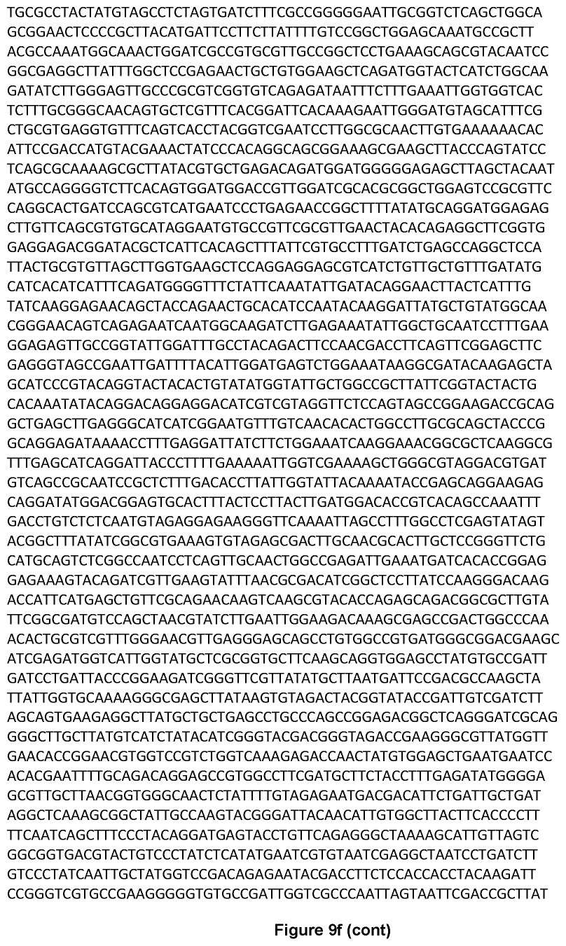

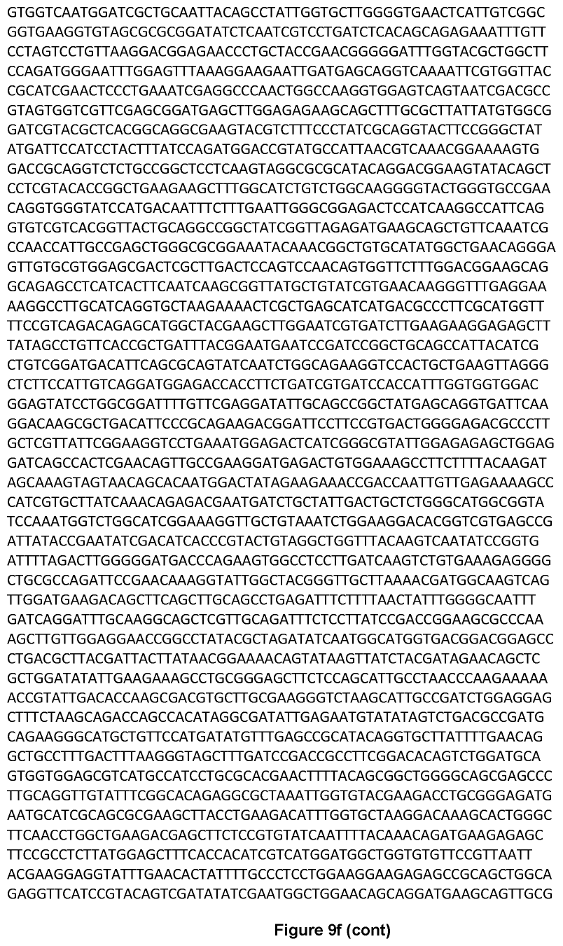

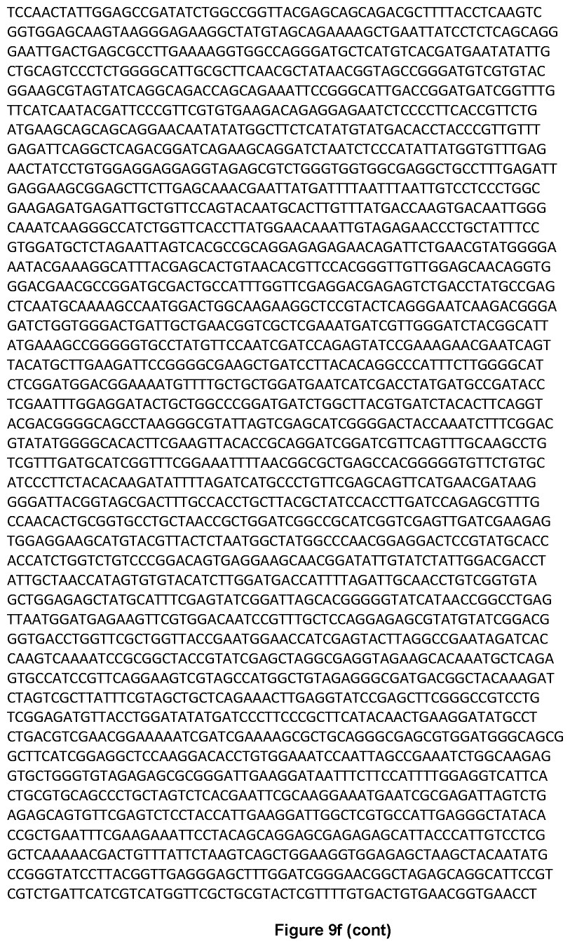

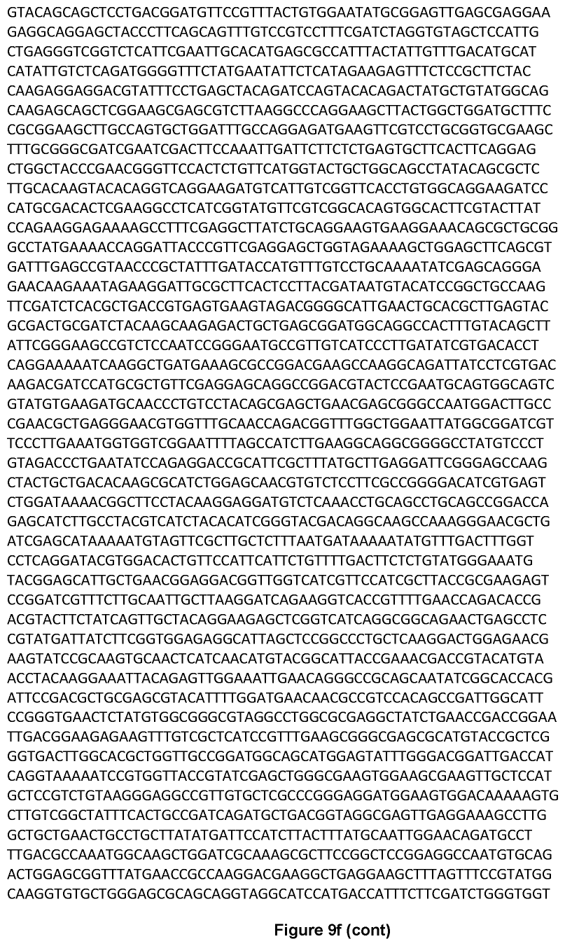

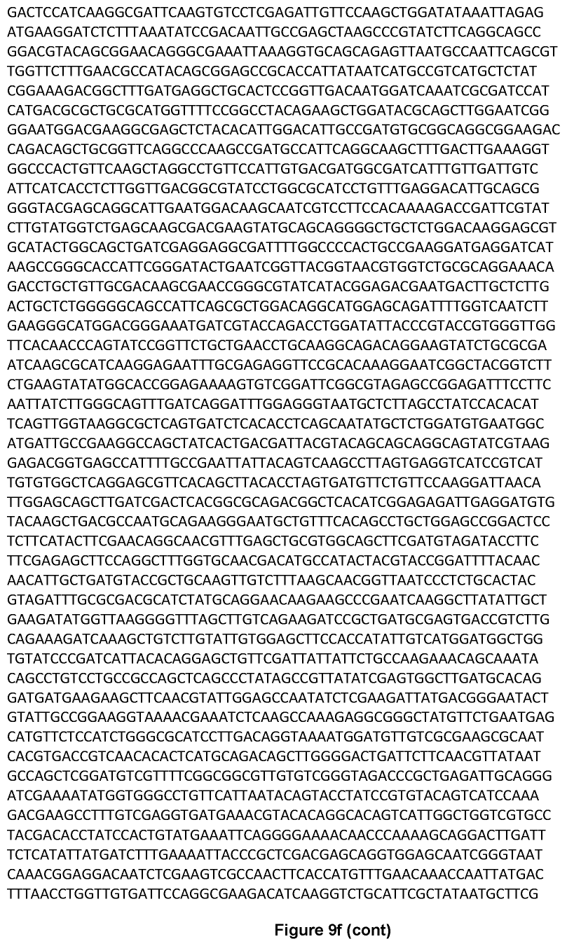

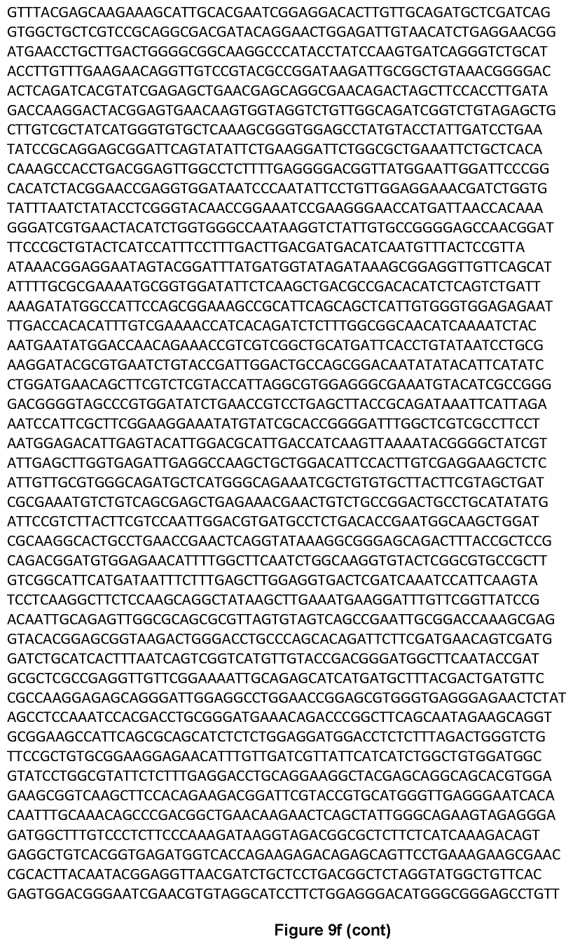

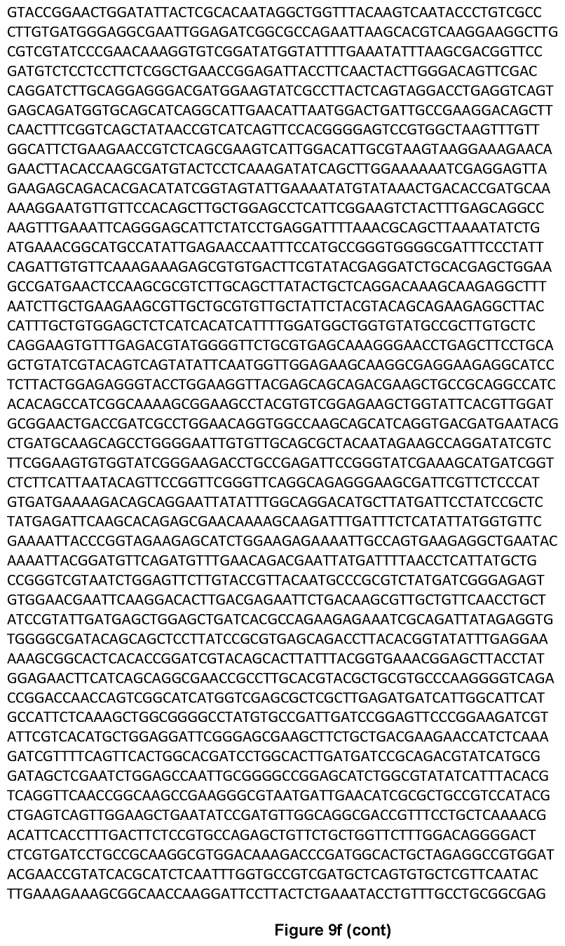

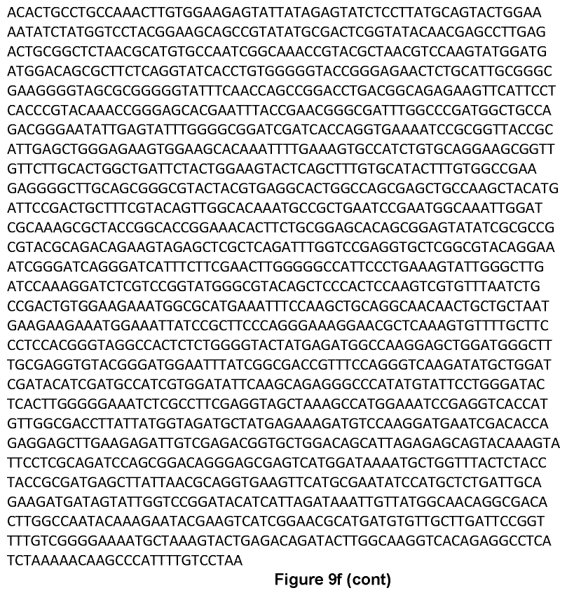

Differential gene expression of the core biosynthetic genes of secondary metabolite gene clusters indicated that when the pathogen was absent from the media the majority of clusters in novel strain S02 were differentially expressed (42 or 44), compared to novel strain S25 (Table 7). Of these, 41 genes were up-regulated and one was down-regulated. For the genes that were up-regulated the fold change ranged from 1.92-486.58 (average fold increase=62.80), while the gene that was down-regulated had a 7.42 fold change. When the pathogen was present, there were only 3 of 44 genes that were differentially expressed for novel strain S02, and 31 of 44 for novel strain S25 (Table 8). For novel strain S02, there were two genes that were down-regulated (range=1.65-1.89) and one gene that was up-regulated (1.81). For novel strain S25 there were 16 genes that were down-regulated (range=1.08-5.21; average=2.41) and 15 genes that were up-regulated (range=1.50-23.42; average=5.64). Overall, novel strain S02 showed high expression levels of most genes, irrespective of whether the pathogen was present or not, compared to novel strain S25. The secondary metabolite gene clusters that had the highest up-regulation (>50 fold increase) were fusaricidin B (C1), paeninodin (C5), marthiapeptide A (C6), paenilan (C7) and aurantinin B/C/D (C14), which may relate to the increased bioactivity seen in novel strain S02, compared to novel strain S25. Of these five gene clusters, the one associated with fusaricidin B is of greatest interest as it has been shown to have activity against Fusarium spp., as well as a range of other important crop pathogens including Verticillium albo-atrum, Monilia persoon, Alternaria mali, Botrytis cinerea, and Aspergillus niger (FIG. 9) (Li and Chen 2019). The cluster associated with paenilan (C7) production are of less interest as they are more associated with anti-bacterial activity (Park et al. 2017), while the cluster associated with paeninodin (C5) has been shown to have limited bioactivity against bacteria (Zhu et al 2016). The clusters associated with marthiapeptide A (C6) and aurantinin B/C/D (C14) only have low sequence homology so they are unlikely to produce these compounds, and so their activity is unknown.

TABLE 7
DGE of the core biosynthetic genes of secondary metabolite gene clusters
of novel Paenibacillus sp. strains S02 compared to S25 when the
pathogen was absent from the medium. The * indicates genes that
were differentially expressed (q-value < 0.05 and absolute
fold-change ≥ 1.5) when nitrogen was removed from the medium.

| | | Most similar known | Paenibacillus sp. S02 | |
|---|---|---|---|---|
| | | cluster | | Fold |
| ID | Type | (% similarity) | Gene ID | change |
| C1 | Nrps | fusaricidin B(100%) | CGFHABJE_00078 | 486.58* |
| | | | CGFHABJE_00083 | 20.36* |
| C2 | siderophore | N/A | CGFHABJE_00955 | 11.71* |
| | | | CGFHABJE_00956 | 13.93* |
| | | | CGFHABJE_00959 | 4.64* |
| C3 | bacteriocin | N/A | CGFHABJE_01103 | 46.95* |
| C4 | Nrps | N/A | CGFHABJE_01166 | 18.88* |
| | transAT- | | CGFHABJE_01170 | 2.43* |
| | PKS | | CGFHABJE_01172 | 1.93* |
| | | | CGFHABJE_01173 | 3.31* |
| | | | CGFHABJE_01175 | 2.35* |
| | | | CGFHABJE_01176 | 2.30* |
| | | | CGFHABJE_01178 | 2.27* |
| | | | CGFHABJE_01179 | 1.92* |
| | | | CGFHABJE_01180 | −1.25 |
| | | | CGFHABJE_01181 | 1.27 |
| C5 | lassopeptide | paeninodin (40%) | CGFHABJE_01236 | 134.38* |
| | | | CGFHABJE_01240 | 37.82* |
| C6 | Nrps | marthiapeptide | CGFHABJE_01339 | 239.45* |
| | | A (33%) | CGFHABJE_01340 | 159.93* |
| | | | CGFHABJE_01341 | 119.29* |
| C7 | lanthipeptide | paenilan(100%) | CGFHABJE_01558 | 60.94* |
| | | | CGFHABJE_01560 | 271.12* |
| | | | CGFHABJE_01562 | 69.08* |
| C9 | Nrps-like | N/A | CGFHABJE_01944 | −7.42* |
| C10 | Nrps | tridecaptin(100%) | CGFHABJE_02333 | 7.74* |
| | | | CGFHABJE_02334 | 32.36* |
| C11 | Nrps | paenilipoheptin | CGFHABJE_02506 | 17.20* |
| | transAT- | (S02, 73%; | CGFHABJE_02507 | 29.10* |
| | PKS | S25, 76%) | CGFHABJE_02508 | 23.54* |
| | | | CGFHABJE_02509 | 22.65* |
| | | | CGFHABJE_02510 | 10.63* |
| C14 | Nrps | aurantinin | CGFHABJE_03362 | 118.38* |
| | T3PKS | B/C/D (35%) | CGFHABJE_03363 | 46.83* |
| | transAT- | | CGFHABJE_03365 | 125.65* |
| | PKS | | CGFHABJE_03366 | 98.69* |
| | | | CGFHABJE_03367 | 31.92* |
| | | | CGFHABJE_03368 | 77.19* |
| | | | CGFHABJE_03371 | 134.46* |
| | | | CGFHABJE_03372 | 113.90* |
| C15 | Nrps | polymyxin(100%) | CGFHABJE_04684 | 9.61* |
| | | | CGFHABJE_04687 | 8.61* |
| | | | CGFHABJE_04688 | 9.86* |
| C16 | phosphonate | N/A | CGFHABJE_05277 | 15.10* |

Clusters in bold: known antimicrobial compounds
*genes that were differentially expressed (q-value < 0.05 and absolute fold-change ≥ 1.5) when comparing the two strains.

TABLE 8
DGE of the core biosynthetic genes of secondary metabolite gene clusters of novel
Paenibacillus sp. strains S02 and S25 when the pathogen was present in the medium,
compared to when it was absent. The * indicates genes that were differentially expressed
(q-vaule < 0.05 and absolute fold-change ≥ 1.5) when nitrogen was removed from the medium.

| | | Most similar | Paenibacillus sp. S02 | | Paenibacillus sp. S25 | |
|---|---|---|---|---|---|---|
| | | known cluster | | Fold | | Fold |
| ID | Type | (% similarity) | Gene ID | change | Gene ID | change |
| C1 | Nrps | fusaricidin B | CGFHABJE_ | −1.28 | KKIAGPJH_ | −1.50 |
| | | (100%) | 00078 | | 00078 | |
| | | | CGFHABJE_ | −1.36 | KKIAGPJH_ | −5.21* |
| | | | 00083 | | 00083 | |
| C2 | siderophore | N/A | CGFHABJE_ | −1.23 | KKIAGPJH_ | 2.06* |
| | | | 00955 | | 00927 | |
| | | | CGFHABJE_ | −1.15 | KKIAGPJH_ | 2.60* |
| | | | 00956 | | 00928 | |
| | | | CGFHABJE_ | −1.12 | KKIAGPJH_ | 1.04 |
| | | | 00959 | | 00931 | |
| C3 | bacteriocin | N/A | CGFHABJE_ | 1.10 | KKIAGPJH_ | −1.19 |
| | | | 01103 | | 01049 | |
| C4 | Nrps | N/A | CGFHABJE_ | −1.25 | KKIAGPJH_ | −1.17 |
| | transAT-PKS | | 01166 | | 01130 | |
| | | | CGFHABJE_ | −1.89* | KKIAGPJH_ | −1.08 |
| | | | 01170 | | 01134 | |
| | | | CGFHABJE_ | −1.54 | KKIAGPJH_ | −1.43 |
| | | | 01172 | | 01136 | |
| | | | CGFHABJE_ | −1.65* | KKIAGPJH_ | −1.49 |
| | | | 01173 | | 01137 | |
| | | | CGFHABJE_ | −1.64 | KKIAGPJH_ | −1.52 |
| | | | 01175 | | 01139 | |
| | | | CGFHABJE_ | −1.48 | KKIAGPJH_ | −1.56 |
| | | | 01176 | | 01140 | |
| | | | CGFHABJE_ | −1.41 | KKIAGPJH_ | −1.51* |
| | | | 01178 | | 01142 | |
| | | | CGFHABJE_ | −1.40 | KKIAGPJH_ | −1.55 |
| | | | 01179 | | 01143 | |
| | | | CGFHABJE_ | −1.43 | KKIAGPJH_ | −2.34* |
| | | | 01180 | | 01144 | |
| | | | CGFHABJE_ | −1.33 | KKIAGPJH_ | −1.90* |
| | | | 01181 | | 01145 | |
| C5 | Iassopeptide | paeninodin | CGFHABJE_ | −1.20 | KKIAGPJH_ | −3.63* |
| | | (40%) | 01236 | | 01200 | |
| | | | CGFHABJE_ | −1.30 | KKIAGPJH_ | −2.07 |
| | | | 01240 | | 01204 | |
| C6 | Nrps | marthiapeptide A | CGFHABJE_ | 1.19 | KKIAGPJH_ | 4.34* |
| | | (33%) | 01339 | | 01293 | |
| | | | CGFHABJE_ | 1.15 | KKIAGPJH_ | 2.43* |
| | | | 01340 | | 01294 | |
| | | | CGFHABJE_ | 1.12 | KKIAGPJH_ | 2.02* |
| | | | 01341 | | 01295 | |
| C7 | lanthipeptide | paenilan | CGFHABJE_ | −1.04 | KKIAGPJH_ | 1.32 |
| | | (100%) | 01558 | | 01518 | |
| | | | CGFHABJE_ | −1.00 | KKIAGPJH_ | 1.10 |
| | | | 01560 | | 01520 | |
| | | | CGFHABJE_ | 1.02 | KKIAGPJH_ | 1.18 |
| | | | 01562 | | 01522 | |
| C8 | lanthipeptide | paenicidin B | | | KKIAGPJH_ | −1.73 |
| | | (71%) | | | 01661 | |
| | | | | | KKIAGPJH | −1.19 |
| | | | | | 01663 | |
| C9 | Nrps-like | N/A | CGFHABJE_ | 1.81* | KKIAGPJH_ | 2.07* |
| | | | 01944 | | 01854 | |
| C10 | Nrps | tridecaptin | CGFHABJE_ | 1.02 | KKIAGPJH_ | −1.92* |
| | | (100%) | 02333 | | 02322 | |
| | | | CGFHABJE_ | 1.00 | KKIAGPJH_ | 1.79* |
| | | | 02334 | | 02323 | |
| C11 | Nrps | paenilipoheptin | CGFHABJE_ | 1.14 | KKIAGPJH_ | 15.43* |
| | transAT-PKS | (S02, 73%; | 02506 | | 02476 | |
| | | S25, 76%) | CGFHABJE_ | 1.16 | KKIAGPJH_ | 23.42* |
| | | | 02507 | | 02477 | |
| | | | CGFHABJE_ | 1.16 | KKIAGPJH_ | 11.77* |
| | | | 02508 | | 02478 | |
| | | | CGFHABJE_ | 1.21 | KKIAGPJH_ | 7.92* |
| | | | 02509 | | 02479 | |
| | | | CGFHABJE_ | 1.11 | KKIAGPJH_ | 3.23* |
| | | | 02510 | | 02480 | |
| C12 | Nrps | N/A | | | KKIAGPJH_ | −1.91* |
| | | | | | 02516 | |
| C13 | Nrps | N/A | | | KKIAGPJH_ | 1.50* |
| | betalactone | | | | 02623 | |
| | | | | | KKIAGPJH_ | 2.02* |
| | | | | | 02624 | |
| | | | | | KKIAGPJH_ | 2.10* |
| | | | | | 02633 | |
| C14 | Nrps | aurantinin B/C/D | CGFHABJE_ | −1.29 | KKIAGPJH_ | 1.71 |
| | T3PKS | (35%) | 03362 | | 03372 | |
| | transAT-PKS | | CGFHABJE_ | −1.35 | KKIAGPJH_ | −1.78 |
| | | | 03363 | | 03373 | |
| | | | CGFHABJE_ | −1.32 | KKIAGPJH_ | −1.66* |
| | | | 03365 | | 03375 | |
| | | | CGFHABJE_ | −1.34 | KKIAGPJH_ | −1.82* |
| | | | 03366 | | 03376 | |
| | | | CGFHABJE_ | −1.26 | KKIAGPJH_ | −2.00* |
| | | | 03367 | | 03377 | |
| | | | CGFHABJE_ | −1.05 | KKIAGPJH_ | −1.78* |
| | | | 03368 | | 03378 | |
| | | | CGFHABJE_ | −1.05 | KKIAGPJH_ | N/A |
| | | | 03371 | | 03381 | |
| | | | CGFHABJE_ | −1.03 | KKIAGPJH_ | −4.68* |
| | | | 03372 | | 03382 | |
| C15 | Nrps | polymyxin | CGFHABJE_ | −1.02 | KKIAGPJH_ | −2.61* |
| | | (100%) | 04684 | | 04566 | |
| | | | | | KKIAGPJH_ | −2.33* |
| | | | | | 04567 | |
| | | | CGFHABJE_ | 1.10 | KKIAGPJH_ | −1.45* |
| | | | 04687 | | 04570 | |
| | | | CGFHABJE_ | 1.12 | KKIAGPJH_ | −1.92* |
| | | | 04688 | | 04571 | |
| C16 | phosphonate | N/A | CGFHABJE_ | −1.38 | KKIAGPJH_ | −1.77* |
| | | | 05277 | | 05154 | |

Clusters in bold: known antimicrobial compounds
*genes that were differentially expressed (q-value < 0.05 and absolute fold-change ≥ 1.5) when comparing the two strains.

### Example 5—Transcriptomics Evidence of Symbiotic Interaction with the Plant

A transcriptome sequencing experiment was designed to assess gene expression in early stage plant-bacteria interactions. Barley (Hordeum vulgare, variety Hindmarsh) seeds and three bacterial strains isolated from the perennial ryegrass (Lolium perenne L. cv. Alto) microbiome (Tannenbaum et al., 2020) were used in this assay, including two novel Paenibacillus sp. strains (S02 and S25) and one novel E. gerundensis strain (AR). Two media were utilised as the substrates for the assay, with either a standard bacterial media (Nutrient Broth, NB) or a nitrogen-free media (Burk's media, S02 only).

Barley seeds were surface-sterilised (80% ethanol for 3 minutes, followed by 3× sterile dH₂O washes) and germinated under sterile conditions (on moistened sterile filter paper in a sealed Petri dish). All bacterial strains were cultured overnight (OD₆₀₀=1.0) in Nutrient Broth (NB, BD Bioscience), while S02 was also cultured overnight in Burk's N-free medium (MgSO₄, 0.2 g/L; K₂HPO₄, 0.8 g/L; KH₂PO₄, 0.2 g/L; CaSO₄, 0.13 g/L; FeCl₃, 0.00145 g/L; Na₂MoO₄, 0.000253 g/L; sucrose, 20 g/L) and were diluted using fresh media to OD₆₀₀=0.7 (final volume=50 mL). Seedlings (5 days old) had their roots submerged in the bacterial culture, and incubated at 26° C. for 6 hours, with shaking (100 rpm). For the blank control (seedling only), seedlings had their roots submerged in sterile medium (NB or Burk's N-free medium) without the presence of bacteria. For the blank control (bacteria), bacteria were cultured without the presence of a seedling. Three samples were prepared as biological replicates for each treatment and control. Bacterial cells and plant root tissues were harvested after 6 hours of co-incubation and were used for RNA extraction immediately. Total RNA extractions, library preparation and sequencing were prepared as per Example 4. RNAseq data (raw reads) were assessed for quality and filtered as per Example 2. An average of 11.7 million clean reads was generated per sample. Transcript quantification, DGE and statistical analysis were conducted as per Example 4. For the bacterial samples, genes for novel bacterial species S02 (5436 genes) S25 (5306 genes) and AR (4091 genes) were used. For the plant samples, a barley reference transcript dataset (BaRTv1.0) containing 60444 genes with 177240 transcripts was used (Rapazote-Flores et al. 2019).

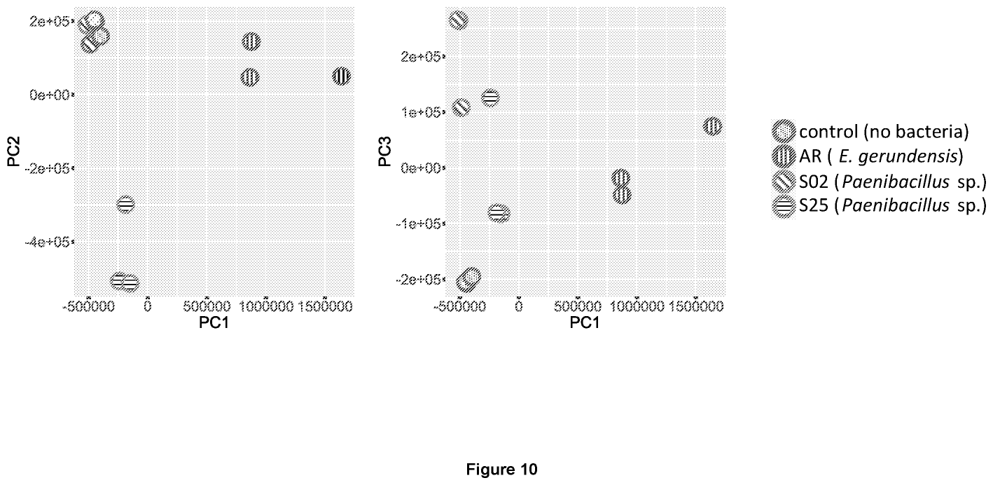

When comparing barley seedlings co-incubated with novel bacterial strains in NB, seedlings co-incubated with strain AR or S25 formed distinct clusters separate from the control seedlings along all three axes (PC1-PC3, FIG. 10). Conversely, seedlings co-incubated with strain S02 formed a cluster with the control seedlings along PC1 and PC2, only separating along PC3 which accounted for less than 5% of the total variances. These results suggested strain S02 produces transcriptome profiles in barley seedling similar to the uninoculated control, unlike strains AR and S25 which produce distinct transcriptome profiles in barley.

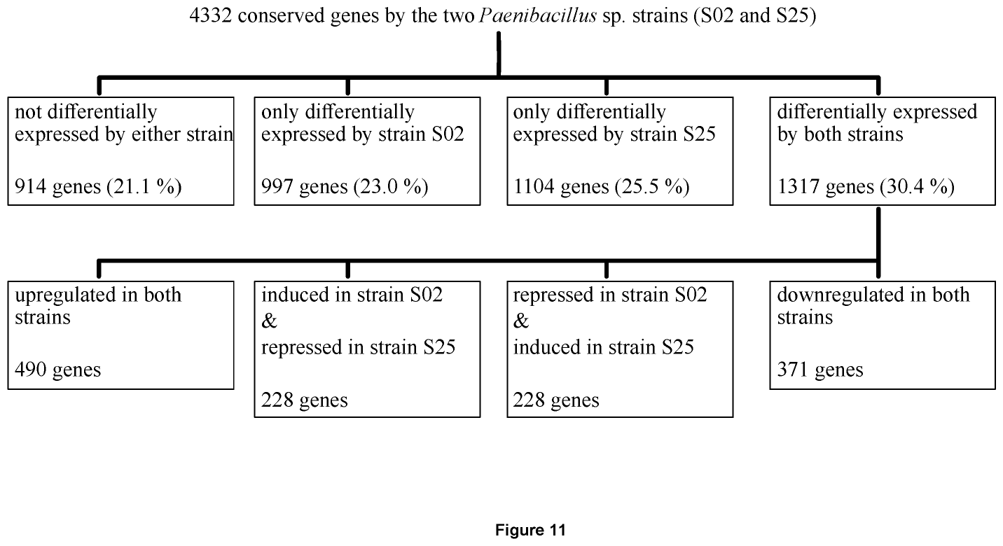

DGE analyses successfully identified genes that were differentially expressed caused by plant-bacteria interactions (Table 9). For bacteria, the DGE analyses compared transcriptome profiles of bacteria when barley seedlings were present and absent, and grown in two growth mediums. When NB was used, strain AR, S25 and S02 had 4009, 5013 and 5266 genes that passed the abundance filter respectively, and 1380, 2945, 2890 genes that were differentially expressed when seedlings were present. Moreover, Paenibacillus sp. strain S02 cultured in Burk's N-free medium had 5032 genes that passed the abundance filter and 2524 genes that were differentially expressed when seedlings were present. Interestingly, strain-specific responses were also identified using the two Paenibacillus sp. strains on NB (FIG. 11) despite the fact that the two strains are genetically similar (average nucleotide identity=97.78%) and share 4332 conserved genes (unpublished data). Among 4332 conserved genes, there were 997 genes that were only differentially expressed by strain S02 and 1104 genes that were only differentially expressed by strain S25. There were also 1317 genes that were differentially expressed by both strains, including 228 genes that were induced by strain S02 but represses by strain S25 and another 228 genes that were repressed by strain S02 but induced by strain S25. There were also 490 genes that were upregulated by both strains and 371 genes that were downregulated by both strains, and 914 genes that were not differentially expressed by either strain.

TABLE 4
Bacterial and plant genes that passed the abundance filter and
were differentially expressed identified by DGE analyses

| | | | | No. of genes | No. of |
|---|---|---|---|---|---|
| | | | | passed the | differentially |
| | | | | abundance | expressed |
| | Sample | Treatment | Medium | filter | genes |
| Bacteria | AR | Barley | NB | 4009 | 1380 |
| | S25 | seedling | | 5013 | 2945 |
| | S02 | | | 5266 | 2890 |
| | | | Burk's | 5032 | 2524 |
| | | | N-free | | |
| Plant | Barley | AR | NB | 37073 | 13948 |
| | seedling | S25 | | 35365 | 13648 |
| | | S02 | | 34798 | 9129 |
| | | | Burk's | 31502 | 10806 |
| | | | N-free | | |

AR: novel E. gerundensis strain
S02/S25: novel Paenibacillus sp. strains

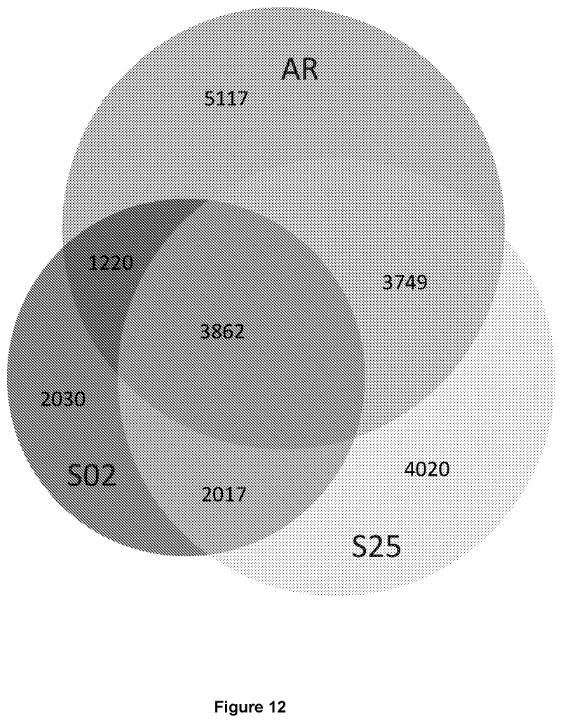

For barley, the DGE analyses compared transcriptome profiles in seedlings inoculated with AR, S25 and S02 (absence versus presence), and grown in different mediums. Barley seedlings co-incubated with bacterial strains AR, S25 and S02 in NB had 37073, 35365 and 34798 genes that passed the abundance filter respectively, and 13948, 13648 and 9129 genes that were differentially expressed when bacterial strains were present. When Burk's N-free medium was used, seedlings co-incubated with strain S02 had 31502 genes that passed the abundance filter and 10806 that were differentially expressed when the strain was present. Overall, 22015 barley genes were expressed differentially during the plant-bacteria interactions assay using NB, including 3862 genes that were shared by interactions with all three strains and 5117, 4020 and 2030 genes that were unique to interactions with strain AR, S25 and S02, respectively (FIG. 12). GO enrichment analysis using these 3862 shared genes identified an overrepresented (P<0.05) GO category associated with sequence-specific DNA binding (GO:0043565), which are commonly associated with transcriptional regulation. GO enrichment analysis of the 8067 differentially expressed barley genes that were associated with the two Paenibacillus sp. strains (S02 and S25) revealed overrepresented (P<0.05) GO categories associated with nitrogen metabolism, including nitrogen compound transport (GO:0015112) and organonitrogen compound metabolic process (GO:1901564). Moreover, compared with seedlings inoculated with Paenibacillus sp. strain S02, seedlings inoculated with Paenibacillus sp. strain S25 shared more differentially expressed genes with seedlings inoculated with E. gerundensis strain AR. GO enrichment analysis of the 7611 genes shared by seedling inoculated with strain S25 or AR revealed overrepresented (P<0.05) GO categories associated with stress responses (GO:0006950, GO:0006979). There were no overrepresented GO categories associated with disease responses and plant defence mechanisms detected using any differentially expressed barley genes.

Overall, novel Paenibacillus sp. strain S02 appeared like it formed a more congruent association with barley than novel Paenibacillus sp. strain S25 or E. gerundensis strain AR, as the transcriptional profile of uninoculated barley was very similar to novel strain S02 (FIG. 10). Furthermore, there were no overrepresented stress related GO categories in differentially expressed genes of novel strain S02 compared to novel strains S25 and AR, suggesting a more mutualistic interaction. The differentially expressed genes that had overrepresented GO categories were more associated with mutualism as they were related to nitrogen metabolism, which may be directly related to the nitrogen-fixation activity of strain S02 and the delivery of nitrogen to the host plant.

Reference to any prior art in the specification is not, and should not be taken as, an acknowledgment or any form of suggestion that this prior art forms part of the common general knowledge in Australia or any other jurisdiction or that this prior art could reasonably be expected to be combined by a person skilled in the art.

Finally, it is to be understood that various alterations, modifications and/or additions may be made without departing from the spirit of the present invention as outlined herein.

### REFERENCES

Baldani, J. I., Reis, V. M., Videira, S. S., Boddey, L. H., and Baldani, V. L. D. (2014). The art of isolating nitrogen-fixing bacteria from non-leguminous plants using N-free semi-solid media: a practical guide for microbiologists. Plant and Soil 384, 413-431.
Camacho, C., Coulouris, G., Avagyan, V., Ma, N., Papadopoulos, J., Bealer, K., and Madden, T. L. (2009). BLAST+: architecture and applications. BMC Bioinformatics 10, 421.
Chen, S., Zhou, Y., Chen, Y., and Gu, J. (2018). fastp: an ultra-fast all-in-one FASTQ preprocessor. Bioinformatics 34, i884-i890.
Choi, S. K., Park, S. Y., Kim, R., Kim, S. B., Lee, C. H., Kim, J. F., and Park, S. H. (2009). Identification of a polymyxin synthetase gene cluster of Paenibacillus polymyxa and heterologous expression of the gene in Bacillus subtilis. J Bacteriol 191, 3350-3358.
Chun, J., Oren, A., Ventosa, A., Christensen, H., Arahal, D. R., Da Costa, M. S., Rooney, A. P., Yi, H., Xu, X. W., De Meyer, S., and Trujillo, M. E. (2018). Proposed minimal standards for the use of genome data for the taxonomy of prokaryotes. Int J Syst Evol Microbiol 68, 461-466.
De Coster, W., D'hert, S., Schultz, D. T., Cruts, M., and Van Broeckhoven, C. (2018). NanoPack: visualizing and processing long read sequencing data. Bioinformatics.
De Werra, P., Pechy-Tarr, M., Keel, C., and Maurhofer, M. (2009). Role of gluconic acid production in the regulation of biocontrol traits of Pseudomonas fluorescens CHA0. Appl Environ Microbiol 75, 4162-4174.
Eastman, A. W., Heinrichs, D. E., and Yuan, Z.-C. (2014). Comparative and genetic analysis of the four sequenced Paenibacillus polymyxa genomes reveals a diverse metabolism and conservation of genes relevant to plant-growth promotion and competitiveness. BMC Genomics 15, 851.
Franche, C., LindstrOm, K., and Elmerich, C. (2009). Nitrogen-fixing bacteria associated with leguminous and non-leguminous plants. Plant and soil 321, 35-59.
Li, T., Mann, R., Sawbridge, T., Kaur, J., Auer, D., and Spangenberg, G. (2020). Novel Xanthomonas Species From the Perennial Ryegrass Seed Microbiome—Assessing the Bioprotection Activity of Non-pathogenic Relatives of Pathogens. Frontiers in Microbiology 11, 1991.
Li, J., and Jensen, S. E. (2008). Nonribosomal biosynthesis of fusaricidins by Paenibacillus polymyxa PKB1 involves direct activation of a D-amino acid. Chem Biol 15, 118-127.
Li, Y., and Chen S. (2019) Fusaricidin Produced by Paenibacillus polymyxa WLY78 Induces Systemic Resistance against Fusarium Wilt of Cucumber. Int. J. Mol. Sci 20(20):5240.
Liu, H., Liu, K., Li, Y., Wang, C., Hou, Q., Xu, W., Fan, L., Zhao, J., Gou, J., Du, B., and Ding, Y. (2017). Complete Genome Sequence of Paenibacillus polymyxa YC0136, a Plant Growth—Promoting Rhizobacterium Isolated from Tobacco Rhizosphere. Genome Announcements 5, e01635-01616.
Lohans, C. T., Van Belkum, M. J., Cochrane, S. A., Huang, Z., Sit, C. S., Mcmullen, L. M., and Vederas, J. C. (2014). Biochemical, structural, and genetic characterization of tridecaptin A₁, an antagonist of Campylobacter jejuni. Chembiochem 15, 243-249.
Lugtenberg, B., and Kamilova, F. (2009). Plant-growth-promoting rhizobacteria. Annual review of microbiology 63, 541-556.
Page, A. J., Cummins, C. A., Hunt, M., Wong, V. K., Reuter, S., Holden, M. T., Fookes, M., Falush, D., Keane, J. A., and Parkhill, J. (2015). Roary: rapid large-scale prokaryote pan genome analysis. Bioinformatics 31, 3691-3693.
Park, J. E., Kim, H. R., Park, S. Y., Choi, S. K., and Park, S. H. (2017). Identification of the biosynthesis gene cluster for the novel lantibiotic paenilan from Paenibacillus polymyxa E681 and characterization of its product. J Appl Microbiol 123, 1133-1147.
Patro, R., Duggal, G., Love, M. I., Irizarry, R. A., and Kingsford, C. (2017). Salmon provides fast and bias-aware quantification of transcript expression. Nat Methods 14, 417-419.
Pimentel, H., Bray, N. L., Puente, S., Melsted, P., and Pachter, L. (2017). Differential analysis of RNA-seq incorporating quantification uncertainty. Nat Methods 14, 687-690.
Price, M. N., Dehal, P. S., and Arkin, A. P. (2010). FastTree 2—approximately maximum-likelihood trees for large alignments. PLoS One 5, e9490.
Rapazote-Flores, P., Bayer, M., Milne, L., Mayer, C. D., Fuller, J., Guo, W., Hedley, P. E., Morris, J., Halpin, C., Kam, J., Mckim, S. M., Zwirek, M., Casao, M. C., Barakate, A., Schreiber, M., Stephen, G., Zhang, R., Brown, J. W. S., Waugh, R., and Simpson, C. G. (2019). BaRTv1.0: an improved barley reference transcript dataset to determine accurate changes in the barley transcriptome using RNA-seq. BMC Genomics 20, 968.
Reeve, W., O'hara, G., Chain, P., Ardley, J., Brau, L., Nandesena, K., Tiwari, R., Copeland, A., Nolan, M., Han, C., Brettin, T., Land, M., Ovchinikova, G., Ivanova, N., Mavromatis, K., Markowitz, V., Kyrpides, N., Melino, V., Denton, M., Yates, R., and Howieson, J. (2010). Complete genome sequence of Rhizobium leguminosarum bv. trifolii strain WSM1325, an effective microsymbiont of annual Mediterranean clovers. Stand Genomic Sci 2, 347-356.
Richter, M., and Rosselló-Móra, R. (2009). Shifting the genomic gold standard for the prokaryotic species definition. Proceedings of the National Academy of Sciences 106, 19126-19131.
Tannenbaum, I., Kaur, J., Mann, R., Sawbridge, T., Rodoni, B., and Spangenberg, G. (2020). Profiling the Lolium perenne Microbiome: From Seed to Seed. Phytobiomes Journal 4, 281-289.
Vater, J., Herfort, S., Doellinger, J., Weydmann, M., Borriss, R., and Lasch, P. (2018). Genome Mining of the Lipopeptide Biosynthesis of Paenibacillus polymyxa E681 in Combination with Mass Spectrometry: Discovery of the Lipoheptapeptide Paenilipoheptin. Chembiochem 19, 744-753.
Weber, T., Blin, K., Duddela, S., Krug, D., Kim, H. U., Bruccoleri, R., Lee, S. Y., Fischbach, M. A., Muller, R., Wohlleben, W., Breitling, R., Takano, E., and Medema, M. H. (2015). antiSMASH 3.0—a comprehensive resource for the genome mining of biosynthetic gene clusters. Nucleic Acids Res 43, W237-243.
Wick, R. R., Judd, L. M., Gorrie, C. L., and Holt, K. E. (2017). Unicycler: Resolving bacterial genome assemblies from short and long sequencing reads. PLOS Computational Biology 13, e1005595.
Wick, R. R., Schultz, M. B., Zobel, J., and Holt, K. E. (2015). Bandage: interactive visualization of de novo genome assemblies. Bioinformatics 31, 3350-3352.
Wood, D. E., Lu, J. & Langmead, B. (2019). Improved metagenomic analysis with Kraken 2. Genome Biol 20, 257
Wood, D. E., and Salzberg, S. L. (2014). Kraken: ultrafast metagenomic sequence classification using exact alignments. Genome Biology 15, R46.
Xie, J.-B., Du, Z., Bai, L., Tian, C., Zhang, Y., Xie, J.-Y., Wang, T., Liu, X., Chen, X., Cheng, Q., Chen, S., and Li, J. (2014). Comparative Genomic Analysis of N2-Fixing and Non-N2-Fixing Paenibacillus spp.: Organization, Evolution and Expression of the Nitrogen Fixation Genes. PLOS Genetics 10, e1004231.
Xie, J., Shi, H., Du, Z., Wang, T., Liu, X., and Chen, S. (2016). Comparative genomic and functional analysis reveal conservation of plant growth promoting traits in Paenibacillus polymyxa and its closely related species. Sci Rep 6, 21329.
Yuan, Z.-C., Zaheer, R., and Finan, T. M. (2006). Regulation and Properties of PstSCAB, a High-Affinity, High-Velocity Phosphate Transport System of Sinorhizobium meliloti. Journal of Bacteriology 188, 1089-1102. Spaepen et al., 2007
Zhu, S., Hegemann, J. D., Fage, C. D., Zimmermann, M., Xie, X., Linne, U., & Marahiel, M. A. (2016). Insights into the Unique Phosphorylation of the Lasso Peptide Paeninodin. The Journal of biological chemistry, 291(26), 13662-13678.

1-29. (canceled)

30. A substantially purified or isolated endophyte strain isolated from a plant of the Poaceae family, wherein said endophyte is a strain of Paenibacillus sp. which provides bioprotection and/or biofertilizer phenotypes to plants into which it is inoculated.

31. An endophyte according to claim 30, wherein the bioprotection and/or biofertilizer phenotype includes production of the bioprotectant compound in the plant into which the endophyte is inoculated.

32. An endophyte according to claim 30, wherein the bioprotection and/or biofertilizer phenotype is selected from the group consisting of:
nitrogen fixation;
phosphate solubilisation and/or assimilation;
production of organic acids; and
production of secondary metabolites
in the plant into which the endophyte is inoculated.

33. An endophyte according to claim 30, wherein the endophyte is selected from:
i) strain S02 as described herein and as deposited with The National Measurement Institute of 1/153 Bertie St, Port Melbourne, Victoria 3207 Australia on 19 Feb. 2021 with accession number V21/003092; or
ii) strain S25 as described herein and as deposited with The National Measurement Institute of 1/153 Bertie St, Port Melbourne, Victoria 3207 Australia on 19 Feb. 2021 with accession number V21/003093.

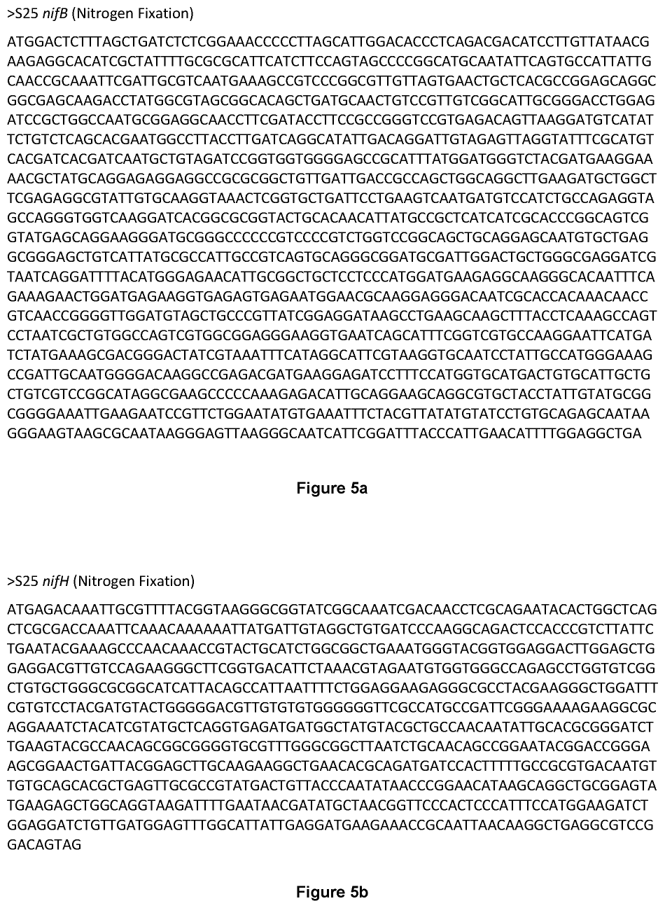

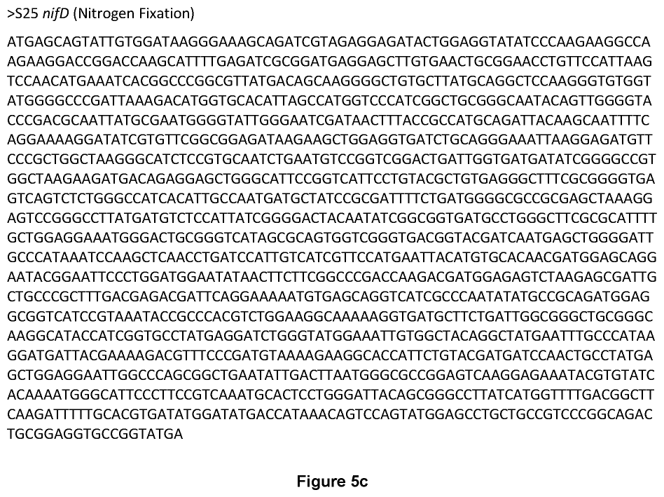

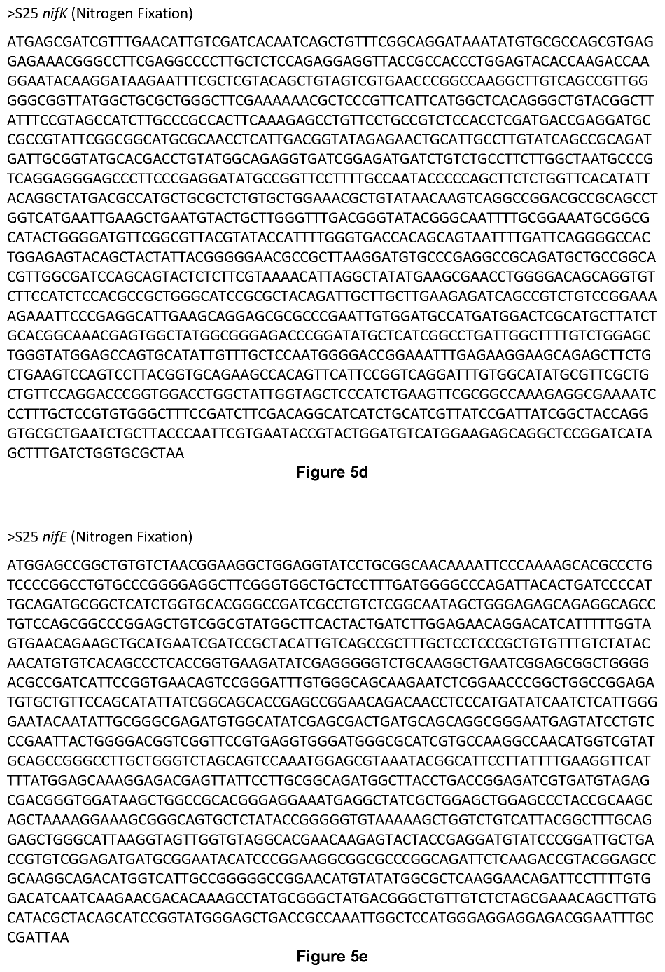

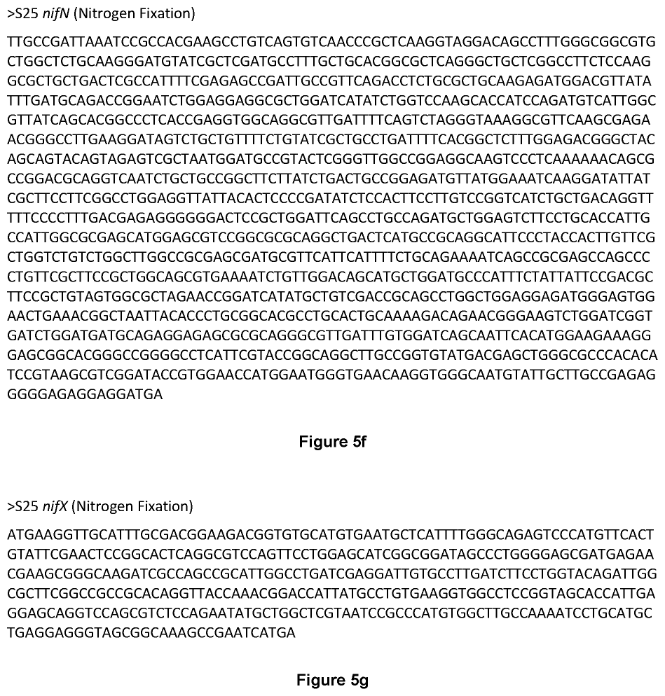

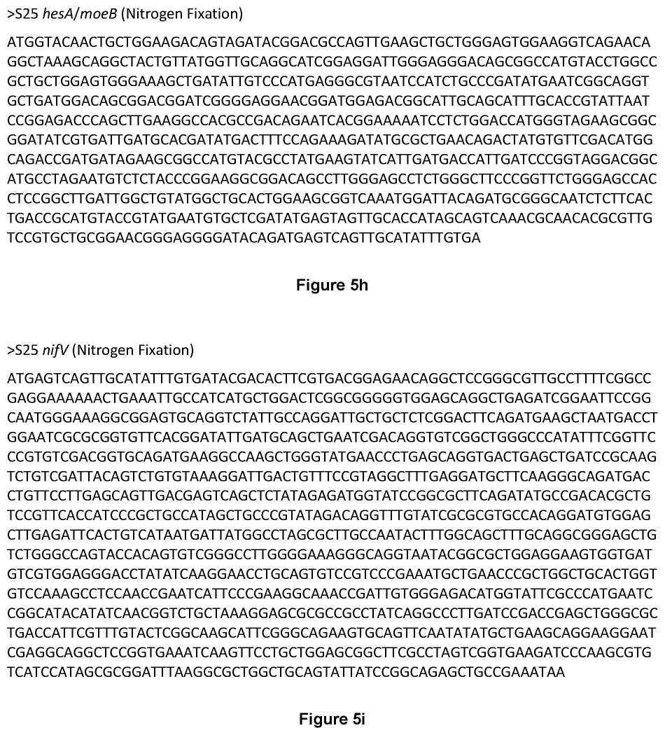

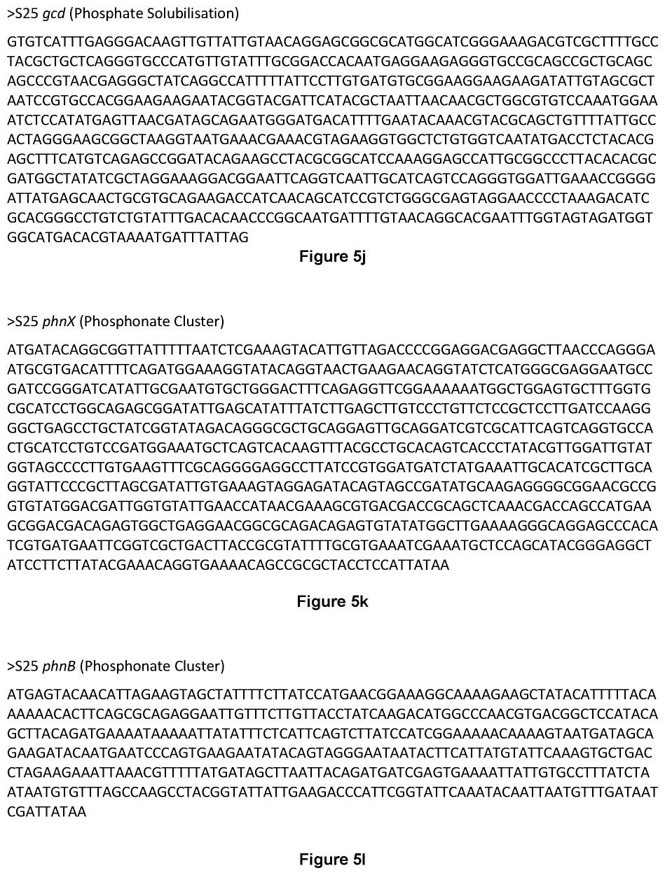

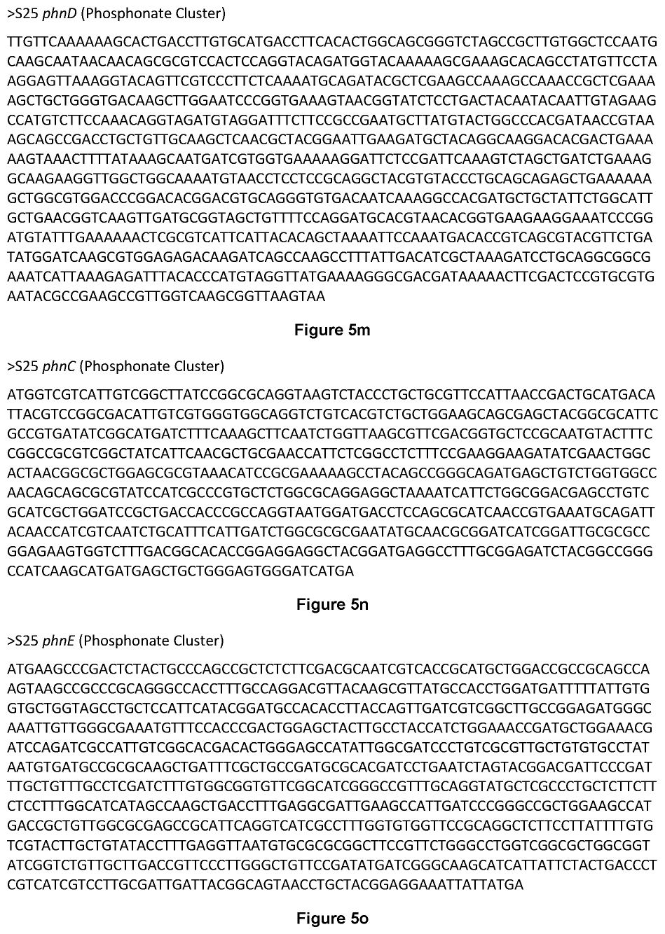

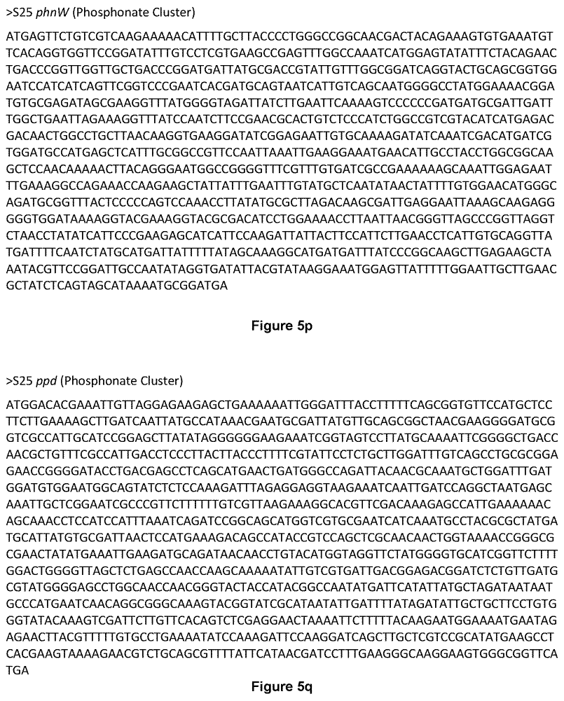

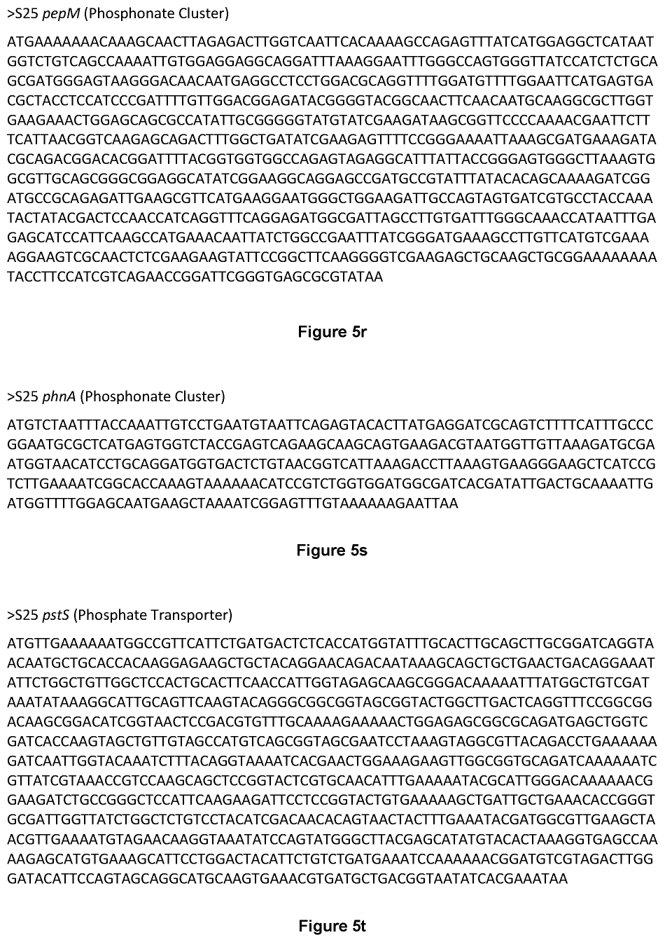

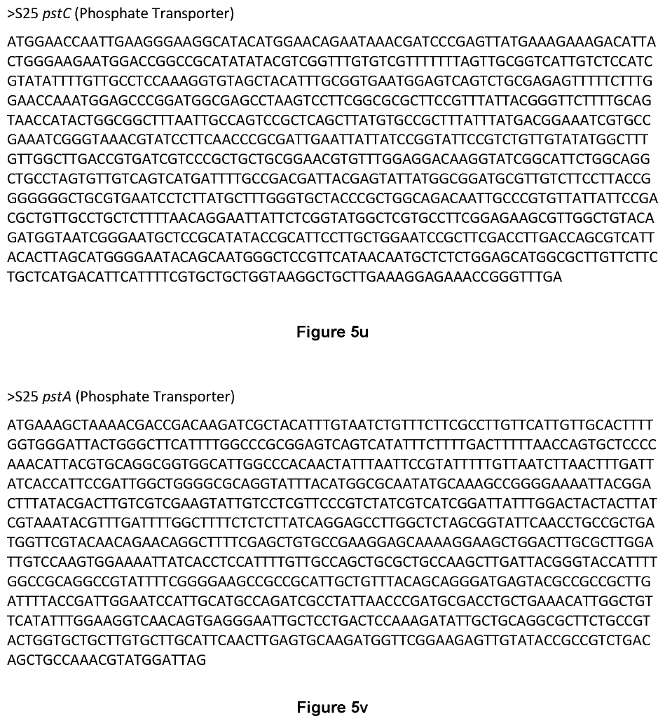

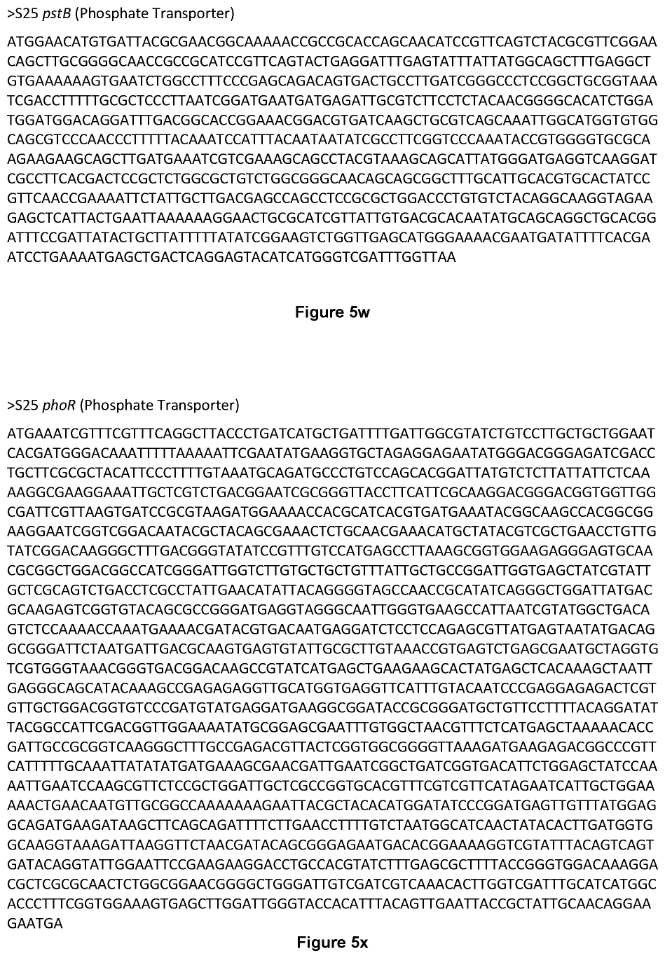

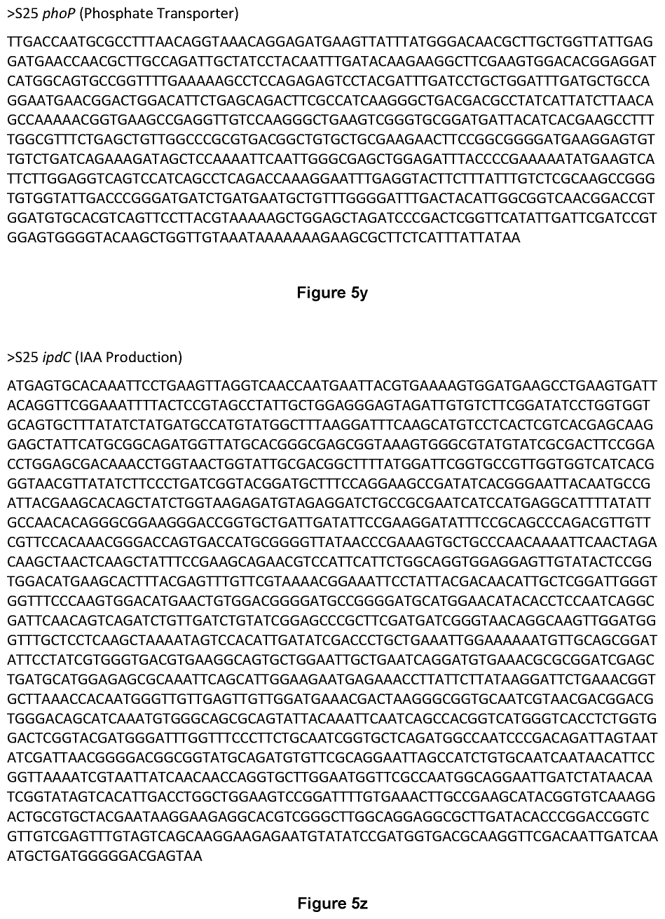

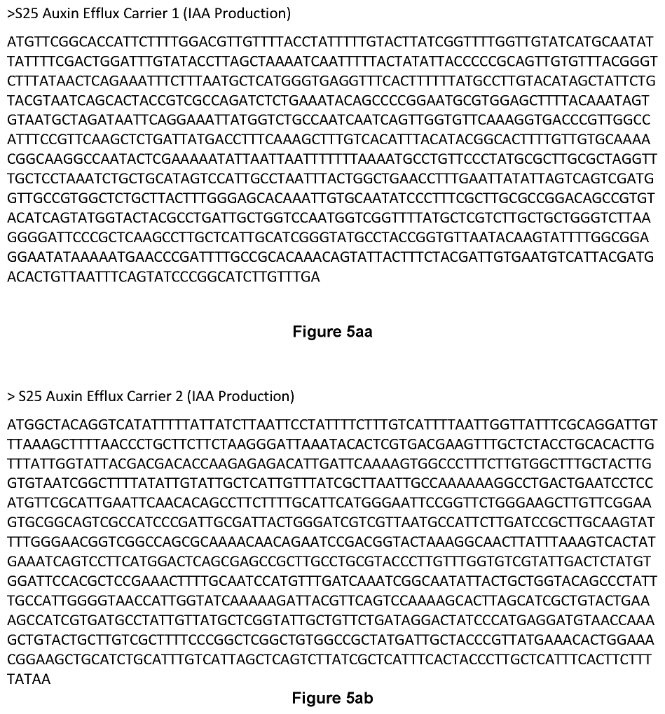

34. An endophyte according to claim 30, wherein the bioprotection and/or biofertilizer phenotype is a result of the differential gene expression of one or more gene(s) selected from
i) nitrogen fixation genes as shown in FIGS. 5a to 5ac (SEQ ID NOS: 3 to 31, respectively) or FIGS. 6a to 6ac (SEQ ID NOS: 32 to 60, respectively);
ii) phosphate solubilisation, phosphonate cluster (phn) and/or phosphate transporter (pst) genes as shown in FIGS. 5a to 5ac (SEQ ID NOS: 3 to 31, respectively) or FIGS. 6a to 6ac (SEQ ID NOS: 32 to 60, respectively); or
iii) Indole-3-acetic acid (IAA) production genes as shown in FIGS. 5a to 5ac (SEQ ID NOS: 3 to 31, respectively) or FIGS. 6a to 6ac (SEQ ID NOS: 32 to 60, respectively).

35. An endophyte according to claim 30, wherein bioprotection and/or biofertilizer phenotype includes one or both of:
i) resistance to harmful fungal pathogen growth; and
ii) production of secondary metabolites with antimicrobial bioactivity.

36. An endophyte according to claim 35, wherein the harmful fungal pathogen is selected from Fusarium spp., Verticillium albo-atrum, Monilia persoon, Alternaria mali, Botrytis cinerea, and Aspergillus niger.

37. An endophyte according to claim 35, wherein the harmful fungal pathogen is selected from Colletotrichum graminicola and Fusarium verticillioides.

38. An endophyte according to claim 35, wherein the secondary metabolite is a result of differential gene expression of one or more gene cluster(s) as shown in FIG. 9 (SEQ ID NOS: 61 to 66, respectively).

39. A plant or part thereof infected with one or more endophytes according to claim 30.

40. A plant, plant seed or other plant part derived from a plant or part thereof according to claim 39 and stably infected with said one or more endophytes.

41. A method for producing a bioprotectant compound said method including infecting a plant with an endophyte according to claim 30 and cultivating the plant under conditions suitable to produce the bioprotectant compound and optionally isolating the bioprotectant compound from the plant.

42. A method for producing a bioprotectant compound, said method including culturing an endophyte according to claim 30 under conditions suitable to produce the bioprotectant compound and optionally isolating the bioprotectant compound from the culture medium.

43. A method of increasing nitrogen use efficiency or increasing nitrogen fixation by a plant, said method including infecting a plant with a bioprotectant compound producing endophyte according to claim 30, and cultivating the plant.

44. A method according to claim 43, wherein one or both of the following applies:
i) the plant is cultivated in the presence of soil nitrogen and/or applied nitrogen; and
ii) the plant is cultivated in soil.

45. The plant, plant seed or plant part thereof according to claim 39 wherein the plant into which the endophyte is inoculated is an endophyte-free host plant or part thereof before being stably infected with said endophyte.

46. The plant, plant seed or plant part thereof according to claim 39, wherein the plant into which the endophyte is inoculated is an agricultural plant species selected from one or more of forage grass, turf grass, bioenergy grass, grain crop and industrial crop.

47. An endophyte according claim 46, wherein the plant into which the endophyte is inoculated is selected from one of the groups consisting of:
i) a forage, turf or bioenergy grass selected from the group consisting of those belonging to the genera Lolium and Festuca, including L. perenne (perennial ryegrass), L. arundinaceum (tall fescue) and L. multiflorum (Italian ryegrass), and those belonging to the Brachiaria-Urochloa species complex (panic grasses), including Brachiaria brizantha, Brachiaria decumbens, Brachiaria humidicola, Brachiaria stolonifera, Brachiaria ruziziensis, B. dictyoneura, Urochloa brizantha, Urochloa decumbens, Urochloa humidicola, Urochloa mosambicensis as well as interspecific and intraspecific hybrids of Brachiaria-Urochloa species complex such as interspecific hybrids between Brachiaria ruziziensis x Brachiaria brizantha, Brachiaria ruziziensis x Brachiaria decumbens, [Brachiaria ruziziensis x Brachiaria decumbens] x Brachiaria brizantha, [Brachiaria ruziziensis x Brachiaria brizantha] x Brachiaria decumbens;
ii) those belonging to the genus Triticum, including T aestivum (wheat), those belonging to the genus Hordeum, including H. vulgare (barley), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), and those belonging to the genera Miscanthus, Paspalum, Pennisetum, Poa, Eragrostis and Agrostis; and
iii) a grain crop or industrial crop selected from the group consisting or wheat, barley, oats, chickpeas, triticale, fava beans, lupins, field peas, canola, cereal rye, vetch, lentils, millet/panicum, safflower, linseed, sorghum, sunflower, maize, canola, mungbeans, soybeans and cotton.


SEQUENCE LISTING (66 sequences)

SEQ ID NO: 1 - DNA, 1554 bp, Paenibacillus sp.
aatggagagtttgatcctggctcaggacgaacgctggcggcgtgcctaatacatgcaagtcgagcggggttatgtagaagcttgcttctacataacctagcggcggacgggtgagtaacacgtaggcaacctgcccacaagacagggataactaccggaaacggtagctaatacccgatacatccttttcctgcatgggagaaggaggaaagacggagcaatctgtcacttgtggatgggcctgcggcgcattagctagttggtggggtaaaggcctaccaaggcgacgatgcgtagccgacctgagagggtgatcggccacactgggactgagacacggcccagactcctacgggaggcagcagtagggaatcttccgcaatgggcgaaagcctgacggagcaacgccgcgtgagtgatgaaggttttcggatcgtaaagctctgttgccagggaagaacgtcttgtagagtaactgctacaagagtgacggtacctgagaagaaagccccggctaactacgtgccagcagccgcggtaatacgtagggggcaagcgttgtccggaattattgggcgtaaagcgcgcgcaggcggctctttaagtctggtgtttaatcccgaggctcaacttcgggtcgcactggaaactggggagcttgagtgcagaagaggagagtggaattccacgtgtagcggtgaaatgcgtagatatgtggaggaacaccagtggcgaaggcgactctctgggctgtaactgacgctgaggcgcgaaagcgtggggagcaaacaggattagataccctggtagtccacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgatcggtctagagataggcctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaaatcgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtggggtaacccgcaagggagccagccgccgaaggtggggtagatgattggggtgaagtcgtaacaaggtagccgtatcggaaggtgcggctggatcacctccttt

SEQ ID NO: 2 - DNA, 1554 bp, Paenibacillus sp.
aatggagagtttgatcctggctcaggacgaacgctggcggcgtgcctaatacatgcaagtcgagcggggttaattagaagcttgcttctaattaacctagcggcggacgggtgagtaacacgtaggcaacctgcccacaagacagggataactaccggaaacggtagctaatacccgatacatccttttcctgcatgggagaaggaggaaagacggagcaatctgtcacttgtggatgggcctgcggcgcattagctagttggtggggtaaaggcctaccaaggcgacgatgcgtagccgacctgagagggtgatcggccacactgggactgagacacggcccagactcctacgggaggcagcagtagggaatcttccgcaatgggcgaaagcctgacggagcaacgccgcgtgagtgatgaaggttttcggatcgtaaagctctgttgccagggaagaacgtcttgtagagtaactgctacaagagtgacggtacctgagaagaaagccccggctaactacgtgccagcagccgcggtaatacgtagggggcaagcgttgtccggaattattgggcgtaaagcgcgcgcaggcggctctttaagtctggtgtttaatcccgaggctcaacttcgggtcgcactggaaactggggagcttgagtgcagaagaggagagtggaattccacgtgtagcggtgaaatgcgtagatatgtggaggaacaccagtggcgaaggcgactctctgggctgtaactgacgctgaggcgcgaaagcgtggggagcaaacaggattagataccctggtagtccacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatagacctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaaatcgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtggggtaacccgcaagggagccagccgccgaaggtggggtagatgattggggtgaagtcgtaacaaggtagccgtatcggaaggtgcggctggatcacctccttt

SEQ ID NO: 3 - DNA, 1500 bp, Paenibacillus sp.
atggactctttagctgatctctcggaaacccccttagcattggacaccctcagacgacatccttgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagacctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatattgacaggattgtagagttaggtatttcgcatgtcacgatcacgatcaatgctgtagatccggtggtggggagccgcatttatggatgggtctacgatgaaggaaaacgctatgcaggagaggaggccgcgcggctgttgattgaccgccagctggcaggcttgaagatgctggcttcgagaggcgtattgtgcaaggtaaactcggtgctgattcctgaagtcaatgatgtccatctgccagaggtagccagggtggtcaaggatcacggcgcggtactgcacaacattatgccgctcatcatcgcacccggcagtcggtatgagcaggaagggatgcgggccccccgtccccgtctggtccggcagctgcaggagcaatgtgctgaggcgggagctgtcattatgcgccattgccgtcagtgcagggcggatgcgattggactgctgggcgaggatcgtaatcaggattttacatgggagaacattgcggctgctcctcccatggatgaagaggcaagggcacaatttcagaaagaactggatgagaaggtgagagtgagaatggaacgcaaggagggacaatcgcaccacaaacaaccgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctattgccatgggaaagccgattgcaatggggacaaggccgagacgatgaaggagatcctttccatggtgcatgactgtgcattgctgctgtcgtccggcataggcgaagcccccaaagagacattgcaggaagcaggcgtgctacctattgtatgcggcggggaaattgaagaatccgttctggaatatgtgaaatttctacgttatatgtatcctgtgcagagcaataagggaagtaagcgcaataagggagttaagggcaatcattcggatttacccattgaacattttggaggctga

SEQ ID NO: 4 - DNA, 867 bp, Paenibacillus sp.
atgagacaaattgcgttttacggtaagggcggtatcggcaaatcgacaacctcgcagaatacactggctcagctcgcgaccaaattcaaacaaaaaattatgattgtaggctgtgatcccaaggcagactccacccgtcttattctgaatacgaaagcccaacaaaccgtactgcatctggcggctgaaatgggtacggtggaggacttggagctggaggacgttgtccagaagggcttcggtgacattctaaacgtagaatgtggtgggccagagcctggtgtcggctgtgctgggcgcggcatcattacagccattaattttctggaggaagagggcgcctacgaagggctggatttcgtgtcctacgatgtactgggggacgttgtgtgtggggggttcgccatgccgattcgggaaaagaaggcgcaggaaatctacatcgtatgctcaggtgagatgatggctatgtacgctgccaacaatattgcacgcgggatcttgaagtacgccaacagcggcggggtgcgtttgggcggcttaatctgcaacagccggaatacggaccgggaagcggaactgattacggagcttgcaagaaggctgaacacgcagatgatccactttttgccgcgtgacaatgttgtgcagcacgctgagttgcgccgtatgactgttacccaatataacccggaacataagcaggctgcggagtatgaagagctggcaggtaagattttgaataacgatatgctaacggttcccactcccatttccatggaagatctggaggatctgttgatggagtttggcattattgaggatgaagaaaccgcaattaacaaggctgaggcgtccggacagtag

SEQ ID NO: 5 - DNA, 1449 bp, Paenibacillus sp.
atgagcagtattgtggataagggaaagcagatcgtagaggagatactggaggtatatcccaagaaggccaagaaggaccggaccaagcattttgagatcgcggatgaggagcttgtgaactgcggaacctgttccattaagtccaacatgaaatcacggcccggcgttatgacagcaaggggctgtgcttatgcaggctccaagggtgtggtatggggcccgattaaagacatggtgcacattagccatggtcccatcggctgcgggcaatacagttggggtacccgacgcaattatgcgaatggggtattgggaatcgataactttaccgccatgcagattacaagcaattttcaggaaaaggatatcgtgttcggcggagataagaagctggaggtgatctgcagggaaattaaggagatgttcccgctggctaagggcatctccgtgcaatctgaatgtccggtcggactgattggtgatgatatcggggccgtggctaagaagatgacagaggagctgggcattccggtcattcctgtacgctgtgagggctttcgcggggtgagtcagtctctgggccatcacattgccaatgatgctatccgcgattttctgatggggcgccgcgagctaaaggagtccgggccttatgatgtctccattatcggggactacaatatcggcggtgatgcctgggcttcgcgcattttgctggaggaaatgggactgcgggtcatagcgcagtggtcgggtgacggtacgatcaatgagctggggattgcccataaatccaagctcaacctgatccattgtcatcgttccatgaattacatgtgcacaacgatggagcaggaatacggaattccctggatggaatataacttcttcggcccgaccaagacgatggagagtctaagagcgattgctgcccgctttgacgagacgattcaggaaaaatgtgagcaggtcatcgcccaatatatgccgcagatggaggcggtcatccgtaaataccgcccacgtctggaaggcaaaaaggtgatgcttctgattggcgggctgcgggcaaggcataccatcggtgcctatgaggatctgggtatggaaattgtggctacaggctatgaatttgcccataaggatgattacgaaaagacgtttcccgatgtaaaagaaggcaccattctgtacgatgatccaactgcctatgagctggaggaattggcccagcggctgaatattgacttaatgggcgccggagtcaaggagaaatacgtgtatcacaaaatgggcattcccttccgtcaaatgcactcctgggattacagcgggccttatcatggttttgacggcttcaagatttttgcacgtgatatggatatgaccataaacagtccagtatggagcctgctgccgtcccggcagactgcggaggtgccggtatga

SEQ ID NO: 6 - DNA, 1530 bp, Paenibacillus sp.
atgagcgatcgtttgaacattgtcgatcacaatcagctgtttcggcaggataaatatgtgcgccagcgtgaggagaaacgggccttcgaggccccttgctctccagaggaggttaccgccaccctggagtacaccaagaccaaggaatacaaggataagaatttcgctcgtacagctgtagtcgtgaacccggccaaggcttgtcagccgttgggggcggttatggctgcgctgggcttcgaaaaaacgctcccgttcattcatggctcacagggctgtacggcttatttccgtagccatcttgcccgccacttcaaagagcctgttcctgccgtctccacctcgatgaccgaggatgccgccgtattcggcggcatgcgcaacctcattgacggtatagagaactgcattgccttgtatcagccgcagatgattgcggtatgcacgacctgtatggcagaggtgatcggagatgatctgtctgccttcttggctaatgcccgtcaggagggagcccttcccgaggatatgccggttccttttgccaatacccccagcttctctggttcacatattacaggctatgacgccatgctgcgctctgtgctggaaacgctgtataacaagtcaggccggacgccgcagcctggtcatgaattgaagctgaatgtactgcttgggtttgacgggtatacgggcaattttgcggaaatgcggcgcatactggggatgttcggcgttacgtataccattttgggtgaccacagcagtaattttgattcaggggccactggagagtacagctactattacgggggaacgccgcttaaggatgtgcccgaggccgcagatgctgccggcacgttggcgatccagcagtactctcttcgtaaaacattaggctatatgaagcgaacctggggacagcaggtgtcttccatctccacgccgctgggcatccgcgctacagattgcttgcttgaagagatcagccgtctgtccggaaaagaaattcccgaggcattgaagcaggagcgcgcccgaattgtggatgccatgatggactcgcatgcttatctgcacggcaaacgagtggctatggcgggagacccggatatgctcatcggcctgattggcttttgtctggagctgggtatggagccagtgcatattgtttgctccaatggggaccggaaatttgagaaggaagcagagcttctgctgaagtccagtccttacggtgcagaagccacagttcattccggtcaggatttgtggcatatgcgttcgctgctgttccaggacccggtggacctggctattggtagctcccatctgaagttcgcggccaaagaggcgaaaatccctttgctccgtgtgggctttccgatcttcgacaggcatcatctgcatcgttatccgattatcggctaccagggtgcgctgaatctgcttacccaattcgtgaataccgtactggatgtcatggaagagcaggctccggatcatagctttgatctggtgcgctaa

SEQ ID NO: 7 - DNA, 1362 bp, Paenibacillus sp.
atggagccggctgtgtctaacggaaggctggaggtatcctgcggcaacaaaattcccaaaagcacgccctgtccccggcctgtgcccggggaggcttcgggtggctgctcctttgatggggcccagattacactgatccccattgcagatgcggctcatctggtgcacgggccgatcgcctgtctcggcaatagctgggagagcagaggcagcctgtccagcggcccggagctgtcggcgtatggcttcactactgatcttggagaacaggacatcatttttggtagtgaacagaagctgcatgaatcgatccgctacattgtcagccgctttgctcctcccgctgtgtttgtctatacaacatgtgtcacagccctcaccggtgaagatatcgagggggtctgcaaggctgaatcggagcggctggggacgccgatcattccggtgaacagtccgggatttgtgggcagcaagaatctcggaacccggctggccggagatgtgctgttccagcatattatcggcagcaccgagccggaacagacaacctcccatgatatcaatctcattggggaatacaatattgcgggcgagatgtggcatatcgagcgactgatgcagcaggcgggaatgagtatcctgtcccgaattactggggacggtcggttccgtgaggtgggatgggcgcatcgtgccaaggccaacatggtcgtatgcagccgggccttgctgggtctagcagtccaaatggagcgtaaatacggcattccttattttgaaggttcattttatggagcaaaggagacgagttattccttgcggcagatggcttacctgaccggagatcgtgatgtagagcgacgggtggataagctggccgcacgggaggaaatgaggctatcgctggagctggagccctaccgcaagcagctaaaaggaaagcgggcagtgctctataccgggggtgtaaaaagctggtctgtcattacggctttgcaggagctgggcattaaggtagttggtgtaggcacgaacaagagtactaccgaggatgtatcccggattgctgaccgtgtcggagatgatgcggaatacatcccggaaggcggcgcccggcagattctcaagaccgtacggagccgcaaggcagacatggtcattgccgggggccggaacatgtatatggcgctcaaggaacagattccttttgtggacatcaatcaagaacgacacaaagcctatgcgggctatgacgggctgttgtctctagcgaaacagcttgtgcatacgctacagcatccggtatgggagctgaccgccaaattggctccatgggaggaggagacggaatttgccgattaa

SEQ ID NO: 8 - DNA, 1308 bp, Paenibacillus sp.
ttgccgattaaatccgccacgaagcctgtcagtgtcaacccgctcaaggtaggacagcctttgggcggcgtgctggctctgcaagggatgtatcgctcgatgcctttgctgcacggcgctcagggctgctcggccttctccaaggcgctgctgactcgccattttcgagagccgattgccgttcagacctctgcgctgcaagagatggacgttatatttgatgcagaccggaatctggaggaggcgctggatcatatctggtccaagcaccatccagatgtcattggcgttatcagcacggccctcaccgaggtggcaggcgttgattttcagtctagggtaaaggcgttcaagcgagaacgggccttgaaggatagtctgctgttttctgtatcgctgcctgattttcacggctctttggagacgggctacagcagtacagtagagtcgctaatggatgccgtactcgggttggccggaggcaagtccctcaaaaaacagcgccggacgcaggtcaatctgctgccggcttcttatctgactgccggagatgttatggaaatcaaggatattatcgcttccttcggcctggaggttattacactccccgatatctccacttccttgtccggtcatctgctgacaggtttttcccctttgacgagaggggggactccgctggattcagcctgccagatgctggagtcttcctgcaccattgccattggcgcgagcatggagcgtccggcgcgcaggctgactcatgccgcaggcattccctaccacttgttcgctggtctgtctggcttggccgcgagcgatgcgttcattcattttctgcagaaaatcagccgcgagccagcccctgttcgcttccgctggcagcgtgaaaatctgttggacagcatgctggatgcccatttctattattccgacgcttccgctgtagtggcgctagaaccggatcatatgctgtcgaccgcagcctggctggaggagatgggagtggaactgaaacggctaattacaccctgcggcacgcctgcactgcaaaagacagaacgggaagtctggatcggtgatctggatgatgcagaggagagcgcgcagggcgttgatttgtggatcagcaattcacatggaagaaagggagcggcacgggccggggcctcattcgtaccggcaggcttgccggtgtatgacgagctgggcgcccacacatccgtaagcgtcggataccgtggaaccatggaatgggtgaacaaggtgggcaatgtattgcttgccgagagggggagaggaggatga

SEQ ID NO: 9 - DNA, 390 bp, Paenibacillus sp.
atgaaggttgcatttgcgacggaagacggtgtgcatgtgaatgctcattttgggcagagtcccatgttcactgtattcgaactccggcactcaggcgtccagttcctggagcatcggcggatagccctggggagcgatgagaacgaagcgggcaagatcgccagccgcattggcctgatcgaggattgtgccttgatcttcctggtacagattggcgcttcggccgccgcacaggttaccaaacggaccattatgcctgtgaaggtggcctccggtagcaccattgaggagcaggtccagcgtctccagaatatgctggctcgtaatccgcccatgtggcttgccaaaatcctgcatgctgaggagggtagcggcaaagccgaatcatga

SEQ ID NO: 10 - DNA, 765 bp, Paenibacillus sp.
atggtacaactgctggaagacagtagatacggacgccagttgaagctgctgggagtggaaggtcagaacaggctaaagcaggctactgttatggttgcaggcatcggaggattgggagggacagcggccatgtacctggccgctgctggagtgggaaagctgatattgtcccatgagggcgtaatccatctgcccgatatgaatcggcaggtgctgatggacagcggacggatcggggaggaacggatggagacggcattgcagcatttgcaccgtattaatccggagacccagcttgaaggccacgccgacagaatcacggaaaaatcctctggaccatgggtagaagcggcggatatcgtgattgatgcacgatatgactttccagaaagatatgcgctgaacagactatgtgttcgacatggcagaccgatgatagaagcggccatgtacgcctatgaagtatcattgatgaccattgatcccggtaggacggcatgcctagaatgtctctacccggaaggcggacagccttgggagcctctgggcttcccggttctgggagccacctccggcttgattggctgtatggctgcactggaagcggtcaaatggattacagatgcgggcaatctcttcactgaccgcatgtaccgtatgaatgtgctcgatatgagtagttgcaccatagcagtcaaacgcaacacgcgttgtccgtgctgcggaacgggaggggatacagatgagtcagttgcatatttgtga

SEQ ID NO: 11 - DNA, 1137 bp, Paenibacillus sp.
atgagtcagttgcatatttgtgatacgacacttcgtgacggagaacaggctccgggcgttgccttttcggccgaggaaaaaactgaaattgccatcatgctggactcggcgggggtggagcaggctgagatcggaattccggcaatgggaaaggcggagtgcaggtctattgccaggattgctgctctcggacttcagatgaagctaatgacctggaatcgcgcggtgttcacggatattgatgcagctgaatcgacaggtgtcggctgggcccatatttcggttcccgtgtcgacggtgcagatgaaggccaagctgggtatgaaccctgagcaggtgactgagctgatccgcaagtctgtcgattacagtctgtgtaaaggattgactgtttccgtaggctttgaggatgcttcaagggcagatgacctgttccttgagcagttgacgagtcagctctatagagatggtatccggcgcttcagatatgccgacacgctgtccgttcaccatcccgctgccatagctgcccgtatagacaggtttgtatcgcgcgtgccacaggatgtggagcttgagattcactgtcataatgattatggcctagcgcttgccaatactttggcagctttgcaggcgggagctgtctgggccagtaccacagtgtcgggccttggggaaagggcaggtaatacggcgctggaggaagtggtgatgtcgtggagggacctatatcaaggaacctgcagtgtccgtcccgaaatgctgaacccgctggctgcactggtgtccaaagcctccaaccgaatcattcccgaaggcaaaccgattgtgggagacatggtattcgcccatgaatccggcatacatatcaacggtctgctaaaggagcgcgccgcctatcaggcccttgatccgaccgagctgggcgctgaccattcgtttgtactcggcaagcattcgggcagaagtgcagttcaatatatgctgaagcaggaaggaatcgaggcaggctccggtgaaatcaagttcctgctggagcggcttcgcctagtcggtgaagatcccaagcgtgtcatccatagcgcggatttaaggcgctggctgcagtattatccggcagagctgccgaaataa

SEQ ID NO: 12 - DNA, 744 bp, Paenibacillus sp.
gtgtcatttgagggacaagttgttattgtaacaggagcggcgcatggcatcgggaaagacgtcgcttttgcctacgctgctcagggtgcccatgttgtatttgcggaccacaatgaggaagagggtgccgcagccgctgcagcagcccgtaacgagggctatcaggccatttttattccttgtgatgtgcggaaggaagaagatattgtagcgctaatccgtgccacggaagaagaatacggtacgattcatacgctaattaacaacgctggcgtgtccaaatggaaatctccatatgagttaacgatagcagaatgggatgacattttgaatacaaacgtacgcagctgttttattgccactagggaagcggctaaggtaatgaaacgaaacgtagaaggtggctctgtggtcaatatgacctctacacgagctttcatgtcagagccggatacagaagcctacgcggcatccaaaggagccattgcggcccttacacacgcgatggctatatcgctaggaaaggacggaattcaggtcaattgcatcagtccagggtggattgaaaccggggattatgagcaactgcgtgcagaagaccatcaacagcatccgtctgggcgagtaggaacccctaaagacatcgcacgggcctgtctgtatttgacacaacccggcaatgattttgtaacaggcacgaatttggtagtagatggtggcatgacacgtaaaatgatttattag

SEQ ID NO: 13 - DNA, 837 bp, Paenibacillus sp.
atgatacaggcggttatttttaatctcgaaagtacattgttagaccccggaggacgaggcttaacccagggaatgcgtgacattttcagatggaaaggtatacaggtaactgaagaacaggtatctcatgggcgaggaatgccgatccgggatcatattgcgaatgtgctgggactttcagaggttcggaaaaaatggctggagtgctttggtgcgcatcctggcagagcggatattgagcatatttatcttgagcttgtccctgttctccgctccttgatccaaggggctgagcctgctatcggtatagacagggcgctgcaggagttgcaggatcgtcgcattcagtcaggtgccactgcatcctgtccgatggaaatgctcagtcacaagtttacgcctgcacagtcaccctatacgttggattgtatggtagccccttgtgaagtttcgcaggggaggccttatccgtggatgatctatgaaattgcacatcgcttgcaggtattcccgcttagcgatattgtgaaagtaggagatacagtagccgatatgcaagaggggcggaacgccggtgtatggacgattggtgtattgaaccataacgaaagcgtgacgaccgcagctcaaacgaccagccatgaagcggacgacagagtggctgaggaacggcgcagacagagtgtatatggcttgaaaagggcaggagcccacatcgtgatgaattcggtcgctgacttaccgcgtattttgcgtgaaatcgaaatgctccagcatacgggaggctatccttcttatacgaaacaggtgaaaacagccgcgctacctccattataa

SEQ ID NO: 14 - DNA, 447 bp, Paenibacillus sp.
atgagtacaacattagaagtagctattttcttatccatgaacggaaaggcaaaagaagctatacatttttacaaaaaacacttcagcgcagaggaattgtttcttgttacctatcaagacatggcccaacgtgacggctccatacagcttacagatgaaaataaaaattatatttctcattcagtcttatccatcggaaaaacaaaagtaatgatagcagaagatacaatgaatcccagtgaagaatatacagtagggaataatacttcattatgtattcaaagtgctgacctagaagaaattaaacgtttttatgatagcttaattacagatgatcgagtgaaaattattgtgcctttatctaataatgtgtttagccaagcctacggtattattgaagacccattcggtattcaaatacaattaatgtttgataatcgattataa

SEQ ID NO: 15 - DNA, 969 bp, Paenibacillus sp.
ttgttcaaaaaagcactgaccttgtgcatgaccttcacactggcagcgggtctagccgcttgtggctccaatgcaagcaataacaacagcgcgtccactccaggtacagatggtacaaaaagcgaaagcacagcctatgttcctaaggagttaaaggtacagttcgtcccttctcaaaatgcagatacgctcgaagccaaagccaaaccgctcgaaaagctgctgggtgacaagcttggaatcccggtgaaagtaacggtatctcctgactacaatacaattgtagaagccatgtcttccaaacaggtagatgtaggatttcttccgccgaatgcttatgtactggcccacgataaccgtaaagcagccgacctgctgttgcaagctcaacgctacggaattgaagatgctacaggcaaggacacgactgaaaaagtaaacttttataaagcaatgatcgtggtgaaaaaggattctccgattcaaagtctagctgatctgaaaggcaagaaggttggctggcaaaatgtaacctcctccgcaggctacgtgtaccctgcagcagagctgaaaaaagctggcgtggacccggacacggacgtgcagggtgtgacaatcaaaggccacgatgctgctattctggcattgctgaacggtcaagttgatgcggtagctgttttccaggatgcacgtaacacggtgaagaaggaaatcccggatgtatttgaaaaaactcgcgtcattcattacacagctaaaattccaaatgacaccgtcagcgtacgttctgatatggatcaagcgtggagagacaagatcagccaagcctttattgacatcgctaaagatcctgcaggcggcgaaatcattaaagagatttacacccatgtaggttatgaaaagggcgacgataaaaacttcgactccgtgcgtgaatacgccgaagccgttggtcaagcggttaagtaa

SEQ ID NO: 16 - DNA, 684 bp, Paenibacillus sp.
atggtcgtcattgtcggcttatccggcgcaggtaagtctaccctgctgcgttccattaaccgactgcatgacattacgtccggcgacattgtcgtgggtggcaggtctgtcacgtctgctggaagcagcgagctacggcgcattcgccgtgatatcggcatgatctttcaaagcttcaatctggttaagcgttcgacggtgctccgcaatgtactttccggccgcgtcggctatcattcaacgctgcgaaccattctcggcctctttccgaaggaagatatcgaactggcactaacggcgctggagcgcgtaaacatccgcgaaaaagcctacagccgggcagatgagctgtctggtggccaacagcagcgcgtatccatcgcccgtgctctggcgcaggaggctaaaatcattctggcggacgagcctgtcgcatcgctggatccgctgaccacccgccaggtaatggatgacctccagcgcatcaaccgtgaaatgcagattacaaccatcgtcaatctgcatttcattgatctggcgcgcgaatatgcaacgcggatcatcggattgcgcgccggagaagtggtctttgacggcacaccggaggaggctacggatgaggcctttgcggagatctacggccgggccatcaagcatgatgagctgctgggagtgggatcatga

SEQ ID NO: 17 - DNA, 855 bp, Paenibacillus sp.
atgaagcccgactctactgcccagccgctctcttcgacgcaatcgtcaccgcatgctggaccgccgcagccaagtaagccgcccgcagggccacctttgccaggacgttacaagcgttatgccacctggatgatttttattgtggtgctggtagcctgctccattcatacggatgccacaccttaccagttgatcgtcggcttgccggagatgggcaaattgttgggcgaaatgtttccacccgactggagctacttgcctaccatctggaaaccgatgctggaaacgatccagatcgccattgtcggcacgacactgggagccatattggcgatccctgtcgcgttgctgtgtgcctataatgtgatgccgcgcaagctgatttcgctgccgatgcgcacgatcctgaatctagtacggacgattcccgatttgctgtttgcctcgatctttgtggcggtgttcggcatcgggccgtttgcaggtatgctcgccctgctcttcttctcctttggcatcatagccaagctgacctttgaggcgattgaagccattgatcccgggccgctggaagccatgaccgctgttggcgcgagccgcattcaggtcatcgcctttggtgtggttccgcaggctcttccttattttgtgtcgtacttgctgtatacctttgaggttaatgtgcgcgcggcttccgttctgggcctggtcggcgctggcggtatcggtctgttgcttgaccgttcccttgggctgttccgatatgatcgggcaagcatcattattctactgaccctcgtcatcgtccttgcgattgattacggcagtaacctgctacggaggaaattattatga

SEQ ID NO: 18 - DNA, 1113 bp, Paenibacillus sp.
atgagttctgtcgtcaagaaaaacattttgcttacccctgggccggcaacgactacagaaagtgtgaaatgttcacaggtggttccggatatttgtcctcgtgaagccgagtttggccaaatcatggagtatatttctacagaactgacccggttggttgctgacccggatgattatgcgaccgtattgtttggcggatcaggtactgcagcggtggaatccatcatcagttcggtcccgaatcacgatgcagtaatcattgtcagcaatggggcctatggaaaacggatgtgcgagatagcgaaggtttatggggtagattatcttgaattcaaaagtccccccgatgatgcgattgatttggctgaattagaaaggtttatccaatcttccgaacgcactgtctcccatctggccgtcgtacatcatgagacgacaactggcctgcttaacaaggtgaaggatatcggagaattgtgcaaaagatatcaaatcgacatgatcgtggatgccatgagctcatttgcggccgttccaattaaattgaaggaaatgaacattgcctacctggcggcaagctccaacaaaaacttacagggaatggccggggtttcgtttgtgatcgccgaaaaaagcaaattggagaatttgaaaggccagaaaccaagaagctattatttgaatttgtatgctcaatataactattttgtggaacatgggcagatgcggtttactcccccagtccaaaccttatatgcgcttagacaagcgattgaggaattaaagcaagagggggtggataaaaggtacgaaaggtacgcgacatcctggaaaaccttaattaacgggttagcccggttaggtctaacctatatcattcccgaagagcatcattccaagattattacttccattcttgaacctcattgtgcaggttatgattttcaatctatgcatgattatttttatagcaaaggcatgatgatttatcccggcaagcttgagaagctaaatacgttccggattgccaatataggtgatattacgtataaggaaatggagttatttttggaattgcttgaacgctatctcagtagcataaaatgcggatga

SEQ ID NO: 19 - DNA, 1161 bp, Paenibacillus sp.
atggacacgaaattgttaggagaagagctgaaaaaattgggatttacctttttcagcggtgttccatgctccttcttgaaaagcttgatcaattatgccataaacgaatgcgattatgttgcagcggctaacgaaggggatgcggtcgccattgcatccggagcttatataggggggaagaaatcggtagtccttatgcaaaattcggggctgaccaacgctgtttcgccattgacctcccttacttacccttttcgtattcctctgcttggatttgtcagcctgcgcggagaaccggggatacctgacgagcctcagcatgaactgatgggccagattacaacgcaaatgctggatttgatggatgtggaatggcagtatctctccaaagatttagaggaggtaagaaatcaattgatccaggctaatgagcaaattgctcggaatcgcccgttcttttttgtcgttaagaaaggcacgttcgacaaagagccattgaaaaaacagcaaacctccatccatttaaatcagatccggcagcatggtcgtgcgaatcatcaaatgcctacgcgctatgatgcattatgtgcgattaactccatgaaagacagccataccgtccagctcgcaacaactggtaaaaccgggcgcgaactatatgaaattgaagatgcagataacaacctgtacatggtaggttctatggggtgcatcggttcttttggactggggttagctctgagccaaccaagcaaaaatattgtcgtgattgacggagacggatctctgttgatgcgtatggggagcctggcaaccaacgggtactaccatacggccaatatgattcatattatgctagataataatgcccatgaatcaacaggcgggcaaagtacggtatcgcataatattgattttatagatattgctgcttcctgtgggtatacaaagtcgattcttgttcacagtctcgaggaactaaaattctttttacaagaatggaaaatgaatagagaacttacgtttttgtgcctgaaaatatccaaagattccaaggatcagcttgctcgtccgcatatgaagcctcacgaagtaaaagaacgtctgcagcgttttattcataacgatcctttgaagggcaaggaagtgggcggttcatga

SEQ ID NO: 20 - DNA, 900 bp, Paenibacillus sp.
atgaaaaaaacaaagcaacttagagacttggtcaattcacaaaagccagagtttatcatggaggctcataatggtctgtcagccaaaattgtggaggaggcaggatttaaaggaatttgggccagtgggttatccatctctgcagcgatgggagtaagggacaacaatgaggcctcctggacgcaggttttggatgttttggaattcatgagtgacgctacctccatcccgattttgttggacggagatacggggtacggcaacttcaacaatgcaaggcgcttggtgaagaaactggagcagcgccatattgcgggggtatgtatcgaagataagcggttccccaaaacgaattctttcattaacggtcaagagcagactttggctgatatcgaagagttttccgggaaaattaaagcgatgaaagatacgcagacggacacggattttacggtggtggccagagtagaggcatttattaccgggagtgggcttaaagtggcgttgcagcgggcggaggcatatcggaaggcaggagccgatgccgtatttatacacagcaaaagatcggatgccgcagagattgaagcgttcatgaaggaatgggctggaagattgccagtagtgatcgtgcctaccaaatactatacgactccaaccatcaggtttcaggagatggcgattagccttgtgatttgggcaaaccataatttgagagcatccattcaagccatgaaacaattatctggccgaatttatcgggatgaaagccttgttcatgtcgaaaaggaagtcgcaactctcgaagaagtattccggcttcaaggggtcgaagagctgcaagctgcggaaaaaaaataccttccatcgtcagaaccggattcgggtgagcgcgtataa

SEQ ID NO: 21 - DNA, 339 bp, Paenibacillus sp.
atgtctaatttaccaaattgtcctgaatgtaattcagagtacacttatgaggatcgcagtcttttcatttgcccggaatgcgctcatgagtggtctaccgagtcagaagcaagcagtgaagacgtaatggttgttaaagatgcgaatggtaacatcctgcaggatggtgactctgtaacggtcattaaagaccttaaagtgaagggaagctcatccgtcttgaaaatcggcaccaaagtaaaaaacatccgtctggtggatggcgatcacgatattgactgcaaaattgatggttttggagcaatgaagctaaaatcggagtttgtaaaaaagaattaa

SEQ ID NO: 22 - DNA, 924 bp, Paenibacillus sp.
atgttgaaaaaatggccgttcattctgatgactctcaccatggtatttgcacttgcagcttgcggatcaggtaacaatgctgcaccacaaggagaagctgctacaggaacagacaataaagcagctgctgaactgacaggaaatattctggctgttggctccactgcacttcaaccattggtagagcaagcgggacaaaaatttatggctgtcgataaatataaaggcattgcagttcaagtacagggcggcggtagcggtactggcttgactcaggtttccggcggacaagcggacatcggtaactccgacgtgtttgcaaaagaaaaactggagagcggcgcagatgagctggtcgatcaccaagtagctgttgtagccatgtcagcggtagcgaatcctaaagtaggcgttacagacctgaaaaaagatcaattggtacaaatctttacaggtaaaatcacgaactggaaagaagttggcggtgcagatcaaaaaatcgttatcgtaaaccgtccaagcagctccggtactcgtgcaacatttgaaaaatacgcattgggacaaaaaacggaagatctgccgggctccattcaagaagattcctccggtactgtgaaaaagctgattgctgaaacaccgggtgcgattggttatctggctctgtcctacatcgacaacacagtaactactttgaaatacgatggcgttgaagctaacgttgaaaatgtagaacaaggtaaatatccagtatgggcttacgagcatatgtacactaaaggtgagccaaaagagcatgtgaaagcattcctggactacattctgtctgatgaaatccaaaaaacggatgtcgtagacttgggatacattccagtagcaggcatgcaagtgaaacgtgatgctgacggtaatatcacgaaataa

SEQ ID NO: 23 - DNA, 933 bp, Paenibacillus sp.
atggaaccaattgaagggaaggcatacatggaacagaataaacgatcccgagttatgaaagaaagacattactgggaagaatggaccggccgcatatatacgtcggtttgtgtcgtttttttagttgcggtcattgtctccatcgtatattttgttgcctccaaaggtgtagctacatttgcggtgaatggagtcagtctgcgagagtttttctttggaaccaaatggagcccggatggcgagcctaagtccttcggcgcgcttccgtttattacgggttcttttgcagtaaccatactggcggctttaattgccagtccgctcagcttatgtgccgctttatttatgacggaaatcgtgccgaaatcgggtaaacgtatccttcaacccgcgattgaattattatccggtattccgtctgttgtatatggctttgttggcttgaccgtgatcgtcccgctgctgcggaacgtgtttggaggacaaggtatcggcattctggcaggctgcctagtgttgtcagtcatgattttgccgacgattacgagtattatggcggatgcgttgtcttccttaccgggggggctgcgtgaatcctcttatgctttgggtgctacccgctggcagacaattgcccgtgttattattccgacgctgttgcctgctcttttaacaggaattattctcggtatggctcgtgccttcggagaagcgttggctgtacagatggtaatcgggaatgctccgcatataccgcattccttgctggaatccgcttcgaccttgaccagcgtcattacacttagcatggggaatacagcaatgggctccgttcataacaatgctctctggagcatggcgcttgttcttctgctcatgacattcattttcgtgctgctggtaaggctgcttgaaaggagaaaccgggtttga

SEQ ID NO: 24 - DNA, 897 bp, Paenibacillus sp.
atgaaagctaaaacgaccgacaagatcgctacatttgtaatctgtttcttcgccttgttcattgttgcacttttggtgggattactgggcttcattttggcccgcggagtcagtcatatttcttttgactttttaaccagtgctccccaaacattacgtgcaggcggtggcattggcccacaactatttaattccgtatttttgttaatcttaactttgattatcaccattccgattggctggggcgcaggtatttacatggcgcaatatgcaaagccggggaaaattacggactttatacgacttgtcgtcgaagtattgtcctcgttcccgtctatcgtcatcggattatttggactactacttatcgtaaatacgtttgattttggcttttctctcttatcaggagccttggctctagcggtattcaacctgccgctgatggttcgtacaacagaacaggcttttcgagctgtgccgaaggagcaaaaggaagctggacttgcgcttggattgtccaagtggaaaattatcacctccattttgttgccagctgcgctgccaagcttgattacgggtaccattttggccgcaggccgtattttcggggaagccgccgcattgctgtttacagcagggatgagtacgccgccgcttgattttaccgattggaatccattgcatgccagatcgcctattaacccgatgcgacctgctgaaacattggctgttcatatttggaaggtcaacagtgagggaattgctcctgactccaaagatattgctgcaggcgcttctgccgtactggtgctgcttgtgcttgcattcaacttgagtgcaagatggttcggaagagttgtataccgccgtctgacagctgccaaacgtatggattag

SEQ ID NO: 25 - DNA, 843 bp, Paenibacillus sp.
atggaacatgtgattacgcgaacggcaaaaaccgccgcaccagcaacatccgttcagtctacgcgttcggaacagcttgcggggcaaccgccgcatccgttcagtactgaggatttgagtatttattatggcagctttgaggctgtgaaaaaagtgaatctggcctttcccgagcagacagtgactgccttgatcgggccctccggctgcggtaaatcgacctttttgcgctcccttaatcggatgaatgatgagattgcgtcttcctctacaacggggcacatctggatggatggacaggatttgacggcaccggaaacggacgtgatcaagctgcgtcagcaaattggcatggtgtggcagcgtcccaaccctttttacaaatccatttacaataatatcgccttcggtcccaaataccgtggggtgcgcaagaagaagcagcttgatgaaatcgtcgaaagcagcctacgtaaagcagcattatgggatgaggtcaaggatcgccttcacgactccgctctggcgctgtctggcgggcaacagcagcggctttgcattgcacgtgcactatccgttcaaccgaaaattctattgcttgacgagccagcctccgcgctggaccctgtgtctacaggcaaggtagaagagctcattactgaattaaaaaaggaactgcgcatcgttattgtgacgcacaatatgcagcaggctgcacggatttccgattatactgcttatttttatatcggaagtctggttgagcatgggaaaacgaatgatattttcacgaatcctgaaaatgagctgactcaggagtacatcatgggtcgatttggttaa

SEQ ID NO: 26 - DNA, 1803 bp, Paenibacillus sp.
atgaaatcgtttcgtttcaggcttaccctgatcatgctgattttgattggcgtatctgtccttgctgctggaatcacgatgggacaaatttttaaaaattcgaatatgaaggtgctagaggagaatatgggacgggagatcgacctgcttcgcgctacattcccttttgtaaatgcagatgccctgtccagcacggattatgtctcttattattctcaaaaggcgaaggaaattgctcgtctgacggaatcgcgggttaccttcattcgcaaggacgggacggtggttggcgattcgttaagtgatccgcgtaagatggaaaaccacgcatcacgtgatgaaatacggcaagccacggcggaaggaatcggtcggacaatacgctacagcgaaactctgcaacgaaacatgctatacgtcgctgaacctgttgtatcggacaagggctttgacgggtatatccgtttgtccatgagccttaaagcggtggaagagggagtgcaacgcggctggacggccatcgggattggtcttgtgctgctgtttattgctgccggattggtgagctatcgtattgctcgcagtctgacctcgcctattgaacatattacaggggtagccaaccgcatatcagggctggattatgacgcaagagtcggtgtacagcgccgggatgaggtagggcaattgggtgaagccattaatcgtatggctgacagtctccaaaaccaaatgaaaacgatacgtgacaatgaggatctcctccagagcgttatgagtaatatgacaggcgggattctaatgattgacgcaagtgagtgtattgcgcttgtaaaccgtgagtctgagcgaatgctaggtgtcgtgggtaaacgggtgacggacaagccgtatcatgagctgaagaagcactatgagctcacaaagctaattgagggcagcatacaaagccgagagaggttgcatggtgaggttcatttgtacaatcccgaggagagactcgtgttgctggacggtgtcccgatgtatgaggatgaaggcggataccgcgggatgctgttccttttacaggatattacggccattcgacggttggaaaatatgcggagcgaatttgtggctaacgtttctcatgagctaaaaacaccgattgccgcggtcaagggctttgccgagacgttactcggtggcggggttaaagatgaagagacggcccgttcatttttgcaaattatatatgatgaaagcgaacgattgaatcggctgatcggtgacattctggagctatccaaaattgaatccaagcgttctccgctggattgctcgccggtgcacgtttcgtcgttcatagaatcattgctggaaaaactgaacaatgttgcggccaaaaaaagaattacgctacacatggatatcccggatgagttgtttatggaggcagatgaagataagcttcagcagattttcttgaaccttttgtctaatggcatcaactatacacttgatggtggcaaggtaaagattaaggttctaacgatacagcgggagaatgacacggaaaaggtcgtatttacagtcagtgatacaggtattggaattccgaagaaggacctgccacgtatctttgagcgcttttaccgggtggacaaaggacgctcgcgcaactctggcggaacggggctgggattgtcgatcgtcaaacacttggtcgatttgcatcatggcaccctttcggtggaaagtgagcttggattgggtaccacatttacagttgaattaccgctattgcaacaggaagaatga

SEQ ID NO: 27 - DNA, 777 bp, Paenibacillus sp.
ttgaccaatgcgcctttaacaggtaaacaggagatgaagttatttatgggacaacgcttgctggttattgaggatgaaccaacgcttgccagattgctatcctacaatttgatacaagaaggcttcgaagtggacacggaggatcatggcagtgccggttttgaaaaagcctccagagagtcctacgatttgatcctgctggatttgatgctgccaggaatgaacggactggacattctgagcagacttcgccatcaagggctgacgacgcctatcattatcttaacagccaaaaacggtgaagccgaggttgtccaagggctgaagtcgggtgcggatgattacatcacgaagccttttggcgtttctgagctgttggcccgcgtgacggctgtgctgcgaagaacttccggcggggatgaaggagtgttgtctgatcagaaagatagctccaaaattcaattgggcgagctggagatttaccccgaaaaatatgaagtcattcttggaggtcagtccatcagcctcagaccaaaggaatttgaggtacttctttatttgtctcgcaagccgggtgtggtattgacccgggatgatctgatgaatgctgtttggggatttgactacattggcggtcaacggaccgtggatgtgcacgtcagttccttacgtaaaaagctggagctagatcccgactcggttcatattgattcgatccgtggagtggggtacaagctggttgtaaataaaaaaagaagcgcttctcatttattataa

SEQ ID NO: 28 - DNA, 1746 bp, Paenibacillus sp.
atgagtgcacaaattcctgaagttaggtcaaccaatgaattacgtgaaaagtggatgaagcctgaagtgattacaggttcggaaattttactccgtagcctattgctggagggagtagattgtgtcttcggatatcctggtggtgcagtgctttatatctatgatgccatgtatggctttaaggatttcaagcatgtcctcactcgtcacgagcaaggagctattcatgcggcagatggttatgcacgggcgagcggtaaagtgggcgtatgtatcgcgacttccggacctggagcgacaaacctggtaactggtattgcgacggcttttatggattcggtgccgttggtggtcatcacgggtaacgttatatcttccctgatcggtacggatgctttccaggaagccgatatcacgggaattacaatgccgattacgaagcacagctatctggtaagagatgtagaggatctgccgcgaatcatccatgaggcattttatattgccaacacagggcggaagggaccggtgctgattgatattccgaaggatatttccgcagcccagacgttgttcgttccacaaacgggaccagtgaccatgcggggttataacccgaaagtgctgcccaacaaaattcaactagacaagctaactcaagctatttccgaagcagaacgtccattcattctggcaggtggaggagttgtatactccggtggacatgaagcactttacgagtttgttcgtaaaacggaaattcctattacgacaacattgctcggattgggtggtttcccaagtggacatgaactgtggacggggatgccggggatgcatggaacatacacctccaatcaggcgattcaacagtcagatctgttgatctgtatcggagcccgcttcgatgatcgggtaacaggcaagttggatgggtttgctcctcaagctaaaatagtccacattgatatcgaccctgctgaaattggaaaaaatgttgcagcggatattcctatcgtgggtgacgtgaaggcagtgctggaattgctgaatcaggatgtgaaacgcgcggatcgagctgatgcatggagagcgcaaattcagcattggaagaatgagaaaccttattcttataaggattctgaaacggtgcttaaaccacaatgggttgttgagttgttggatgaaacgactaagggcggtgcaatcgtaacgacggacgtgggacagcatcaaatgtgggcagcgcagtattacaaattcaatcagccacggtcatgggtcacctctggtggactcggtacgatgggatttggtttcccttctgcaatcggtgctcagatggccaatcccgacagattagtaatatcgattaacggggacggcggtatgcagatgtgttcgcaggaattagccatctgtgcaatcaataacattccggttaaaatcgtaattatcaacaaccaggtgcttggaatggttcgccaatggcaggaattgatctataacaatcggtatagtcacattgacctggctggaagtccggattttgtgaaacttgccgaagcatacggtgtcaaaggactgcgtgctacgaataaggaagaggcacgtcgggcttggcaggaggcgcttgatacacccggaccggtcgttgtcgagtttgtagtcagcaaggaagagaatgtatatccgatggtgacgcaaggttcgacaattgatcaaatgctgatgggggacgagtaa

SEQ ID NO: 29 - DNA, 918 bp, Paenibacillus sp.
atgttcggcaccattcttttggacgttgttttacctatttttgtacttatcggttttggttgtatcatgcaatattattttcgactggatttgtataccttagctaaaatcaatttttactatattacccccgcagttgtgtttacgggtctttataactcagaaatttctttaatgctcatgggtgaggtttcacttttttatgccttgtacatagctattctgtacgtaatcagcactaccgtcgccagatctctgaaatacagccccggaatgcgtggagcttttacaaatagtgtaatgctagataattcaggaaattatggtctgccaatcaatcagttggtgttcaaaggtgacccgttggccatttccgttcaagctctgattatgacctttcaaagctttgtcacatttacatacggcacttttgttgtgcaaaacggcaaggccaatactcgaaaaatattaattaatttttttaaaatgcctgttccctatgcgcttgcgctaggtttgctcctaaatctgctgcatagtccattgcctaatttactggctgaacctttgaattatattagtcagtcgatggttgccgtggctctgcttactttgggagcacaaattgtgcaatatccctttcgcttgcgccggacagccgtgtacatcagtatggtactacgcctgattgctggtccaatggtcggttttatgctcgtcttgctgctgggtcttaaggggattcccgctcaagccttgctcattgcatcgggtatgcctaccggtgttaatacaagtattttggcggaggaatataaaaatgaacccgattttgccgcacaaacagtattactttctacgattgtgaatgtcattacgatgacactgttaatttcagtatcccggcatcttgtttga

SEQ ID NO: 30 - DNA, 960 bp, Paenibacillus sp.
atggctacaggtcatatttttattatcttaattcctattttctttgtcattttaattggttatttcgcaggattgtttaaagcttttaaccctgcttcttctaagggattaaatacactcgtgacgaagtttgctctacctgcacacttgtttattggtattacgacgacaccaagagagacattgattcaaaagtggccctttcttgtggctttgctacttggtgtaatcggcttttatattgtattgctcattgtttatcgcttaattgccaaaaaaggcctgactgaatcctccatgttcgcattgaattcaacacagccttcttttgcattcatgggaattccggttctgggaagcttgttcggaagtgcggcagtcgccatcccgattgcgattactgggatcgtcgttaatgccattcttgatccgcttgcaagtattttgggaacggtcggccagcgcaaaacaacagaatccgacggtactaaaggcaacttatttaaagtcactatgaaatcagtccttcatggactcagcgagccgcttgcctgcgtacccttgtttggtgtcgtattgactctatgtggattccacgctccgaaacttttgcaatccatgtttgatcaaatcggcaatattactgctggtacagccctatttgccattggggtaaccattggtatcaaaaagattacgttcagtccaaaagcacttagcatcgctgtactgaaagccatcgtgatgcctattgttatgctcggtattgctgttctgataggactatcccatgaggatgtaaccaaagctgtactgcttgtcgcttttcccggctcggctgtggccgctatgattgctacccgttatgaaacactggaaacggaagctgcatctgcatttgtcattagctcagtcttatcgctcatttcactacccttgctcatttcacttcttttataa

SEQ ID NO: 31 - DNA, 1056 bp, Paenibacillus sp.
atgtctttactagaaatgattacagcgaccttaacagataacaaaattctcagttctatcgcttccaccgtttttattatccttcttggtttcttttgccgcaaaaaggggatttttaacgcagcagtaggaaagatgttatccaaggttgttctaaccgtggctctaccagcactagcctttaattcttttatgcagggtattaatcctacaatgttaaaacaaggaatgaatgttttaatatggggagttttaatttatattattctcatctttatttctaaaccgttcttcctgagctacaacaaggataaacaggatgttttgcgggtattaaccattttcggttctactactttcttcggaattccaattgtcggagccatttacggtcctaccggagtgatgtacagttccatttttaacatagggtaccgcattttcttatactcctatggctatatcaaaatgagcggcttgaaaatggagcttaaaaatctcaaaaccatgtttttgaatcccatcgttatcgccacgtttctaggcttgtttgtttggttgtttcaaggttatttgccacagatgagtgtagctactgaggatggggccattcagcagttcgcctttctaagaattgatcaaaccgctgtctggctgttcaagcccatgacctacctagctggactagcttctccattagcctggttatcgattggatcgacattgggggagatcagcttcaagtcggcagctactgataagacctcttggtattacagcatagttaaggtttggcttgttcctattgttaacatcgtcttgctggctttgttgactgtatctcatattctgtcagtcgattctgtcgctttagctactattgtcatcatgatggctactcctacagctaccgtagccgcagcctatgccatcagttttgataaggaagccatattaacttctaatgcatcgcttctatctactatcacttctgtcgtcatgattccagtgtggatcattacgctcaatgtcattgataaaattggaattttctaa

SEQ ID NO: 32 - DNA, 1500 bp, Paenibacillus sp.
atggactctttagctgatctctcggaaacccccttagcattggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcctggcgttgttagtgaactgctcacgccggagcaggcggcgagcaagacctatggcgtagcggcacagctcatgcagctgtccgttgtcggcattgcgggacctggagatccgctggccaatgcggaggcaaccttcgataccttccgccgggtccgtgagacagttaaggatgtcatattctgtctcagcacgaatggccttaccttgattaggcatgtcgacaggattgtagagttgggcatttcgcatgtcacgatcacgatcaatgcagtagatccagtggtggggagccgcatttatggatgggtcaacgatgaaggaaaacgctatgcaggagaggaggccgcacggctgttgattgaccgccagctggcaggcttgaagatgctggcttcgagaggcgtattgtgcaaggtgaactcggtgctgattcctgaagtcaatgatgcccatctgccggaggtagccagggtggtcaaggagcacggcgcagtgctgcacaacattatgccgctcatcatcgcacctggtagccggtatgagcaggaagggatgcgggccccccgtccccgtctggtccggcagctgcaggagcaatgtgctgacgcgggggctgtcattatgcgccattgccgtcagtgcagggcggatgcgattggactgctgggcgaagatcgtaatcaggattttacatgggagaacatagcggctgctcctcccatggatgaaggggcaagggcacaatttcagaaagaactggatgagaaggtgagagtgagaatggaacgcaaggagggacaatcgcaccacaaacaaccgtcaaccggggctggctgtagctgcccgttatcgggagataagcctgaagcgagctttacctcgaagccagtcctaatcgctgtggctagtcgtggcggagggaaggtgaatcagcatttcggccgtgccaaggaatttatgatctatgaaagcgacgggaccatcgtaaatttcataggcattcgtaaggtgcaatcctactgccacgggaaagccgattgcaatggggacaaggccgagacgatgaaggagatcctttccatggtgcatgactgtgcattgctgctgtcgtccggcataggcgaagcccccaaagaggcattgcaggaagcaggcgtgctacctattgtatgcggcggggaaattgaggaatccgttctggaatatgtgaaatttctacgttatatgtatcctgtgcagagcaataagggaagtaagcgcaataagggagttaagggcaatcattctgatttacccattgaacattttggaggctga

SEQ ID NO: 33 - DNA, 867 bp, Paenibacillus sp.
atgagacaaattgcgttttacggtaagggcggtatcggcaaatcgacaacctcgcagaatacactggctcagctcgcgaccaaattcaaacaaaaaattatgatcgtaggctgtgatcccaaggcagactccacccgtcttattctgaatacgaaggcccaacagacagtgctacatctggctgctgaaaggggcacggtagaggatttggagctggaggatgttgtccagaagggcttcggtgacattctgaacgtggaatgcggcgggccagagcccggtgtcggctgtgcagggcgtggcatcatcacagccattaattttctggaggaagagggcgcctacgaagggctggatttcgtatcctacgatgtactgggggacgttgtgtgcgggggcttcgccatgccgatccgggagaagaaggcgcaggaaatctacatcgtatgctcaggcgagatgatggctatgtacgctgccaacaatattgcacgcgggatcttgaagtacgccaacagcggcggcgtgcgtttgggcggcttaatctgcaacagccggaacacggacctggaagcggaattgattacagagcttgcgagaagactgaacacgcagatgatccactttttgccgcgtgacaatgttgtgcagcacgcagagctgcgccgtatgaccgttacccaatataacccggaacataagcaggctgcggagtatgaagagctggcaggtaagattttgaataacgatatgctaacggttcccactcccatttccatggaagatctggaggatctgttgatggaatttggcattattgaggatgaagaaaccgcaatcaacaaagctgaggcgtccgggcagtag

SEQ ID NO: 34 - DNA, 1449 bp, Paenibacillus sp.
atgagcagtattgtggataagggaaagcagatcgtagaggagatactggaggtatatcccaagaaggccaagaaggaccggaccaagcattttgagatcgcggatgaggagcttgtgaactgcggaacctgttccattaagtccaacatgaaatcacggcccggcgtcatgacagcaaggggctgtgcttatgcaggctccaagggtgtggtatggggcccgattaaagacatggtgcacattagccatggtcccatcggctgcgggcaatacagttggggtacccgacgcaattatgcgaatggggtattgggaatcgataattttaccgccatgcagattacaagcaattttcaggaaaaggatatcgtgttcggcggagataagaagttggaggtgatctgcagggaaattaaggagatgttcccgttggctaagggcatctccgtgcaatctgaatgtccggtcggactgattggtgatgatatcggggccgtggctaagaagatgacagaggagctgggcattccggtcattcctgtacgctgtgagggctttcgcggggtgagtcagtctctgggccatcacattgccaatgatgctatccgcgattttctgatggggcgccgcgagctaaaggagtccgggccttatgatgtctccattatcggggactacaatatcggcggtgatgcctgggcttcgcgcattttgctggaggaaatgggactgcgggttatagcacagtggtcgggtgacggtacgatcaatgagctggggattgcgcataaatccaagctcaatctgatccattgtcatcgttccatgaattatatgtgcacaacgatggaacaggaatacggaattccctggatggaatataacttcttcggcccgaccaagacgatggagagcctaagagcgattgctgcccgctttgacgagacgattcaggaaaaatgtgagcaggtcatcgcccaatatatgccgcagatggaggcggtcatccgtaaataccgcccacgtctggaaggtaaaaaggtgatgcttctgattggcggactgcgggcaaggcataccatcggtgcctatgaggacctgggtatggaaattgtggctacaggctatgaatttgcccataaggatgattacgaaaagacgtttcccgatgtaaaagaaggcaccattctgtacgatgatccaactgcctatgagctggaggaattggcccagcggctgaatattgacttaatgggcgccggagtcaaggagaaatatgtgtatcacaaaatgggcattcccttccgtcaaatgcactcctgggattacagcgggccttatcatggttttgacggcttcaagatttttgcacgtgatatggatatgaccataaacagtccagtatggagcctgctgccgtcccggcagactgcggaggtgccggtatga

SEQ ID NO: 35 - DNA, 1530 bp, Paenibacillus sp.
atgagcaagcgtctgaacattgtcgatcacaatcagctgtttcggcaggataaatatgtgcgccagcgtgaggagaaacgggccttcgaggccccttgctcgccagaggaggttaccgccaccctggagtacaccaagaccaaggaatacaaggataagaattttgcccgtacagccgtagtcgtgaacccggccaaggcttgtcagccgctgggagcggttatggctgcgctgggcttcgaaaaaacgctccctttcattcatggctcacagggctgtacggcttatttccgtagccatcttgcccgccacttcaaagagcctgttcctgccgtctccacctcgatgaccgaggatgccgccgtattcggtggcatgcgcaacctcattgacggtatagagaactgcattgccttgtatcagccggagatgattgcggtatgcacgacctgtatggcagaggtgatcggagatgacctgtctgccttcctggctaatgcccgtcaggagggagcccttcccgaggatatgccggttccttttgccaatacccccagcttctccggttcacatattacaggctatgacgccatgctgcgctctgtgctggagacgctgtataacaagtcaggccggacgccgcagcctggtcatgaattgaagctgaatgtactgctcgggtttgacgggtatacgggcaattttgcggaaatgcggcgcatactggggatgttcggcgctacgtataccattttgggtgaccacagcagtaattttgattcaggggccactggagagtacagctactattacgggggaacgccgcttaaggatgtgcccgaggccgcagatgctgccggcacgttggcgatccagcagtactctcttcgtaaaacattaggctatatgaagcgaacctggggacagcaggtgtcttccatctccacgccgctgggcatccgcgcgacagattgcttgcttgaagagatcagccgtctgtccggaaaagaaattcccgaggcattgaagcaggagcgcgcccgaattgtggatgccatgatggattcgcatgcttatctgcacggcaaacgagtggctatggcgggagacccggatatgctcatcggcctgattggcttttgtctggagctgggcatggagccagtgcatattgtttgctccaatggggaccggaaatttgagaaggaagcagagctcctgctgaagtccagcccttacggtgcagaagccacagttcattccggtcaggatttgtggcatatgcgttcgctgctgttccaggacccagtggacctggctattggtagctcccatctgaagttcgcggccaaagaggcgaaaattcctttgctccgtgtgggttttccgatcttcgacaggcatcatctgcatcgttatccgattatcggctaccagggtgcgctgaatctgcttacccaattcgtgaataccgtgctggatgtcatggaagagcaggctccggatcatagctttgatctggtgcgctaa

SEQ ID NO: 36 - DNA, 1362 bp, Paenibacillus sp.
atggagccggctgtgtctaacggaaggctggagatatcctgcggcaacaaaattcccaaaagcacgccctgtccccggcctgtgccgggggaggcttcgggtggctgctcctttgacggggcccagattacactgatccccattgcagatgcggctcatctggtgcacgggccgattgcctgtctcggcaatagctgggagagcagaggtagcctgtccagcggcccggagctgtcggcgtatggcttcactactgatcttggagaacaggacatcatttttggtagtgaacagaagctgcatgaatcgattcgctacattgtcagccgctttgctcctcccgctgtgtttgtctatacaacatgtgtcacagccctcaccggtgaagatatcgagggggtctgcaaggctgaatcggagcgactggggacgccgatcattccggtgaacagtccgggatttgtgggcagcaagaatctcgggacccggttggccggagatgtgctgttccagcatattatcggcagcaccgagccggaacagacaacctcccatgatatcaatctcattggggaatacaatattgcgggtgagatgtggcatatcgagcggctgatgcagcaggcgggaatgagtatcctgtcccgaattaccggggacggtcggttccgtgaggtgggatgggcgcatcgtgccaaggccaatatggtcgtatgcagccgggctttgctgggtctggcagtccaaatggagcgtaaatacggcattccttattttgaaggttcattttatggagcaaaggagacgagttattccttacggcagatggcttacctgaccggagatcgtgatgtagagcgacgggtggataagctggccgcacgggaggaaatgaggctatcgctggagctggagccctatcgcaagcagctgaaaggaaagcgggcagtgctctataccgggggtgtaaagagttggtctgtcattacggctttgcaggagctgggcattaaggtagttggtgtaggcacgaacaagagtactgccgaggatgtatcccggattgctgaccgtatcggagatgatgcggaatacatcccggaaggcggcgcccggcagattctcaagaccgtacggagccgcaaggcagacatggtcattgccgggggccggaacatgtatatggcgcttaaggaacagattccttttgtggacatcaatcaagaacgacacaaagcctatgcgggctatgacgggctgttgtctctagcgaaacagcttgtgcatacgctgcagcatccggtatgggagctgaccgccaaattggctccatgggaggaggagacggaatttgccgattaa

SEQ ID NO: 37 - DNA, 1308 bp, Paenibacillus sp.
ttgccgattaaatccgccacgaagcctgtcagtgtcaacccgctcaaggtaggacagcctctgggcggcgtgctggctctgcaagggatgtatcgctcaatgcctttgctgcacggcgcacagggctgctcggccttctccaaggcgctgctgactcgccattttcgagagccgattgccgttcagacctctgcgctgcaagagatggacgttatatttgatgcagaccggaatctggaggaggcgctggatcatatctggtccaagcaccatccagatgtcatcggcgttatcagcacggccctcaccgaggtggcaggcgttgattttcagtccagggtaaaggcgttcaagcgagaacgggcattgaaggacagtctgctgttttctgtatcgctgcctgattttcacggctctctggagacgggctacagcagtacagtagagtcgctaattgatgccgtactcgggttggccggaggcaagtcccccaaaaaacagcgccggacgcagatcaatctgctgccggcttcttatctgactgctggagatgtcatggaaatcaaggatattatcgcttccttcggcctagaggttattacgctccccgatatttccacttccttgtccggtcacctgctgacaggcttttcccctttgacgagaggggggactccgctggattcagcctgccagatgctggagtcttcctgcaccattgccattggcgcgagcatggaacgtccggcgcgcaggctggatcatgccgcaggcattccctaccacttgttcgctggtctgtctggcttggccgcgagcgatgcgttcatacattttctgcagaaaatcagccgtgagccagcccccgttcgctttcgctggcagcgtgaaaatctattggacagcatgctggatgcccatttctattattccggtgcttcggctgtagtggcgctagaaccggatcatatgctgtcgaccgcagcctggctggaggagatgggagtggaactgaagcggctaattacaccctgcagcacgcccgcactgcaaaagacagaacgggaagtctggatcggtgatctggatgatgcagaggagagcgcgcagggcgttgatttgtggatcagcaattcacatggaagaaagggagcggcacgggccggggcctcattcgtaccggcaggcttgccggtgtatgacgagctgggcgcccacacatccgtaagcgtcggataccgtggaaccatggagtgggtgaacaaggtgggtaatctattgcttgccgagagggggagaggagggtga

SEQ ID NO: 38 - DNA, 390 bp, Paenibacillus sp.
gtgaaggttgcatttgcgacggaagacggtgtgcatgtgaatgctcattttgggcagagtcccatgttcactgtattcgaaatccggcactcaggcgtccagttcctggagcatcggcggatagccctggggagcgatgagaacgaggcgggcaagatcgccagccgaattggcctcatcgaggattgtgccttgatcttcctggtacagattggcgcttcggccgccgcacaggttaccaagcggaccattatgcctgtgaaggtggccttcggtagcaccattgaggagcaggtccagcgtctccagaatatgctggctcgtaatccgcccatgtggcttgccaaaatcctgcatgctgaggagggtagcggcaaagccgaatcatga

SEQ ID NO: 39 - DNA, 765 bp, Paenibacillus sp.
atggtacagctgctggaagacagtagatacggacgccagttgaagctgctgggagtggaaggtcagaacaggctaaagcaggctactgttatggttgcaggcatcggaggattgggagggacagcggccatgtacctggccgctgctggagtgggaaagctgatattggctcatgagggcgtaatccatctgcccgatatgaaccggcaggtgctgatggacagcggacgaatcggggaggaacggatggagacggcattgcagcatttgcaccgtattaatccggagacccagcttgaaggccacgccgacagaatcacggaaaaatcctctcgaccatgggtagaagcggcggatatcgtgattgatgcacggtatgactttccggaaagatatgcgctgaacagactatgtgttcgacatggcagaccgatgatagaggcggccatgtacgcctatgaagtatcattgatgaccattgatcccggtaggacggcatgcctggaatgtctctacccggaaggcggacagccttgggaacctctgggattcccggtcctgggagctacctccggtttgattggctgtatggctgcactggaagcggtcaaatggattacagatgcgggcaatctgttcactgaccgcatgtaccgtatgaatgtgctgaatatgagcagctgcaccatagcggtcaaacgcaatccgcgttgtccgtgctgcggaacgggaggaaatacagatgagtcgattgcatatttgtga

SEQ ID NO: 40 - DNA, 1137 bp, Paenibacillus sp.
atgagtcgattgcatatttgtgatacgacacttcgtgacggagaacaggctccgggcgttgccttttcggccgaggaaaaaactgaaattgccatcatgctggactcggcgggggtggagcaggctgagatcggaattccggcaatgggaaaggcagagtgcaggtctattgccaggattgctgctctcggacttcagatgaagctaatgacctggaatcgcgcggtgttcacggatattgatgcagctgaatcgacaggtgtcggctgggcccatatttcggttcccgtgtcgacggtgcagatgaaggccaagctgggtatgaatcctgagcaggtgactgagctgatccgcaagtctgtcgattacggtctgtgtaaaggattgactgtttccgcaggctttgaggatgcttcaagggcagatgacctgttccttgagcagttggcgaatcagctctatagggatggtatccggcgcttcagatatgccgacacgctgtccgttcaccatcccgctgccatagctgcccgtatagacaggtttgtatcacgcgtgccgcaggatgtggagcttgagattcactgtcataatgattatggcctagcgcttgccaatactttggcagctttgcaggcgggagctgtctgggccagtaccacagtgtcgggccttggggaaagggcaggtaatacggcgctggaggaagtggtgatgtcgtggagggacctatatcaaggaacttgcagtgtccgtcccgaactgctgaacccgctggctgcactggtgtccaaagcctccaaccgaatcattcccgaaggtaagccaattgtgggagacatggtattcgcccatgaatccggcatacatatcaacggtctgctaaaggagcgcgccgcctatcaggcccttgatccgaccgagctgggcgctgaccattcgtttgtactcggcaagcattcgggcagaagtgcagttcaatatatgctggagcaggaaggaatcgaggcaggctccggtgaaatcaagttcctgctggagcggcttcgcctagtcggtgaagatcccaagcgtgtcatccatagcgcggatttaaggcgctggctgcagtattatccggcagagctgccgaaataa

SEQ ID NO: 41 - DNA, 750 bp, Paenibacillus sp.
gtgtcatttgagggacaagttgttattgtaacaggagcggcgcatggcatcgggaaagaggtcgcttttgcctacgctgctcagggtgcccatgttgtatttgcggatcacaatgaggaagagggtgccgcagccgctgcagcagcccggaacgagggctatcaggctatttttattccttgtgatgtgcggaaggaagaagatattgtagcgctaatccgtgccacggaagaagaatacggtacgattcatacgctaattaacaacgctggcgtgtccaaatggaaatctccatatgagttaacgatagcagaatgggatgacattctgaatacaaacgtgcgcagctgttttattgccactagggaagcggctaaggtaatgaaacgaaacgcagaaggtggctctgtggtcaatatgacctctacacgagctttcatgtcagagccggatacagaagcctacgcggcatccaaaggagccattgcggcccttacacacgcgatggctatatcgttaggaaaggacgggattcaggtcaattgcatcagtccagggtggattgaaaccggagattttgagcaactgcgtacagaagaccatcaacagcatccgtctgggcgagtaggaacccctaaagacatcgcacgggcctgcctgtatttgacacaacccggcaatgattttgtaacaggcacgaatctggtagtagatggtggcatgacacgtaaaatgatttacgagccataa

SEQ ID NO: 42 - DNA, 837 bp, Paenibacillus sp.
atgatacaggcggttatttttaatctcgaaagtacattgttagaccccggaggacgaggcttaacccagggaatgcgtgacattttcagatggaaaggtatacaggtaactgaagaacaggtatctcatgggcgaggaatgccgatccgggatcatattgcgaatgtgctgggactttcagaggttcggaaaaaatggctggagtgctttggtgcgcatcctggcagagcggatattgagcatatttatcttgagcttgtccctgttcttcgttccttgatccaaggggctgagcctgctatcggtatagacagggcgctgcaggagttacaggatcgtcgcattcagtcaggtgccactgcatcctgtccgatggaaatgctcagtcacaagtttacgcctgcacagtcaccctatacgttggattgtatggtagccccttgtgaagtttcgcaggggaggccttatccgtggatgatctatgaaattgcacatcgcttgcaggtattcccgcttagcgatattgtgaaagtaggagatacagcagccgatatgcaagagggacggaacgccggtgtatggacgattggtgtattgaaccataacgaaagcgtgacgaccgcagctcaaacgaccagccatgaagcggacgacagagtggctgaggaacggcgcagacagagtgtatatggtttgaaaagggcaggagcccacatcgtgatgaactcggtcgctgacttgccacgtattttgcgtgaaattgaaatgctccaacatacgggaggatatccctcttatatgaagcaggtaaaaacagcgttaatgccaccatcgtga

SEQ ID NO: 43 - DNA, 447 bp, Paenibacillus sp.
atgagtacaacactggaagtagctattttcttatccatgaacggaaaggcaaaggaagctatacaattttacaaaaaacacttcagcgcagaggaattgtttcttgttacctatcaagacatggcccaacgtgacagctccatacagcttacggatgaaaataaaaattatatttctcattcagtcttatccatcggaaaaacaaaagtaatgatagcagaagatacaatgaatcccagtgaagaatatacagtagggaataatacttcattatgtattcaaagtgctgacctagaagaaattaaacgtttttatgatagcttaattacagatgatcgcgtgaaaattattgtgcctttatctaataatgtgtttagccaagcctacggtattattgaagacccgttcggtattcaaatacaattaatgtttgataatcgattataa

SEQ ID NO: 44 - DNA, 969 bp, Paenibacillus sp.
ttgttcaaaaaagcactgaccttgtgcatgaccttcacactggcagcgggtctagccgcttgtggctccaatgcaagcaataacaacagcgcgtccactccgggtacagatggctctaaaaacgaaagcacagcctatgttcctaaggagttaaaggtacagttcgttccttctcaaaatgcagatacgctcgaagccaaagccaaaccgctcgaaaatctactgggtgacaagcttggaatcccggtgaaagtaactgtatctcctgactacaatacgattgtggaagccatgtcttccaaacaggtagatgtaggattccttccgccgaatgcttatgtactggcccacgataaccgtaaagcagccgacttgctgttgcaagctcaacgctacggaattgaagatgctacaggcaaggacacgactgaaaaagtaaacttttataaagcgatgatcgtggtgaaaaaggattctccgattcaaagtctagctgatctgaaaggcaagaaggttggctggcaaaatgtaacctcctccgcaggctacgtgtaccctgcagcagagctgaaaaaagctggcgtggacccggatacggatgtgcagggtgtgacaatcaaaggccacgatgctgctattctggcgttgctgaacggtcaagttgatgcggtagctgttttccaggatgcacgtaacacggtgaagaaggaaatcccggatgtatttgaaaaaactcgcgtcattcattacacagctaaaattccaaatgacaccgtcagcgtacgttctgatatggatcaagcgtggagagacaagatcagccaagcctttattgacatcgccaaagatcctgcaggcggcgaaatcattaaagagatttacacccatgtaggttatgaaaagggcgacgataaaaacttcgactccgtgcgtgaatacgccgaagccgttggtcaagcggttaagtaa

SEQ ID NO: 45 - DNA, 684 bp, Paenibacillus sp.
atggtcgtcatcgtcggcctatccggtgcaggtaagtctaccctgctgcgttccattaaccgactgcatgacattacgtccggcgacattgtcgtgggtggcaggtccgtcacatctgctggaagcagcgagctgcggcgcattcgccgtgatatcggcatgatctttcaaagcttcaatctggttaagcgttcgacggtgctccgcaatgtactttccggccgcgtcggctatcattcaacactgcggaccattctcggcctctttccgaaggaagatatcgaactggcactaacagcgctggagcgcgtgaacatccgcgaaaaagcctacagccgggcagatgagctgtccggtggccaacagcagcgcgtatccatcgcccgcgctctggcgcaggaggctaaaatcattctggcggacgagcctgtcgcatcgctggatccgctgaccacccgccaggtaatggatgacctccagcgcatcaaccgtgaaatgcagattacgaccatcgtcaatctgcatttcattgatctcgcgcgcgaatatgcaacgcggatcatcggattgcgcgccggagaagtggtctttgacggcacaccggaggaggctacggatgaggcctttgcggagatctacggccgggccatcaagcatgatgaactgctgggagtaggatcatga

SEQ ID NO: 46 - DNA, 855 bp, Paenibacillus sp.
atgaagcccgactctactgcccagccgctctcttcgacgcaaccgtcaccgcatgctggaccgccgcagccaggcaagacgcccgcagggccacctttgccaggacgttacaaacgttatgccacctggatgatttttatcgtggtgctggtagcctgctccattcatacggatgccacaccttaccagttgatcgttggcttgccggagatgggcaaattgttgggcgaaatgtttccacccgactggagctacttgcctaccatctggaaaccgatgctggaaacgatccagatcgccattgttggcacgacactgggagccatattggcgatccctgtcgcgttgctgtgtgcctataatgtgatgccgcgcaaactgatttcgctgccgatgcgcacgatcctgaatctagtacggacgattcccgatttgctgtttgcctcgatctttgtggcggtgttcggcatcgggccgtttgcggggatgctcgccctgctcttcttctcctttggcatcatagccaagctgacctttgaggcgattgaagccattgatcccgggccgctggaagccatgaccgctgttggcgcgagccgcattcaagtcatcgcctttggtgtggttccgcaggctcttccttattttgtgtcgtacttgctgtataccttcgaggttaatgtgcgcgcggcttccgttctgggcctggtcggggctggcggtatcggtctgttgcttgaccgttcccttgggctattccgatatgatcgggcaagcatcattattctactgaccctcgtcatcgtccttgcgattgattacggcagtaacctgctacggaggaaattattatga

SEQ ID NO: 47 - DNA, 1113 bp, Paenibacillus sp.
atgagttctgtcgtcaagaaaaacattttgcttacccctgggccggcaacgactacagaaagtgtgaaatgttcccaggtggttccggatatttgtcctcgtgaagccgagtttggccaaatcatggagtatatttctacagaactgacccggttggttgctgacccggatgattatgcgaccgtattgtttggcggatcaggtactgcagcggtggaatccatcatcagttcggtcccgaatcacgatgcagtaatcattgtcagcaatggggcctatggaaaacggatgtgcgagatagcgaaggtttatggagtggattattttgagttcaaaagttctcccgatgatgctattgatttggccgaattagaaaggtatatccaatcttccgaacgcactgtctcccatctggccgtcgtacatcatgagacgacaactggcctgcttaacaaggtgaaggatatcggagaattgtgcaaaagatatcaaatcgacatgatcgtggatgccatgagctcatttgcggccgttccaattcaattgaaggaaatgaacattgcctacctggcggcaagctccaacaaaaacttacagggaatggccggggtttcgtttgtgatcgccgaaaaaagcaaattggagaatttgaaaggccagaaaccaagaagctattatttgaatttgtatgctcaatataactattttgtggaacatgggcagatgcggtttactcccccagtccaaaccttatatgcgcttagacaagcgattgaagaattaaagcaagagggggtggataaaaggtacgaaaggtacgcgacatcctggaaaaccttaattaacgggttagcccggttaggtctaacctatatcattcccgaagagcatcattccaagattattacttccattcttgaacctcattgtgcaggttatgattttcaatctatgcatgattatttttacagcaaaggcatgatgatttatcccggcaagcttgagaagctaaatacgttccggattgccaatataggtgatattacgtataaggaaatggagttatttttggaattgcttgaacgctatctcagtagcataaaatgtggatga

SEQ ID NO: 48 - DNA, 1161 bp, Paenibacillus sp.
atggacacgaaattgttaggagaagagctgaaaaaaataggatttacctttttcagcggtgttccatgctccttcttgaaaagcttgatcaattatgccataaacgaatgcgattatgttgcagcggctaacgaaggggatgcggtcgccattgcatccggagcttatataggggggaagaaatcggtagtccttatgcaaaattcggggctgaccaacgctgtttcgccattgacctcccttacctacccttttcgtattcctctgcttggatttgtcagcctgcgcggagaaccggggatacctgacgagcctcagcatgaactgatgggccaaattacaacgcaaatgctggatttgatggatgtggaatggcagtatctctccaaggatctagaggaggtaaaaaatcaattgatccaggctaatgagcaaattgcccggaatcgcccgttcttttttgtcgttaagaaaggcacgttcgacaaagagccattgaaaaaacagcaaacctccatccatttaaatcagatccggcagcatggtcgtgcgaatcatcaaatgcctacgcgctatgatgcattatgtgcgattaactccatgaaagacagccataccatccagctcgcaacaactggtaaaaccgggcgcgaactatatgaaattgaagatgcagataacaacttgtacatggtaggttctatgggatgcatcggttcttttggactggggttagctctgagccaaccaagcaaaaatattgtcgtgattgacggagacggatctctgttgatgcgtatggggagcctggcaaccaacgggtactaccatacggccaatatgattcatattatgctagataataatgcccatgaatcaacaggcgggcaaagtacggtatcgcataatattgattttatagatattgctgcttcctgtgggtatacaaagtcgattcttgttcacagtctcgaggaactaaaagtctttttacaagaatggaaaatgaatagagaacttacgtttttgtgcctgaaaatatccaaagattccaaggatcagcttgctcgtccgcatatgaagcctcacgaagtaaaagaacgtctgcagcgttttattcataacgatcctttgaagggcaaggaagtgggcggttcatga

SEQ ID NO: 49 - DNA, 900 bp, Paenibacillus sp.
atgaaaaaaacaaagcaacttagagacttggtcaattcacaaaagccagagtttatcatggaggctcataatggtctgtcagccaaaattgtggaggaggcaggatttaaaggaatttgggccagtgggttatccatctctgcagcgatgggagtaagggacaacaatgaggcctcctggacgcaggttttggatgttttggaattcatgagtgacgctacctccatcccgattttgttggacggagatacggggtacggcaacttcaacaatgcaaggcgcttggtgaagaaactggagcagcgccatattgcgggggtatgtatcgaagataagcggttccccaaaacgaattctttcattaacggtcaagagcagactttggctgatatcgaagagttttccgggaaaattaaagcgatgaaagatacgcagacggacacggattttacggtggtggccagagtagaggcatttattaccgggagtgggcttaaagtggcgctgcagcgggcggaggcatatcggaaggcaggagccgatgccgtatttatacacagcaaaagatcggatgccgcagagattgaagcgttcatgaaagaatgggctggaagattgccagtagtaatcgtgcctaccaaatactatacgactccaaccatcaggtttcaggagatggggattagccttgtgatttgggcaaaccataatttgagagcatccattcaagccatgaaacaattatctggccgaatttatcgggatgaaagccttgttcatgtcgaaaaggaagtcgcaaccctcgaagaagtattccggcttcaaggggtcgaagagctgcaagctgcggaaaagaaataccttccatcgtcagaaccggattcgggtgagcgcgtataa

SEQ ID NO: 50 - DNA, 339 bp, Paenibacillus sp.
atgtctaatttaccaaattgtcctgaatgtaattcagagtacacttatgaggatcgcagtcttttcatttgcccggaatgcgctcatgagtggtctaccgagtcagaagcaagcagtgaagacgtaaaggtcgtcaaagatgcgaatggtaacatcctgcaggatggtgactctgtaactgtcattaaagaccttaaagtgaagggaagctcatccgtcttgaaaatcggtaccaaagtaaaaaacatccgtctggtggatggcgatcacgatattgactgcaaaattgatggttttggagcaatgaagctaaaatcggagtttgtaaaaaagaattaa

SEQ ID NO: 51 - DNA, 924 bp, Paenibacillus sp.
atgttgaaaaaatggccgttcattctgatgactctcaccatggtatttgcacttgcagcttgcggatcaggtaacaatgctgcaccacaaggagaaactgctacaggaacagacaataaagcagctgctgaactgacaggaaatattctggctgttggctccactgcacttcaaccattggtagagcaagcgggacaaaaatttatggctgtcgataaatataaaggcattgcagttcaagtacagggcggcggtagcggtactggcttgactcaggtttccggcggacaagcggacatcggtaactccgacgtgtttgcaaaagaaaaactggagagcggcgcagatgagctggtcgatcaccaagtagctgttgtagccatgtcagcggtagcgaatcctaaagtaggcgttacagacttgaaaaaagatcaattggtacaaatctttacaggtaaaatcacgaactggaaagaggttggcggcgcggatcaaaaaatcgttatcgtgaaccgtccaagcagctccggtactcgtgcaacatttgaaaaatatgctttgggacaaaaaacggaagatctgccgggctccattcaagaagattcctccggtactgtgaaaaagctgattgctgaaacaccgggtgcgattggttatctggctctgtcctacatcgacaacacagtaactactttgaaatacgatggcgttgaagctaacgttgaaaatgtagaacaaggtaaatatccagtatgggcttacgagcatatgtacactaaaggtgagccaaaagagcatgtgaaagcattccttgactacattctgtctgatgaaatccaaaaaacggatgtcgtagacttgggatacattccagtagcaggtatgcaagtgaaacgtgatgctgacggtaatatcacgaaataa

SEQ ID NO: 52 - DNA, 897 bp, Paenibacillus sp.
atgaaagctaaaacgaccgacaagatcgctacatttgtaatctgtttcttcgccttgttcattgttgcacttttggtgggattactgggcttcattttggcccgcggagtcagtcatatttcttttgactttttaactagtgctccccaaacattacgtgcaggcggtggcattggcccacaactatttaattccgtatttttgttaatcttaactttgattatcaccattccgattggctggggcgcaggtatttacatggcgcaatatgcaaagccagggaaaattacggactttatacgacttgtcgtcgaagtattgtcctcgttcccgtctatcgtcatcggattgtttggactactacttatcgtaaatacgtttgattttggcttttccctcttatcaggagccttggctttggccgtattcaacctgccgctgatggttcgcacaactgagcaggctttccgagctgtgccgaaggagcaaaaggaagctggacttgcgcttggattgtccaagtggaaaatcatcacctccattttgttgccagctgcgctgccaagcttgattacgggtaccattttggccgcaggccgtattttcggggaagccgctgcattgctgtttacagcagggatgagtacgccgccgcttgattttaccgattggaatccattgcatgccagatcgcctattaacccgatgcgacctgctgaaacattggctgttcatatttggaaggtcaacagtgaaggcattgctcctgactccaaagatattgccgcaggcgcttctgcagtattggtactgctcgtacttgtgttcaacctgagtgcaagatggttcggaagagttgtataccgccgcctgacagcagccaaacgaatggattag

SEQ ID NO: 53 - DNA, 897 bp, Paenibacillus sp.; identical to SEQ ID NO: 52

SEQ ID NO: 54 - DNA, 843 bp, Paenibacillus sp.
atggaacatgtgattacgcgaacggcaaaaaccgccacaccagcaacatccgttcagtctacgcgttcggaacagcttgcggggcaaccaccgcatccgttcagtactgaggatttgagtatttattatggcagctttgaggctgtgaaaaaagtgaatctggcctttcccgagcagacagtgactgccctgatcgggccttccggctgcggtaaatcgacctttttgcgctcccttaatcggatgaatgatgagattgcgtcttcctctacaacggggcacatctggatggatggacaggatttgacggcaccggaaacggacgtgatcaagctgcgtcagcaaattggcatggtgtggcagcgtcccaaccctttttacaaatccatttacaataatatcgccttcggtcccaaataccgtggggtgcgcaagaagaagcagcttgatgaaatcgtcgaaagcagcctacgtaaagcagcattatgggatgaggtcaaggatcgccttcacgattccgctctggcgctgtctggcgggcaacagcagcggctttgcattgcacgtgcactatccgttcaaccgaaaattctattgcttgacgagccagcctccgcgctggaccctgtgtctacaggcaaggtagaagagctcattaccgaattaaaaaaagaactgcgcatcgttattgtgacgcacaatatgcagcaggctgcacgaatttccgattatactgcttatttttatatcggaagtctggttgaacatgggaaaacgaacgatattttcacgaatcctgaaaatgagctgactcaggagtacatcatgggtcgatttggttaa

SEQ ID NO: 55 - DNA, 1803 bp, Paenibacillus sp.
atgaaatcgtttcgtttcaggcttaccctgatcatgctgattttgattggcgtatctgtccttgctgctggaatcacgatgggacaaatttttaaaaattcgaatatgaaggtgctagaggagaacatgggacgggagatcgacctgcttcgcgctacattcccttttgtaaatgcagatgccctatccagcacggattatgtctcttattattctcaaaaggcgaaggaaattgctcgtctgacggaatctagggtgaccttcattcgcaaggacgggacggtggttggcgattcgttaagcgatccgcgtaagatggaaaaccacgcatcacgtgatgaaatacggcaagccacggcggaaggaatcggtcggacaatacgctacagcgaaaccttgcagcggaacatgctatacgttgcagagcctgttgtatcggacaagggatttgacggatatatccgtttgtccatgagtctgaaagcggtggaggagggagtacggcgcggctggacggccatcgggattggtcttgtgttgctgtttattgctgctggattggtgagctatcgtattgctcgcagcctgacctcgcctattgaacatattacaggggtagccaaccgcatatcaggactggattatgacgcaagagtcggtgtacagcgccgggatgaggtagggcaattgggtgaagccattaatcgtatggctgacagtctccaaaaccaaatgaaaacgatacgtgacaatgaggatctcctccagagcgttatgagtaatatgacaggcgggattctaatgattgatgcaagtgagtgtattgcgcttgtaaaccgtgagtctgagcgaatgctcggtgtcgtgggtaaacgggtgacggacaagccgtatcatgagctgaagaagcactatgagctcacaaagctaattgagggcagcatacaaagccgagagaggctgcatggtgaggttcatttgtacaatcccgaggagagactcgtgttgctggacggtgtcccgatgtatgaggatgaaggcggataccgcgggatgctgttccttttacaggatattacggccattcgacggttggaaaatatgcggagcgaatttgtggctaacgtttctcatgagctaaaaacaccgattgccgcggtcaagggctttgccgagacgttactcggtggtggggttaaagatgaagagacggcccgttcatttttgcaaattatatatgatgaaagcgaacgattgaatcggctgatcggtgacattctggagctatccaaaattgaatccaagcgttctccgctggattgctcgccggtgcacgtttcgtcgttcatagaatcattgctggaaaaactgaacaatgttgcggccaaaaaaagaattacgctacacatggatatcccggatgagctgtttatggaggcagatgaagataagcttcagcagattttcctgaaccttttgtctaatggcatcaactatacacttgatggtggcaaggtaaagattaaggttctaacgatacagcgggagaatgacacggaaaaggtcgtatttacagtcagtgatacaggtattggaattccgaagaaggacctgccacgtatctttgagcgcttttaccgggtggacaaaggacgctcgcgcaactctggcggaacggggctgggattgtcgatcgtcaaacacttggtcgatttgcatcatggcaccctttcggtggaaagtgagcttggattgggtaccacatttacagttgaattaccgctattgcaacaggaagaatga

SEQ ID NO: 56 - DNA, 732 bp, Paenibacillus sp.
atgggacaacgcttgctggttattgaggatgaaccaacgcttgccagattgctatcctacaatttgatacaagaaggcttcgaagtggacacggaggatcatggcagtgccgggtttgaaaaagcttccagagagtcctacgatttgatcctgctggatttgatgttgccgggaatgaacggactggacattctgagcagacttcgtcatcaggggctgacgacgcctatcattatcttaacggccaaaaacggtgaagccgaggttgtccaagggctgaagtcgggtgcggatgattacatcacgaagccttttggcgtttctgagctgttggcccgcgtgacggctgtgctgcgaagaacttccggcggggatgaaggagtgttgtctgatcagaaagatagctccaaaattcaattgggcgagctggagatttaccctgaaaaatatgaagtcattcttggaggtcagtccatcagcctcagaccaaaggaatttgaggtgcttctttatttgtcccgcaagccgggtgtggtattgacccgagatgatctgatgaatgctgtttggggttttgactacattggcggtcaacggaccgtggatgtgcacgtcagttccttacgtaaaaagctggagctagatcccgactcggttcatattgattcgatccgtggagtgggatataagctggttgtaaataaaaaaagaagcgcttctcatttattataa

SEQ ID NO: 57 - DNA, 1746 bp, Paenibacillus sp.
atgagtgcacaaattcctgaagttaggtcaaccaatgaattacgtgaaaagtggatgaagcctgaagtgattacaggttcggaaattttactccgtagcctgttgctggagggagtagattgtgtcttcggatatcctggtggtgcagtgctttatatctatgatgccatgtatggctttaaggatttcaagcatgtcctcactcgtcacgagcaaggagctattcatgcggcagatggttatgcacgggcgagcggtaaagtgggcgtatgtatcgcgacttccggacctggagcgacaaacctggtaactggtattgcgacggcttttatggattcggtgccgttggtggtcatcacgggtaacgttatatcttccctgatcggtacggatgctttccaggaagccgatatcacgggaattacaatgccgattacgaagcacagctatctggtaagagatgtagaggatctgccgcgaatcatccatgaggcatttcatattgccaacacagggcggaagggaccggtgctgattgatattccgaaggatatttccgcagcccagacgttgttcgttccacaaacgggaccagtgaccatgcggggttataacccgaaagtgctgcccaacaaaattcaactagacaagctaactcaagctatttccgaagcagaacgtccattcattctggcaggtggaggagttgtatactccggtggacatgaagcactttacgagtttgttcgtaaaacggaaattcctattacgacaacattgctcggattgggtggtttcccaagtggacatgaactgtggacggggatgccggggatgcatggaacatacacctccaatcaggcgattcaacagtcagatctgttgatctgtatcggagcccgcttcgatgatcgggtaacaggcaagttggatgggtttgctcctcaagctaaaatcgtccacattgatatcgaccctgctgaaattggaaaaaatgttgcagcggatattcctatcgtgggtgacgtgaaggcagtgctggaattgctgaatcaggatgtgaaacgtgcggatcgagctgatgcatggagagcgcaaattcagcattggaagaatgagaaaccttattcttataaggattctgaaacggtgcttaaaccacaatgggttgttgagttgttggatgaaacgaccaagggcggcgcaatcgtaacgacggacgtgggacagcatcaaatgtgggcagcgcagtattacaaattcaatcagccacgctcatgggtcacctctggtggactcggtacgatgggatttggtttcccttctgcaatcggtgctcagatggccaatcccgacagattagtaatatcgattaacggggacggcggtatgcagatgtgttcgcaggaattagccatctgtgcaatcaataacattccggttaaaatcgtaattatcaacaaccaggtgcttggaatggttcgccaatggcaggaattgatctatgacaatcggtatagtcacattgacctggctggaagtccggattttgtgaaacttgccgaagcatacggtgtcaaaggactgcgtgctacgaataaggaagaggcacgtcgggcttggcaggaggcgcttgatacacccggaccggtcgttgtcgagtttgtagtcagcaaggaagagaatgtatatccgatggtgacgcaaggttcgacaattgatcaaatgctgatgggggacgagtaa

SEQ ID NO: 58 - DNA, 918 bp, Paenibacillus sp.
atgttcggcaccattcttttggacgttgttttacctatttttgtacttatcggttttggttgtatcatgcaatattattttcgactggatttgtataccttagctaaaatcaatttttactatattacccccgcagttgtgtttacgggtctttataactcagaaatttctttaatgctcatgggtgaggtttcacttttttatgccttgtacatagctattctgtacgtaatcagcactaccgtcgccagatctctgaaatacagccccggattgcgtggagcttttacaaatagtgtaatgctagataattcagggaattatggtctgccaatcaatcagttggtgttcaaaggtgacccgttggccatttccgttcaagctctgattatgacctttcaaagctttgtcacatttacatacggcacttttgttgtgcaaaacggcaaggccaatactcgaaaaatattaattaatttttttaaaatgcctgttccctatgcgcttgcgctaggtttgctcctaaatctgctgcatagtccattgcctaatttactggctgaacctttgaattatattagtcagtcgatggttgccgtggctctgcttactttgggagcacaaattgtgcaatatccctttcgtttgcgccggacagccgtctacatcagtatggtattacgcctgattgttggtccaatggtcggttttatgctcgtcttgctgctgggtcttaaggggattcccgctcaagccttgctcattgcatcgggtatgcctaccggcgttaatacaagtattttggcggaagaatataaaaatgaacccgattttgccgcacaaacagtattactttctacgattgtgaatgtcattacgatgacactgttaatttcagtatcccggcatcttgtttga

SEQ ID NO: 59 - DNA, 960 bp, Paenibacillus sp.
atggctacaggtcatatttttattatcttaatccccattttctttgtcattttaattggttatttcgcaggattgtttaaggcttttaaccctgcttcttctaaaggattaaatacactcgtaacgaagtttgctctgcctgcacacttgtttattggtattacgacgacaccaagagagacgttgattcaaaagtggccctttcttgtggctttgctacttggtgtgatcggcttttatattgttttgctcattgtttatcgcttaattgccaaaaaaggcctgactgaatcctccatattcgcattgaattcaacacagccttcttttgcattcatgggaattccggttttgggaagcctgttcggaagtgcggcagtcgccatcccgattgcgattactgggatcgtcgttaatgccattcttgatccgcttgcaagtattttgggaacggtcggccagcgcaaaacaacagaatccgacggcactaaaggcaacttatttaaagtcactatgaaatcagtccttcatggactcagcgagccgcttgcctgcgtacccttgtttggtgtcgtattgactctatgtggattccacgctccgaaacttttgcaatccatgtttgatcaaatcggcaatattactgctggtacagccctatttgccattggggtaaccattggtatcaaaaaaattacgttcagtccaaaagcacttagcatcgctgtgctgaaagccatcgtgatgcctattgttatgctcggtattgctgttctgataggactatcccatgaagatgtaaccaaagccgtactgcttgtcgcttttcccggctcggctgtggccgctatgattgctacccgttatgaaacactggaaacggaagctgcatctgcatttgttattagctcagtcttatcgcttatttcattacccttgctcatttcacttcttttataa

SEQ ID NO: 60 - DNA, 1056 bp, Paenibacillus sp.
atgtctttactagaaatgattacagcgaccttaacagataacaaaattctcagttctatcgcttccaccgtttttattattcttcttggtttcttttgccgcaaaaaggggatttttaacgcagcagtaggaaagatgttatccaaggttgttctaaccgtggctctaccagcactagcctttaattctttcatgcaggatattaatcctacaatgttaaaacaaggcatgaatgttttaatatggggaattctaatttatattattcttatctttatttctaaaccattcttcctgagctacaacaaggataaacaggatgttttgcgtgtgttaaccattttcggttctactactttcttcggaattccaattgtcggagccatttacggtcctaccggagtgatgtacagttccatttttaacatagggtaccgcattttcttatactcctatggctatatcaaaatgagcggcttgaaaatggagcttaaaaatctcaaaaccatgtttttgaatcccatcgttatcgccacgtttctaggcttgtttgtttggttgtttcaaggttatttgccacagatgagtgtagctacagaggatggggccattcagcaattcgcctttctaagaattgatcaaaccgctgtctggctgttcaagcccatgacctacctagctggactagcttctccattagcctggttatcgattggctcgacattgggggagatcagcttcaagtcggcagctactgataagacctcttggtattacagcatagttaaggtttggcttgtacctattgttaacatcgtcttgctggctttgttgactgtatctcatattctgtcagtcgattctgtcgctttagctactattgtcattatgatggctactcctacagctaccgtagccgcagcctatgccatcagttttgataaggaagccatattaacttctaatgcatcgcttctatctactatcacttctgtcgtcatgattccagtgtggatcattacgctcaatgtcattgataaaattggaattttctaa

SEQ ID NO: 61 - DNA, 1461 bp, Paenibacillus sp.
gtgcttattacgcatacggattgccgtattatctcacctgaaggaattatgactagggagcgtctggagcgagacgttacgatcttctcccggatgttgacgctcagaggaattcccgaagacggtagagtgattctgaaagcggccaattcctattggtttgttgtgtctctgttttccctgtgtaatattgctgtttcggtgactgtggtggacgagcagactgtgctcgatgaggtggcccaaatctatgatgaagcgggtgcctgttgcatccttacggattccgatttagaacttcctcatgctctcgttattctgattcaagatctgattcaagaagaagcagtacacccggattgggtgggagagccaaagcttgaagccctcgactttaacaagtggtgcgagaggccagatgcgctcattctgtattcttcaggtacgacgggaaaacccaagggaatcgtgaaagctggttcagcttttatggagaatatcaggcattccatacacgccatgaactacctaccgtctgaccatatgctccccgttgtgcctttctctcatttttatggaatctcacttatcttctcctggtggttaacctcatgttctctcatcatctgcaatcccaaaaacctctggagcgtcattgcaagtattacgaaagaccgggcgacggtcgtagacgctaacccctctgctttttacaccttgttgcgtatgctaaaccgaaaacccgaacaactggaaatggtcaaggaagctccggtgcgcatgtggtgcgtaggcggttccccacttacccaagacctcgaagagaaatttaacaacatatttggacagcccctgctgaatggttacggcctttctgaactgggcaacgttacgctggggacgctagagtgtccgaagggttgtggcctacccttacctggtgtggatctgagaatactagattctacaggcaaccagcaggcggacggtatcgtaggcgaagtgtggatacgcagtgctggatgcatggaaggttacttgaatcgtccagacctgacgcaatccgttctacaggacggctggttcaagacgggggatctgggctatctggatgacggaatgctgtatgttattggtcgcagcggtaagacggtcaaccggatgggctacatggtatctccggtctatattgaggaccggattggttcgcttggttaccgatcctgcgtgatcaccttggaggatgaggctaaagggacattgcttgttgcattcatcgaaggtgaatcctcgcaagccttgtctgtactccgcaaagaaataaatcgtgtacttccttcatacatgtttcccgatcttttgcttccgctcgaccattttcctttaaatcgcaatggaaaagtagatcgtctggagatggagcgtattgccctggagaaagttgcatctcgcaaagtatag

SEQ ID NO: 62 - DNA, 1224 bp, Paenibacillus sp.
atgagtataaatccagaattggcagaaaacacactgtttttgaaaaggcatgaatctattctgaagtgcctcaattatttgattgaaaatcggcaggaagtgatggatattctcacgcaattttcatcctatcgggctgccaatgcagaaattgactccacgatccttacacttcaaggtgctctgcaagaagtgcaaacctatcagccaagctggcaacgatctatggctgtattcatgccttccaacgtcattttgtactcctatgcattatacctcttgattccgtccctgtacgttgaaaatatagactttcgcccttcttctcacgtaaacgagtacgtcaacatgctgcatgaaaagcttcaggctgtacacggattacccatctatattcgtaaggtcagtcaaagggtttttatggaaaattcggttatgccagctgatattgtggtgttcaccgggagctatgtcaatgcggagaagatcaaaaaacaaatccgaaaagaccaactctacatcttctatggacaagggatcaatcctttcgtcatcggacctgatgccgatctggaactggcggttaccgatgtaatccgaatgagactgttcaactcaggacaggactgcctgggacccgacatcattctggtacatcaagaggtgagtgagcatttcaaagatctgctgattcaacggcttgaccaacttgtgtttggagccaacgatgatccgaacgcaaactattcccctattttctataaagatgcgctgaactcggtttctgaatattttaatacgaacgataagtttattatttacggcggcggcattgatttccgtacgaaaaagatggagccgacggttgtatatagtgaattggatcagaatctggatattattgagtatttttcgccagtcttcaatgtagttagctaccaagacgacgagcaattgatcaaacgtatttcctccagttatttcagtgaacgtgcaatgggttgcagcttatacggatcggagcacctgacggacgttttacgaaagaagcacacattgaccattaatcagacgcttgaggatgtggatcaaggcaataagccttttggtggctacggaacgatgtcgaactatattttttacgatttcaagctcatttcgaagccgatcttgatttcagaaattgtcgcagagtatttgtcggaaaagaggagtttggtatga

SEQ ID NO: 63 - DNA, 1701 bp, Paenibacillus sp.
atgactgcgagcttctttccctgggatgccatcattggctacttaaaatcggctggggttcagcatctctttggattaccaagtgatgatctcaaaatcgtatcgtcactcccctcatccggtatggattttattctgtgcaaagatcaacgtaatgctgtgttcatggcagcaggttatgcgctcaccaccaaccagatgtccgtatgtgttgtggggaaaggccctgcgctcagtaatacactcacagggctgctggaagccaaaaacctgggcgctccgctcctgcttctggcgcttggtaccggaggagacaagctgggtacccgatcctttcaggaagccgatcaaatctcactcgtgaagccgctcgtaaaatgggcttatcgtgtcgagcatgtggatcggcttgtatgggcgatggagcgggctgcctttttggctatcaatggagcaccgggtcctgtctatatcgaactccctgaaaacctgatggaccagcctgtgccaacagggtttcagtttaccccaccagaaaagctgtcgtgctctccgtctcgtcgggagttggatgcatcgtggggaattattgagaacgcccgcaggccactcattctagtcggaggcggaatgaagtcaggtacaggtgatgtgattgagcggtttgctaaccaacatggagcggccttgttcgcaaccgcctcgggacggggtgtagttgatgaagaccacgaactcttctgtggtgttgcgggactctatacggatctcaatctaagacaaatctggaaggaatctgatgtagtgatcacactgggcagtcgattggaagaaacggctactttcgagtgggatattttgctacaggatacacctttgattcaagtcaatgtaaatctggaggattttgcacacgaatacaagggtatcaaggtgcttggtgatggatatgcagcggtggagcagtggcttgaacggcgcgggcaggagcctgatggagtgtggcttgaaacgattaggagttgtaaacaggaggctttttctcacagggctatttatttggaaaagctgaaaaagacccctggaatccacgttcctgagctgttggacatgattcagacggagttccctgaaaatctggtgcttcttcaagagaatggattgcaggatatgtggtcctacttttatccatattttcgctttgcccacggttctctttcgattgttcctagtgatcaaacgagtcttggattcggtgcggcggctgctctgggagcaagcgtggcaacacaacgtccagtagtagcactcgtaggtgacggtgcgttcaatctcttctgttcggattttatgacggcggtggagtatcaaataccgttaatctatctcgtactcaataatggtggctatggttggttgcagaatcagatgaattataaacatctcgcagggcagtccctcccgtttgtttctgaggcggccaagacaggaattcaaattccccagtatgagtttgtggaaagccttgcaattcggagtaagggagaggcacgggaacagcttcagcttgcttggcaaaagtatcaggagaacaagcttgtggtcgtcgaggtatatgctgatcttgcggatgtgcatgaaaaaatcagtcatgtgtatggcgactttcctctctacgagcaacaggcttccaagaattaa

SEQ ID NO: 64 - DNA, 1242 bp, Paenibacillus sp.
gtgagaagacgtgttgtagtgactggacagggagtcgtaacgccggtcgggcttaatcttacggatttctggaattcactgttggaaggaaaaagcggaatcggacctgtcactgctttcgatacagcggacattccgacaaagatcggtgcacaggttacgggctttgacccattgcaatatatgagcaagaaggaagctaataaagtcgcacagttcactcagtttgctttggcggctgcccggcaggcagtggagcaatcaaaattggtgattgatgagcggaatgcaagtcgcgtgggggttattgtcggctctggagcaggcggcctcgacgttatagaggagaactatgggaaattaacaaagcttggtcccaaaagagtttctccttattttgtttcctcgatgatgattaattcagcacctggtgaaatctccattgctattggagccaaggggccttcttttgctgtggttacggcttgtgccacaggaagtaatgccattggtgaggcattacgtacgatccagtacggcacggctgatgttgtggttgctggtggagcagaagccaattttagtcagcttgatctggcatcttttgcgaatatccatgctctctccagacgtaatgaagagccgcagaaggccagtcgtccgtttgatgtggatcgcgatggattcgtcatcggtggaggagcaggagtcttggtgctggaggagttggagtatgctttgaaccgcggagccaccattctggccgaagttgtgggttacggatgcacaactgatgcgtatcatatcacggctccagatccctctggctctggcccggcagaagcgatcaacatcgcactgcaggatggcggcttgacaccgcaggaaatcgactatgtcaatgcccacggtacgagtacgccatttaatgatcgtatggagattaacgctattcaaaaagtattcggggagcatgccccccatttggcgatatcgtctattaaatccatgactggccatttaatgggcggtgcaggggcagtcgaattaattgcgactgtgcagagcatgattcacagcaaagtaccaccgacactcaattgcgacaatcctgaagctcctgaattgaattttgtgccgcatgtatatcaggaacgcgaggtccgcgcggcgattagcaattcatttggtttcggagggcacaacgtctgccttgctgttcgcaagtgggaaggggaataa

SEQ ID NO: 65 - DNA, 441 bp, Paenibacillus sp.
ttgctgttcgcaagtgggaaggggaataagcctgtgagcgaagagctaatccatatcccggatgtgcttccgcaccggtacccatttcttctgttggatggcgtgaccgggtacgaaacgtcgcaatgggcgaagggatacaagcttgtcacctggagtgagtggttcattacagacactaatccatacatgcctcctatgttgattgtggagtcgcttgcacaattagcggcattcgctgcgattgataaaaaaggcatttcatacctgaccaacctcaatggaattcgattccatagcttggcgaagcctggggaccgaattgatctctattttgaagtgatcaagcgtcggcggggctatatactcggaagcggtcgggcttccgtaggcgatcgattggtggctgaggcggaagaaattgtttcactggatcaatga

SEQ ID NO: 66 - DNA, 23727 bp, Paenibacillus sp.
atgaatgacatgcaattatatgatttaacaaatgcgcagaagcgtatatggtataccgaattactctacccagatacgtcagtgtcacagctttccggtacagctaagatgaaggggcacatcaatattgctgcctttatgcagtccattaatttgattatcaaacagtatgatgcgttccgcattcgtattacctcagtggatggagtgcctcagcagtacgtcgttccttatgaagagagacagttggagtgcctggatcttagccactatgaaagtgtatctgaggtggaagccttacttgagcaacacaaaagcaaacccttgcccctgctggattctgagctcttccagtttttaattgtgaagattagcgaggaagagtattggattaatatcaagatgcaccatattatttctgacggaatatcaatggtggtctatggcaatcagctgacagcattttacatggagttaattcaaggaaatgaaccgaagctgggcgacgattgctcgtatattcaatatattgctgatgagaatgcatacgaactttctgacagataccaaaaagataaggcttactggctggataaattttctgatttgcctgagcttacgggttggaagtcatataatccgttatctttaagcacccacgccgttcgggagcattttaccgtaccagaagtgctatatcacgagctgcaagcattttgccaacagaacaggatttctttgttccagttcttcatgggtgcgatgtatatctacatacacaaaatgacgaatcagccggatgtggtgattggcacttcgttcgctaaccgggggaacaaaaaagagaagcaaaagataggtatgttcgtcagcaccgctgctgccagaacatacgtcaaaaaggatatggatgtgttgagctttctgcaggatgtagccagagatcagatgtcagtcctgcggcatcagaagtatccatacaatcagttaattcaggatcttagagaaatgcatggtaacaaggatattcagcggctttttggcgtttcaatggaatatcgtcttatcaattgggttgatttggatgatgtgcgcattttgacggattatgatttctgcggggacgaagtgaacgatttcgtgcttcatatcgtggagatcctggatgaaggcgaactggtactggatgtcgattatcggacggagctgtttgaacgcagtgaagttaaggacatggtttcccagttgcttacgatcgccgagcagatcattcattcacctcagctttctatcgcagaggtaaacttattgggtgaaccagaagagcaatccattttggctctttcggaaggcgctgcagtcgactatccacgtgagaagaccatccatggcttattcgaggaacaagccgagcgcacgccagatcacgtagccgttcagatggacgagcagagcattacatacctagctctaaacgagcaggctaaccagcttgcgagatatttgcgctccgagggagtagttgcagatacgctcgtagggattatggctgaccgttccttggagatggtcattgggatgttggccattttgaaagcaggtggtgcctatgtaccgattgatcccgattatcccgaagaacgtatccattatatgttggaggattcaggtgtccgtctgttgctcactcaaagtcatctatgggagagcaccgcttttgacggaaagcttgtgagtttggacgaggctgcaacgtatacaggagatgcttccaatctggagagtatttcgggaccaagccatctggcctatgttatctacacgtcgggtacgaccggcaagccgaagggcacgctgattgagcataaaaatgtagttcgactgctctttaacgataaaaatttatttgatttcagctctcaggatacgtggacgctgttccattcgttctgcttcgatttctccgtttgggagatgtacggagcgcttctttacggagggaaattggtgattgttccatctctcacagccaagagcccagcagctttcctggagttgttaaaagacaaccaagtcaccattttaaatcagacgccgacgtatttttatcaggtgctacaggaagagttagcgcactcttcgacagagcttagtcttagaaaaatcatttttggtggagaggccttaagcccatctcttttgagaaactggcgagtcaagtatcctgatgtgcagctgattaatatgtacggaattacggaaacaacggtccatgtcacctacaaggaaatcacggaacatgagattgaagcggggaaaagcaatattggcagaacgatccccacacttagcgcttatattctcgatgagcaaagacgcctacagcctgttggggttccgggagagctatacattgcaggggacggtcttgcccgtgggtatttgaatcggccggatttgacgtctgagaaattcgttgagcatccgtatcgggcgggagagcggctgtaccgaactggggatcttgctcgttggttgcctgatggaaatattgagtatttggggcggatcgaccatcaggtcaaaattcgcggctaccgaattgagcttggcgaggtagaagcccaaattctcaaggctccgaatgtacgagaaacgattgtcctcgcacgggatgacgaacagggccaaaaattgctgtgcgcctactatgtagcctctagtgatctttcgccgggggaattgcggtctcagctggcagcggaactccccgcttacatgattccttcttattttgtccggctggagcaaatgccgcttacgccaaatggcaaactggatcgccgtgcgttgccggctcctgaaagcagcgtacaatccggcgaggcttatttggctccgagaactgctgtggaagctcagatggtactcatctggcaagatatcttgggagttgcccgcgtcggtgtcagagataatttctttgaaattggtggtcactctttgcgggcaacagtgctcgtttcacggattcacaaagaattgggatgtagcatttcgctgcgtgaggtgtttcagtcacctacggtcgaatccttggcgcaacttgtgaaaaaacacattccgaccatgtacgaaactatcccacaggcagcggaaagcgaagcttacccagtatcctcagcgcaaaagcgcttatacgtgctgagacagatggatgggggagagcttagctacaatatgccaggggtcttcacagtggatggaccgttggatcgcacgcggctggagtccgcgttccaggcactgatccagcgtcatgaatccctgagaaccggcttttatatgcaggatggagagcttgttcagcgtgtgcataggaatgtgccgttcgcgttgaactacacagaggcttcggtggaggagacggatacgctcattcacagctttattcgtgcctttgatctgagccaggctccattactgcgtgttagcttggtgaagctccaggaggagcgtcatctgttgctgtttgatatgcatcacatcatttcagatggggtttctattcaaatattgatacaggaacttactcatttgtatcaaggagaacagctaccagaactgcacatccaatacaaggattatgctgtatggcaacgggaacagtcagagaatcaatggcaagatcttgagaaatattggctgcaatcctttgaaggagagttgccggtattggatttgcctacagacttccaacgaccttcagttcggagcttcgagggtagccgaattgattttacattggatgagtctggaaataaggcgatacaagagctagcatcccgtacaggtactacactgtatatggtattgctggccgcttattcggtactactgcacaaatatacaggacaggaggacatcgtcgtaggttctccagtagccggaagaccgcaggctgagcttgagggcatcatcggaatgtttgtcaacacactggccttgcgcagctacccggcaggagataaaacctttgaggattatcttctggaaatcaaggaaacggcgctcaaggcgtttgagcatcaggattacccttttgaaaaattggtcgaaaagctgggcgtaggacgtgatgtcagccgcaatccgctctttgacaccttattggtattacaaaataccgagcaggaagagcaggatatggacggagtgcactttactccttacttgatggacaccgtcacagccaaatttgacctgtctctcaatgtagaggagaagggttcaaaattagcctttggcctcgagtatagtacggctttatatcggcgtgaaagtgtagagcgacttgcaacgcacttgctccgggttctgcatgcagtctcggccaatcctcagttgcaactggccgagattgaaatgatcacaccggaggagaaagtacagatcgttgaagtatttaacgcgacatcggctccttatccaagggacaagaccattcatgagctgttcgcagaacaagtcaagcgtacaccagagcagacggcgcttgtattcggcgatgtccagctaacgtatcttgaattggaagacaaagcgagccgactggcccaaacactgcgtcgtttgggaacgttgagggagcagcctgtggccgtgatgggcggacgaagcatcgagatggtcattggtatgctcgcggtgcttcaagcaggtggagcctatgtgccgattgatcctgattacccggaagatcgggttcgttatatgcttaatgattccgacgccaagctattattggtgcaaaagggcgagcttataagtgtagactacggtataccgattgtcgatcttagcagtgaagaggcttatgctgctgagcctgcccagccggagacggctcagggatcgcaggggcttgcttatgtcatctatacatcgggtacgacgggtagaccgaagggcgttatggttgaacaccggaacgtggtccgtctggtcaaagagaccaactatgtggagctgaatgaatccacacgaattttgcagacaggagccgtggccttcgatgcttctacctttgagatatggggagcgttgcttaacggtgggcaactctattttgtagagaatgacgacattctgattgctgataggctcaaagcggctattgccaagtacgggattacaacattgtggcttacttcaccccttttcaatcagctttccctacaggatgagtacctgttcagagggctaaaagcattgttagtcggcggtgacgtactgtccctatctcatatgaatcgtgtaatcgaggctaatcctgatcttgtccctatcaattgctatggtccgacagagaatacgaccttctccaccacctacaagattccgggtcgtgccgaagggggtgtgccgattggtcgcccaattagtaattcgaccgcttatgtggtcaatggatcgctgcaattacagcctattggtgcttggggtgaactcattgtcggcggtgaaggtgtagcgcgcggatatctcaatcgtcctgatctcacagcagagaaatttgttcctagtcctgttaaggacggagaaccctgctaccgaacgggggatttggtacgctggcttccagatgggaatttggagtttaaaggaagaattgatgagcaggtcaaaattcgtggttaccgcatcgaactccctgaaatcgaggcccaactggccaaggtggagtcagtaatcgacgccgtagtggtcgttcgagcggatgagcttggagagaagcagctttgcgcttattatgtggcggatcgtacgctcacggcaggcgaagtacgtctttccctatcgcaggtacttccgggctatatgattccatcctactttatccagatggaccgtatgccattaacgtcaaacggaaaagtggaccgcaggtctctgccggctcctcaagtaggcgcgcatacaggacggaagtatacagctcctcgtacaccggctgaagaagctttggcatctgtctggcaaggggtactgggtgccgaacaggtgggtatccatgacaatttctttgaattgggcggagactccatcaaggccattcaggtgtcgtcacggttactgcaggccggctatcggttagagatgaagcagctgttcaaatcgccaaccattgccgagctgggcgcggaaatacaaacggctgtgcatatggctgaacagggagttgtgcgtggagcgactcgcttgactccagtccaacagtggttctttggacggaagcaggcagagcctcatcacttcaatcaagcggttatgctgtatcgtgaacaagggtttgaggaaaaggccttgcatcaggtgctaagaaaactcgctgagcatcatgacgcccttcgcatggttttccgtcagacagagcatggctacgaagcttggaatcgtgatcttgaagaaggagagctttatagcctgttcaccgctgatttacggaatgaatccgatccggctgcagccattacatcgctgtcggatgacattcagcgcagtatcaatctggcagaaggtccactgctgaagttagggctcttccattgtcaggatggagaccaccttctgatcgtgatccaccatttggtggtggacggagtatcctggcggattttgttcgaggatattgcagccggctatgagcaggtgattcaaggacaagcgctgacattcccgcagaagacggattccttccgtgactggggagacgcccttgctcgttattcggaaggtcctgaaatggagactcatcgggcgtattggagagagctggaggatcagccactcgaacagttgccgaaggatgagactgtggaaagccttcttttacaagatagcaaagtagtaacagcacaatggactatagaagaaaccgaccaattgttgagaaaagcccatcgtgcttatcaaacagagacgaatgatctgctattgactgctctgggcatggcggtatccaaatggtctggcatcggaaaggttgctgtaaatctggaaggacacggtcgtgagccgattataccgaatatcgacatcacccgtactgtaggctggtttacaagtcaatatccggtgattttagacttgggggatgacccagaagtggcctccttgatcaagtctgtgaaagaggggctgcgccagattccgaacaaaggtattggctacgggttgcttaaaacgatggcaagtcagttggatgaagacagcttcagcttgcagcctgagatttcttttaactatttggggcaatttgatcaggatttgcaaggcagctcgttgcagatttctccttatccgaccggaagcgcccaaagcttgttggaggaaccggcctatacgctagatatcaatggcatggtgacggacggagccctgacgcttacgattacttataacggaaaacagtataagttatctacgatagaacagctcgctggatatattgaagaaagcctgcgggagcttctccagcattgcctaacccaagaaaaaaccgtattgacaccaagcgacgtgcttgcgaagggtctaagcattgccgatctggaggagctttctaagcagaccagccacataggcgatattgagaatgtatatagtctgacgccgatgcagaagggcatgctgttccatgatatgtttgagccgcatacaggtgcttattttgaacaggctgcctttgactttaagggtagctttgatccgaccgccttcggacacagtctggatgcagtggtggagcgtcatgccatcctgcgcacgaacttttacagcggctggggcagcgagcccttgcaggttgtatttcggcacagaggcgctaaattggtgtacgaagacctgcgggagatgaatgcatcgcagcgcgaagcttacctgaagacatttggtgctaaggacaaagcactgggcttcaacctggctgaagacgagcttctccgtgtatcaattttacaaacagatgaagagagcttccgcctcttatggagctttcaccacatcgtcatggatggctggtgtgttccgttaattacgaaggaggtatttgaacactattttgccctcctggaaggaagagagccgcagctggcagaggttcatccgtacagtcgatatatcgaatggctggaacagcaggatgaagcagttgcgtccaactattggagccgatatctggccggttacgagcagcagacgcttttacctcaagtcggtggagcaagtaagggagaaggctatgtagcagaaaagctgaattatcctctcagcagggaattgactgagcgccttgaaaaggtggccagggatgctcatgtcacgatgaatatattgctgcagtccctctggggcattgcgcttcaacgctataacggtagccgggatgtcgtgtacggaagcgtagtatcaggcagaccagcagaaattccgggcattgaccggatgatcggtttgttcatcaatacgattcccgttcgtgtgaagacagaggagaatctccccttcaccgttctgatgaagcagcagcaggaacaatatatggcttctcatatgtatgacacctacccgttgtttgagattcaggctcagacggatcagaagcaggatctaatctcccatattatggtgtttgagaactatcctgtggaggaggaggtagagcgtctgggtggtggcgaggctgcctttgagattgaggaagcggagcttcttgagcaaacgaattatgattttaatttaattgtcctccctggcgaagagatgagattgctgttccagtacaatgcacttgtttatgaccaagtgacaattgggcaaatcaagggccatctggttcaccttatggaacaaattgtagagaaccctgctatttccgtggatgctctagaattagtcacgccgcaggagagagaacagattctgaacgtatggggaaatacgaaaggcatttacgagcactgtaacacgttccacgggttgttggagcaacaggtgggacgaacgccggatgcgactgccatttggttcgaggacgagagtctgacctatgccgagctcaatgcaaaagccaatggactggcaagaaggctccgtactcagggaatcaagacgggagatctggtgggactgattgctgaacggtcgctcgaaatgatcgttgggatctacggcattatgaaagccgggggtgcctatgttccaatcgatccagagtatccgaaagaacgaatcagttacatgcttgaagattccggggcgaagctgatccttacacaggcccatttcttggggcatctcggatggacggaaaatgttttgctgctggatgaatcatcgacctatgatgccgatacctcgaatttggaggatactgctggcccggatgatctggcttacgtgatctacacttcaggtacgacggggcagcctaagggcgtattagtcgagcatcggggactaccaaatctttcggacgtatatggggcacacttcgaagttacaccgcaggatcggatcgttcagtttgcaagcctgtcgtttgatgcatcggtttcggaaattttaacggcgctgagccacgggggtgttctgtgcatcccttctacacaagatattttagatcatgccctgttcgagcagttcatgaacgataaggggattacggtagcgactttgccacctgcttacgctatccaccttgatccagagcgtttgccaacactgcggtgcctgctaaccgctggatcggccgcatcggtcgagttgatcgaagagtggaggaagcatgtacgttactctaatggctatggcccaacggaggactccgtatgcaccaccatctggtctgtcccggacagtgaggaagcaacggatattgtatctattggacgacctattgctaaccatagtgtgtacatcttggatgaccattttagattgcaacctgtcggtgtagctggagagctatgcatttcgagtatcggattagcacgggggtatcataaccggcctgagttaatggatgagaagttcgtggacaatccgtttgctccaggagagcgtatgtatcggacgggtgacctggttcgctggttaccgaatggaaccatcgagtacttaggccgaatagatcaccaagtcaaaatccgcggctaccgtatcgagctaggcgaggtagaagcacaaatgctcagagtgccatccgttcaggaagtcgtagccatggctgtagagggcgatgacggctacaaagatctagtcgcttatttcgtagctgctcagaaacttgaggtatccgagcttcgggccgtcctgtcggagatgttacctggatatatgatcccttcccgcttcatacaactgaaggatatgcctctgacgtcgaacggaaaaatcgatcgaaaagcgctgcagggcgagcgtggatgggcagcggcttcatcggaggctccaaggacacctgtggaaatccaattagccgaaatctggcaagaggtgctgggtgtagagagcgcgggattgaaggataatttcttccattttggaggtcattcactgcgtgcagccctgctagtctcacgaattcgcaaggaaatgaatcgcgagattagtctgagagcagtgttcgagtctcctaccattgaaggattggctcgtgccattgagggctatacaccgctgaatttcgaagaaattcctacagcaggagcgagagagcattacccattgtcctcggctcaaaaacgactgtttattctaagtcagctggaaggtggagagctaagctacaatatgccgggtatccttacggttgagggagctttggatcgggaacggctagagcaggcattccgtcgtctgattcatcgtcatggttcgctgcgtactcgttttgtgactgtgaacggtgaacctgtacagcagctcctgacggatgttccgtttactgtggaatatgcggagttgagcgaggaagaggcaggagctacccttcagcagtttgtccgtcctttcgatctaggtgtagctccattgctgagggtcggtctcattcgaattgcacatgagcgccatttactattgtttgacatgcatcatattgtctcagatggggtttctatgaatattctcatagaagagtttctccgcttctaccaagaggaggacgtatttcctgagctacagatccagtacacagactatgctgtatggcagcaagagcagctcggaagcgagcgtcttaaggcccaggaagcttactggctggatgctttccgcggaagcttgccagtgctggatttgccaggagatgaagttcgtcctgcggtgcgaagctttgcgggcgatcgaatcgacttccaaattgattcttctctgagtgcttcacttcaggagctggctacccgaacgggttccactctgttcatggtactgctggcagcctatacagcgctcttgcacaagtacacaggtcaggaagatgtcattgtcggttcacctgtggcaggaagatcccatgcgacactcgaaggcctcatcggtatgttcgtcggcacagtggcacttcgtacttatccagaaggagaaaagcctttcgaggcttatctgcaggaagtgaaggaaacagcgctgcgggcctatgaaaaccaggattacccgttcgaggagctggtagaaaagctggagcttcagcgtgatttgagccgtaacccgctatttgataccatgtttgtcctgcaaaatatcgagcagggagaacaagaaatagaaggattgcgcttcactccttacgataatgtacatccggctgccaagttcgatctcacgctgaccgtgagtgaagtagacggggcattgaactgcacgcttgagtacgcgactgcgatctacaagcaagagactgctgagcggatggcaggccactttgtacagcttattcgggaagccgtctccaatccgggaatgccgttgtcatcccttgatatcgtgacacctcaggaaaaatcaaggctgatgaaagcgccggacgaagccaaggcagattatcctcgtgacaagacgatccatgcgctgttcgaggagcaggccggacgtactccgaatgcagtggcagtcgtatgtgaagatgcaaccctgtcctacagcgagctgaacgagcgggccaatggacttgcccgaacgctgagggaacgtggtttgcaaccagacggtttggctggaattatggcggatcgttcccttgaaatggtggtcggaattttagccatcttgaaggcaggcggggcctatgtccctgtagaccctgaatatccagaggaccgcattcgctttatgcttgaggattcgggagccaagctactgctgacacaagcgcatctggagcaacgtgtctccttcgccggggacatcgtgagtctggataaaacggcttcctacaaggaggatgtctcaaacctgcagcctgcagccggaccagagcatcttgcctacgtcatctacacatcgggtacgacaggcaagccaaagggaacgctgatcgagcataaaaatgtagttcgcttgctctttaatgataaaaatatgtttgactttggtcctcaggatacgtggacactgttccattcattctgttttgacttctctgtatgggaaatgtacggagcattgctgaacggaggacggttggtcatcgttccatcgcttaccgcgaagagtccggatcgtttcttgcaattgcttaaggatcagaaggtcaccgttttgaaccagacaccgacgtacttctatcagttgctacaggaagagctcggtcatcaggcggcagaactgagcctccgtatgattatcttcggtggagaggcattagctccggccctgctcaaggactggagaacgaagtatccgcaagtgcaactcatcaacatgtacggcattaccgaaacgaccgtacatgtaacctacaaggaaattacagagttggaaattgaacagggccgcagcaatatcggcaccacgattccgacgctgcgagcgtacattttggatgaacaacgccgtccacagccgattggcattccgggtgaactctatgtggcgggcgtaggcctggcgcgaggctatctgaaccgaccggaattgacggaagagaagtttgtcgctcatccgtttgaagcgggcgagcgcatgtaccgctcgggtgacttggcacgctggttgccggatggcagcatggagtatttgggacggattgaccatcaggtaaaaatccgtggttaccgtatcgagctgggcgaagtggaagcgaagttgctccatgctccgtctgtaagggaggccgttgtgctcgcccgggaggatggaagtggacaaaaagtgcttgtcggctatttcactgccgatcagatgctgacggtaggcgagttgaggaaagccttggctgctgaactgcctgcttatatgattccatcttactttatgcaattggaacagatgcctttgacgccaaatggcaagctggatcgcaaagcgcttccggctccggaggccaatgtgcagactggagcggtttatgaaccgccaaggacgaaggctgaggaagctttagtttccgtatggcaaggtgtgctgggagcgcagcaggtaggcatccatgaccatttcttcgatctgggtggtgactccatcaaggcgattcaagtgtcctcgagattgttccaagctggatataaattagagatgaaggatctctttaaatatccgacaattgccgagctaagcccgtatcttcaggcagccggacgtacagcggaacagggcgaaattaaaggtgcagcagagttaatgccaattcagcgttggttctttgaacgccatacagcggagccgcaccattataatcatgccgtcatgctctatcggaaagacggctttgatgaggctgcactccggttgacaatggatcaaatcgcgatccatcatgacgcgctgcgcatggttttccggcctacagaagctggatacgcagcttggaatcggggaatggacgaaggcgagctctacacattggacattgccgatgtgcggcaggcggaagaccagacagctgcggttcaggcccaagccgatgccattcaggcaagctttgacttgaaaggtggcccactgttcaagctaggcctgttccattgtgacgatggcgatcatttgttgattgtcattcatcacctcttggttgacggcgtatcctggcgcatcctgtttgaggacattgcagcggggtacgagcaggcattgaatggacaagcaatcgtccttccacaaaagaccgattcgtatcttgtatggtctgagcaagcgacgaagtatgcagcaggggctgctctggacaaggagcgtgcatactggcagctgatcgaggaggcgattttggccccactgccgaaggatgaggatcataagccgggcaccattcgggatactgaatcggttacggtaacgtggtctgcgcaggaaacagacctgctgttgcgacaagcgaaccgggcgtatcatacggagacgaatgacttgctcttgactgctctgggggcagccattcagcgctggacaggcatggagcagattttggtcaatcttgaagggcatggacgggaaatgatcgtaccagacctggatattacccgtaccgtgggttggttcacaacccagtatccggttctgctgaacctgcaaggcagacaggaagtatctgcgcgaatcaagcgcatcaaggagaatttgcgagaggttccgcacaaaggaatcggctacggtcttctgaagtatatggcaccggagaaaagtgtcggattcggcgtagagccggagatttccttcaattatcttgggcagtttgatcaggatttggagggtaatgctcttagcctatccacacattcagttggtaaggcgctcagtgatctcacacctcagcaatatgctctggatgtgaatggcatgattgccgaaggccagctatcactgacgattacgtacagcagcaggcagtatcgtaaggagacggtgagccattttgccgaattattacagtcaagccttagtgaggtcatccgtcattgtgtggctcaggagcgttcacagcttacacctagtgatgttctgttccaaggattaacattggagcagcttgatcgactcacggcgcagacggctcacatcggagagattgaggatgtgtacaagctgacgccaatgcagaagggaatgctgtttcacagcctgctggagccggactcctcttcatacttcgaacaggcaacgtttgagctgcgtggcagcttcgatgtagataccttcttcgagagcttccaggctttggtgcaacgacatgccatactacgtaccggattttacaacaacattgctgatgtaccgctgcaagttgtctttaagcaacggttaatccctctgcactacgtagatttgcgcgacgcatctatgcaggaacaagaagcccgaatcaaggcttatattgctgaagatatggttaaggggtttagcttgtcagaagatccgctgatgcgagtgaccgtcttgcagaaagatcaaagctgtcttgtattgtggagcttccaccatattgtcatggatggctggtgtatcccgatcattacacaggagctgttcgattattattctgccaagaaacagcaaatacagcctgtcctgccgccagctcagccctatagccgttatatcgagtggcttgatgcacaggatgatgaagaagcttcaacgtattggagccaatatctcgaagattatgacgggaatactgtattgccggaaggtaaaacgaaatctcaagccaaagaggcgggctatgttctgaatgagcatgttctccatctgggcgcatccttgacaggtaaaatggatgttgtcgcgaagcgcaatcacgtgaccgtcaacacactcatgcagacagcttggggactgattcttcaacgttataatgccagctcggatgtcgttttcggcggcgttgtgtcgggtagacccgctgagattgcagggatcgaaaatatggtgggcctgttcattaatacagtacctatccgtgtacagtcatccaaagacgaagcctttgtcgaggtgatgaaacgtacacaggcacagtcattggctggtcgtgcctacgacacctatccactgtatgaaattcaggggaaaacaacccaaaagcaggacttgatttctcatattatgatctttgaaaattacccgctcgacgagcaggtggagcaatcgggtaatcaaacggaggacaatctcgaagtcgccaacttcaccatgtttgaacaaaccaattatgactttaacctggttgtgattccaggcgaagacatcaaggtctgcattcgctataatgcttcggtttacgagcaagaaagcattgcacgaatcggaggacacttgttgcagatgctcgatcaggtggctgctcgtccgcaggcgacgatacaggaactggagattgtaacatctgaggaacggatgaacctgcttgactggggcggcaaggcccatacctatccaagtgatcagggtctgcataccttgtttgaagaacaggttgtccgtacgccggataagattgcggctgtaaacggggacactcagatcacgtatcgagagctgaacgagcaggcgaacagactagcttccaccttgatagaccaaggactacggagtgaacaagtggtaggtctgttggcagatcggtctgtagagctgcttgtcgctatcatgggtgtgctcaaagcgggtggagcctatgtacctattgatcctgaatatccgcaggagcggattcagtatattctgaaggattctggcgctgaaattctgctcacacaaagccacctgacggagttggcctcttttgaggggacggttatggaattggattcccggcacatctacggaaccgaggtggataatcccaatattcctgttggaggaaacgatctggtgtatttaatctatacctcgggtacaaccggaaatccgaagggaaccatgattaaccacaaagggatcgtgaactacatctggtgggccaataaggtctattgtgccggggagccaacggatttcccgctgtactcatccatttcctttgacttgacgatgacatcaatgtttactccgttaataaacggaggaatagtacggatttatgatggtatagataaagcggaggttgttcagcatattttgcgcgaaaatgcggtggatattctcaagctgacgccgacacatctcagtctgattaaagatatggccattccagcggaaagccgcattcagcagctcattgtgggtggagagaatttgaccacacatttgtcgaaaaccatcacagatctctttggcggcaacatcaaaatctacaatgaatatggaccaacagaaaccgtcgtcggctgcatgattcacctgtataatcctgcgaaggatacgcgtgaatctgtaccgattggactgccagcggacaatatatacattcatatcctggatgaacagcttcgtctcgtaccattaggcgtggagggcgaaatgtacatcgccggggacggggtagcccgtggatatctgaaccgtcctgagcttaccgcagataaattcattagaaatccattcgcttcggaaggaaatatgtatcgcaccggggatttggctcgtcgccttcctaatggagacattgagtacattggacgcattgaccatcaagttaaaatacggggctatcgtattgagcttggtgagattgaggccaagctgctggacattccacttgtcgaggaagctctcattgttgcgtgggcagatgctcatgggcagaaatcgctgtgtgcttacttcgtagctgatcgcgaaatgtctgtcagcgagctgagaaacgaactgtctgccggactgcctgcatatatgattccgtcttacttcgtccaattggacgtgatgcctctgacaccgaatggcaagctggatcgcaaggcactgcctgaaccgaactcaggtataaaggcgggagcagactttaccgctccgcagacggatgtggagaacattttggcttcaatctggcaaggtgtactcggcgtgccgcttgtcggcattcatgataatttctttgagcttggaggtgactcgatcaaatccattcaagtatcctcaaggcttctccaagcaggctataagcttgaaatgaaggatttgttcggttatccgacaattgcagagttggcgcagcgcgttagtgtagtcagccgaattgcggaccaaagcgaggtacacggagcggtaagactgggacctgcccagcacagattcttcgatgaacagtcgatggatctgcatcactttaatcagtcggtcatgttgtaccgacgggatggcttcaataccgatgcgctcgccgaggttgttcggaaaattgcagagcatcatgatgctttacgactgatgttccgccaaggagagcagggattggaggcctggaaccggagcgtgggtgagggagaactctatagcctccaaatccacgacctgcgggatgaaacagacccggcttcagcaatagaagcaggtgcggaagccattcagcgcagcatctctctggaggatggacctctctttagactgggtctgttccgctgtgcggaaggagaacatttgttgatcgttattcatcatctggctgtggatggcgtatcctggcgtattctctttgaggacctgcaggaaggctacgagcaggcagcacgtggagaagcggtcaagcttccacagaagacggattcgtaccgtgcatgggttgagggaatcacacaatttgcaaacagcccgacggctgaacaagaactcagctattgggcagaagtagagggagatggctttgtccctcttcccaaagataaggtagacggcgctcttctcatcaaagacagtgaggctgtcacggtgagatggtcaccagaagagacagagcagttcctgaaagaagcgaaccgcacttacaatacggaggttaacgatctgctcctgacggctctaggtatggctgttcacgagtggacgggaatcgaacgtgtaggcatccttctggagggacatgggcgggagcctgttgtaccggaactggatattactcgcacaataggctggtttacaagtcaataccctgtcgcccttgtgatgggaggcgaattggagatcggcgccagaattaagcacgtcaaggaaggcttgcgtcgtatcccgaacaaaggtgtcggatatggtattttgaaatatttaagcgacggttccgatgtctcctccttctcggctgaaccggagattaccttcaactacttgggacagttcgaccaggatcttgcaggagggacgatggaagtatcgccttactcagtaggacctgaggtcagtgagcagatggtgcagcatcaggcattgaacattaatggactgattgccgaaggacagcttcaactttcggtcagctataaccgtcatcagttccacggggagtccgtggctaagtttgttggcattctgaagaaccgtctcagcgaagtcattggacattgcgtaagtaaggaaagaacagaacttacaccaagcgatgtactcctcaaagatatcagcttggaaaaaatcgaggagttagaagagcagacacgacatatcggtagtattgaaaatatgtataaactgacaccgatgcaaaaaggaatgttgttccacagcttgctggagcctcattcggaagtctactttgagcaggccaagtttgaaattcagggagcattctatcctgaggattttaaacgcagcttaaaatatctgatgaaacggcatgccatattgagaaccaatttccatgccgggtggggcgatttccctattcagattgtgttcaaagaaagagcgtgtgacttcgtatacgaggatctgcacgagctggaagccgatgaactccaagcgcgtcttgcagcttatactgctcaggacaaagcaagaggctttaatcttgctgaagaagcgttgctgcgtgttgctattctacgtacagcagaagaggcttaccatttgctgtggagctctcatcacatcattttggatggctggtgtatgccgcttgtgctccaggaagtgtttgagacgtatggggttctgcgtgagcaaagggaacctgagcttcctgcagctgtatcgtacagtcagtatattcaatggttggagaagcaaggcgaggaagaggcatcctcttactggagagggtacctggaaggttacgagcagcagacgaagctgccgcaggccatcacacagccatcggcaaaagcggaagcctacgtgtcggagaagctggtattcacgttggatgcggaactgaccgatcgcctggaacaggtggccaagcagcatcaggtgacgatgaatacgctgatgcaagcagcctggggaattgtgttgcagcgctacaatagaagccaggatatcgtcttcggaagtgtggtatcgggaagacctgccgagattccgggtatcgaaagcatgatcggtctcttcattaatacagttccggttcgggttcaggcagagggaagcgattcgttctcccatgtgatgaaaagacagcaggaattatatttggcaggacatgcttatgattcctatccgctctatgagattcaagcacagagcgaacaaaagcaagatttgatttctcatattatggtgttcgaaaattacccggtagaagagcatctggaagagaaaattgccagtgaagaggctgaatacaaaattacggatgttcagatgtttgaacagacgaattatgattttaacctcattatgctgccgggtcgtaatctggagttcttgtaccgttacaatgcccgcgtctatgatcgggagagtgtggaacgaattcaaggacacttgacgagaattctgacaagcgttgctgttcaacctgctatccgtattgatgagctggagctgatcacgccagaagagaaatcgcagattatagaggtgtggggcgatacagcagctccttatccgcgtgagcagaccttacacggtatatttgaggaaaaagcggcactcacaccggatcgtacagcacttatttacggtgaaacggagcttacctatggagaacttcatcagcaggcgaaccgccttgcacgtacgctgcgtgcccaaggggtcagaccggaccaaccagtcggcatcatggtcgagcgctcgcttgagatgatcattggcattcatgccattctcaaagctggcggggcctatgtgccgattgatccggagttcccggaagatcgtattcgtcacatgctggaggattcgggagcgaagcttctgctgacgaagaaccatctcaaagatcgttttcagttcactggcacgatcctggcacttgatgatccgcagacgtatcatgcggatagctcgaatctggagccaattgcggggccggagcatctggcgtatatcatttacacgtcaggttcaaccggcaagccgaagggcgtaatgattgaacatcgcgctgccgtccatacgctgagtcagttggaagctgaatatccgatgttggcaggcgaccgtttcctgctcaaaacgacattcacctttgacttctccgtgccagagctgttctgctggttctttggacaggggactctcgtgatcctgccgcaaggcgtggacaaagacccgatggcactgctagaggccgtggatacgaaccgtatcacgcatctcaatttggtgccgtcgatgctcagtgtgctcgttcaatacttgaaagaaagcggcaaccaaggattccttactctgaaatacctgtttgcctgcggcgagacactgcctgccaaacttgtggaagagtattatagagtatctccttatgcagtactggaaaatatctatggtcctacggaagcagccgtatatgcgactcggtatacaacgagccttgagactgcggctctaacgcatgtgccaatcggcaaaccgtacgctaacgtccaagtatggatgatggacagcgcttctcaggtatcacctgtgggggtaccgggagaactctgcattgcgggcgaaggggtagcgcgggggtatttcaaccagccggacctgacggcagagaagttcattcctcacccgtacaaaccgggagcacgaatttaccgaacgggcgatttggcccgatggctgccagacgggaatattgagtatttggggcggatcgatcaccaggtgaaaatccgcggttaccgcattgagctgggagaagtggaagcacaaattttgaaagtgccatctgtgcaggaagcggttgttcttgcactggctgattctactggaagtactcagctttgtgcatactttgtggccgaagaggggcttgcagcgggcgtactacgtgaggcactggccagcgagctgccaagctacatgattccgactgctttcgtacagttggcacaaatgccgctgaatccgaatggcaaattggatcgcaaagcgctaccggcaccggaaacacttctgcggagcacagcggagtatatcgcgccgcgtacgcagacagaagtagagctcgctcagatttggtccgaggtgctcggcgtacaggaaatcgggatcagggatcatttcttcgaacttgggggccattccctgaaagtattgggcttgatccaaaggatctcgtccggtatgggcgtacagctcccactccaagtcgtgtttaatctgccgactgtggaagaaatggcgcatgaaatttccaagctgcaggcaacaactgctgctaatgaagaagaaatggaaattatccgcttcccagggaaaggaacgctcaaagtgttttgcttccctccacgggtaggccactctctggggtactatgagatggccaaggagctggatgggctttgcgaggtgtacgggatggaatttatcggcgaccgtttccagggtcaagatatgctggatcgatacatcgatgccatcgtggatattcaagcagagggcccatatgtattcctgggatactcacttgggggaaatctcgccttcgaggtagctaaagccatggaaatccgaggtcaccatgttggcgaccttattatggtagatgctatgagaaagatgtccaaggatgaatcgacaccagaggagcttgaagagattgtcgagacggtgctggacagcattagagagcagtacaaagtattcctcgcagatccagcggacagggagcgagtcatggataaaatgctggtttactctacctaccgcgatgagcttattaacgcaggtgaagttcatgcgaatatccatgctctgattgcagaagatgatagtattggtccggatacatcattagataaattgttatggcaacaggcgacacttggccaatacaaagaatacgaagtcatcggaacgcatgatgtgttgcttgattccggttttgtcggggaaaatgctaaagtactgagacagatacttggcaaggtcacagaggcctcatctaaaaacaagcccattttgtcctaa